(19) United States
(12) Patent Application Publication
(10) Pub. No.: US 2019/0298580 A1
(43) Pub. Date: Oct. 3, 2019

(54) LOW-ACUITY DRESSING WITH INTEGRAL PUMP

(71) Applicant: KCI Licensing, Inc., San Antonio, TX (US)

(72) Inventors: Colin John HALL, Poole (GB); Christopher Brian LOCKE, Bournemouth (GB); Richard Daniel John COULTHARD, Verwood (GB); James Killingworth SEDDON, Wimborne (GB); Timothy Mark ROBINSON, Shillingstone (GB); Matthew BISPHAM, Amesbury (GB)

(21) Appl. No.: 16/380,647

(22) Filed: Apr. 10, 2019

Related U.S. Application Data
(63) Continuation-in-part of application No. 15/571,238, filed on Nov. 1, 2017.
(60) Provisional application No. 62/159,110, filed on May 8, 2015.

(51) Int. Cl. A61F 13/02; A61M 1/00

(52) U.S. Cl. CPC A61F 13/0223; A61M 1/0035; A61M 1/009; A61F 2013/00174; A61F 13/0209; A61M 2207/00; A61F 13/0216

(57) ABSTRACT

Systems, assemblies, and methods for providing negative-pressure therapy to a tissue site are described. The system can include an absorbent and a sealing layer configured to cover the absorbent. The system can also include a blister fluidly coupled to the absorbent. The blister may have a collapsed position and an expanded position. A first check valve may be fluidly coupled to the absorbent and the blister and configured to prevent fluid flow from the blister into the absorbent if the blister is moved from the expanded position to the collapsed position. A second check valve may be fluidly coupled to the blister and the ambient environment and configured to prevent fluid flow from the ambient environment into the blister if the blister is moved from the collapsed position to the expanded position.

### RELATED APPLICATIONS

This application is a Continuation-in-Part of U.S. patent application of Ser. No. 15/571,238, entitled “Low-Acuity Dressing with Integral Pump,” filed Nov. 1, 2017, which is a National Stage Entry of PCT/US2016/031397, entitled “Low-Acuity Dressing with Integral Pump,” filed May 8, 2016, which claims the benefit, under 35 USC 119(e), of the filing of U.S. Provisional Patent Application No. 62/159,110, entitled “Low-Acuity Dressing with Integral Pump,” filed May 8, 2015, which is incorporated herein by reference for all purposes.

### TECHNICAL FIELD

The invention set forth in the appended claims relates generally to tissue treatment systems and more particularly, but without limitation, to a dressing having an integral pump for low-acuity tissue sites.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” and “vacuum-assisted closure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, the cost and complexity of negative-pressure therapy can be a limiting factor in its application, and the development and operation of negative-pressure systems, components, and processes continues to present significant challenges to manufacturers, healthcare providers, and patients.

### BRIEF SUMMARY

New and useful systems, apparatuses, and methods for providing negative-pressure therapy are set forth in the appended claims. Illustrative embodiments are also provided to enable a person skilled in the art to make and use the claimed subject matter. For example, a system for providing negative-pressure therapy to a tissue site is described. The system can include an absorbent and a sealing layer configured to cover the absorbent. The system can also include a blister fluidly coupled to the absorbent. The blister may have a collapsed position and an expanded position. A first check valve may be fluidly coupled to the absorbent and the blister and configured to prevent fluid flow from the blister into the absorbent if the blister is moved from the expanded position to the collapsed position. A second check valve may be fluidly coupled to the blister and the ambient environment and configured to prevent fluid flow from the ambient environment into the blister if the blister is moved from the collapsed position to the expanded position.

Alternatively, other example embodiments describe a dressing assembly for providing negative-pressure therapy to a tissue site. The dressing assembly can include a pouch and a cover configured to cover the pouch. A negative-pressure source may be fluidly coupled to the pouch. The negative-pressure source may have a first position and a second position. A first check valve may be fluidly coupled to the pouch and the negative-pressure source and operable to prevent fluid flow from the negative-pressure source into the pouch if the negative-pressure source is moved from the second position to the first position. A second check valve may be fluidly coupled to the negative-pressure source and the ambient environment and configured to prevent fluid flow from the ambient environment into the negative-pressure source if the negative-pressure source is moved from the first position to the second position.

A method for providing negative-pressure therapy to a tissue site is also described herein. A dressing assembly may be positioned adjacent to the tissue site. The dressing assembly may have an absorbent; a sealing layer configured to cover the absorbent; and a blister fluidly coupled to the absorbent. The blister may have a collapsed position and an expanded position. A first check valve may be fluidly coupled to the absorbent and the blister and configured to prevent fluid flow from the blister into the absorbent if the blister is moved from the expanded position to the collapsed position. A second check valve may be fluidly coupled to the blister and the ambient environment and configured to prevent fluid flow from the ambient environment into the blister if the blister is moved from the collapsed position to the expanded position. The blister may be compressed from the expanded position to the collapsed position to evacuate the blister. The blister may expand from the collapsed position to the expanded position to draw fluid from the absorbent.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a sectional view of an example embodiment of a negative-pressure therapy system that can provide negative-pressure therapy in accordance with this specification;

FIG. 2 is a top perspective view illustrating additional details that may be associated with an example embodiment of the negative-pressure therapy system of FIG. 1 in a first position;

FIG. 3 is a top perspective view illustrating additional details that may be associated with an example embodiment of the negative-pressure therapy system of FIG. 1 in a second position;

FIG. 4 is a sectional view of an example embodiment of another negative-pressure therapy system that can provide negative-pressure therapy in accordance with this specification;

FIG. 5 is a sectional view of an example embodiment of another negative-pressure therapy system that can provide negative-pressure therapy in accordance with this specification;

FIG. 6 is a top perspective view illustrating additional details that may be associated with an example embodiment of the negative-pressure therapy system of FIG. 5 in a first position;

FIG. 7 is a top perspective view illustrating additional details that may be associated with an example embodiment of the negative-pressure therapy system of FIG. 5 in a second position;

FIG. 8 is a sectional view of an example embodiment of another negative-pressure therapy system that can provide negative-pressure therapy in accordance with this specification;

FIG. 9 is a top perspective view illustrating additional details that may be associated with an example embodiment of the negative-pressure therapy system of FIG. 8:

FIG. 10 is a top perspective view illustrating additional details of another negative-pressure therapy system that can provide negative-pressure therapy in accordance with this specification;

FIG. 11 is a sectional view taken along line 11-11 of FIG. 10 illustrating additional details of the negative-pressure therapy system;

FIG. 12 is a bottom perspective view of a portion of the therapy system of FIG. 10 illustrating additional details that may be associated with some embodiments;

FIG. 13 is a sectional view illustrating additional details of another embodiment of the negative-pressure therapy system of FIG. 10;

FIG. 14 is a perspective view illustrating additional details of a testing apparatus that may be associated with some embodiments of the negative-pressure therapy system;

FIG. 15A is a line graph illustrating a load in Newtons (N) versus a deflection from preload in millimeters (mm) for a biasing member of the negative-pressure therapy system of FIG. 10;

FIG. 15B is a line graph illustrating a load in Newtons (N) versus a deflection from preload in millimeters (mm) for a free standing embodiment of the biasing member of FIG. 15A;

FIG. 15C is a line graph illustrating a pressure in millimeters mercury (mm Hg) versus time in minutes for the biasing member of FIG. 15A and FIG. 15B;

FIG. 16A is a line graph illustrating a load in Newtons (N) versus a deflection from preload in millimeters (mm) for a biasing member of the negative-pressure therapy system of FIG. 10;

FIG. 16B is a line graph illustrating a load in Newtons (N) versus a deflection from preload in millimeters (mm) for a free standing embodiment of the biasing member of FIG. 16A;

FIG. 16C is a line graph illustrating a pressure in millimeters mercury (mm Hg) versus time in minutes for the biasing member of FIG. 16A and FIG. 16B;

FIG. 17A is a line graph illustrating a load in Newtons (N) versus a deflection from preload in millimeters (mm) for a biasing member of the negative-pressure therapy system of FIG. 10;

FIG. 17B is a line graph illustrating a load in Newtons (N) versus a deflection from preload in millimeters (mm) for a free standing embodiment of the biasing member of FIG. 17A;

FIG. 17C is a line graph illustrating a pressure in millimeters mercury (mm Hg) versus time in minutes for the biasing member of FIG. 17A and FIG. 17B;

FIG. 18A is a line graph illustrating a load in Newtons (N) versus a deflection from preload in millimeters (mm) for a biasing member of the negative-pressure therapy system of FIG. 10;

FIG. 18B is a line graph illustrating a load in Newtons (N) versus a deflection from preload in millimeters (mm) for a free standing embodiment of the biasing member of FIG. 18A;

FIG. 18C is a line graph illustrating a pressure in millimeters mercury (mm Hg) versus time in minutes for the biasing member of FIG. 18A and FIG. 18B;

FIG. 19A is a line graph illustrating a load in Newtons (N) versus a deflection from preload in millimeters (mm) for a biasing member of the negative-pressure therapy system of FIG. 10;

FIG. 19B is a line graph illustrating a load in Newtons (N) versus a deflection from preload in millimeters (mm) for a free standing embodiment of the biasing member of FIG. 19A;

FIG. 19C is a line graph illustrating a pressure in millimeters mercury (mm Hg) versus time in minutes for the biasing member of FIG. 19A and FIG. 19B;

FIG. 20A is a line graph illustrating a load in Newtons (N) versus a deflection from preload in millimeters (mm) for a biasing member of the negative-pressure therapy system of FIG. 10;

FIG. 20B is a line graph illustrating a load in Newtons (N) versus a deflection from preload in millimeters (mm) for a free standing embodiment of the biasing member of FIG. 20A;

FIG. 20C is a line graph illustrating a pressure in millimeters mercury (mm Hg) versus time in minutes for the biasing member of FIG. 20A and FIG. 20B;

FIG. 21A is a line graph illustrating a load in Newtons (N) versus a deflection from preload in millimeters (mm) for a biasing member of the negative-pressure therapy system of FIG. 10;

FIG. 21B is a line graph illustrating a load in Newtons (N) versus a deflection from preload in millimeters (mm) for a free standing embodiment of the biasing member of FIG. 21A;

FIG. 21C is a line graph illustrating a pressure in millimeters mercury (mm Hg) versus time in minutes for the biasing member of FIG. 21A and FIG. 21B;

FIG. 22A is a line graph illustrating a load in Newtons (N) versus a deflection from preload in millimeters (mm) for a biasing member of the negative-pressure therapy system of FIG. 10;

FIG. 22B is a line graph illustrating a load in Newtons (N) versus a deflection from preload in millimeters (mm) for a free standing embodiment of the biasing member of FIG. 22A;

FIG. 22C is a line graph illustrating a pressure in millimeters mercury (mm Hg) versus time in minutes for the biasing member of FIG. 22A and FIG. 22B;

FIG. 23 is a perspective view illustrating additional details of a testing apparatus that may be associated with some embodiments of the negative-pressure therapy system;

FIG. 24A is a top view illustrating additional details of a biasing member that may be associated with some embodiments of the negative-pressure therapy system;

FIG. 24B is a top view illustrating additional details of a biasing member that may be associated with some embodiments of the negative-pressure therapy system;

FIG. 24C is a top view illustrating additional details of a biasing member that may be associated with some embodiments of the negative-pressure therapy system;

FIG. 25A is a line graph illustrating a load in Newtons (N) versus a deflection from preload in millimeters (mm) for a biasing member of the negative-pressure therapy system of FIG. 10;

FIG. 25B is a line graph illustrating a load in Newtons (N) versus a deflection from preload in millimeters (mm) for a biasing member of FIG. 25A; and

FIG. 25C is a line graph illustrating a load in Newtons (N) versus a deflection from preload in millimeters (mm) for a biasing member of the negative-pressure therapy system of FIG. 10.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

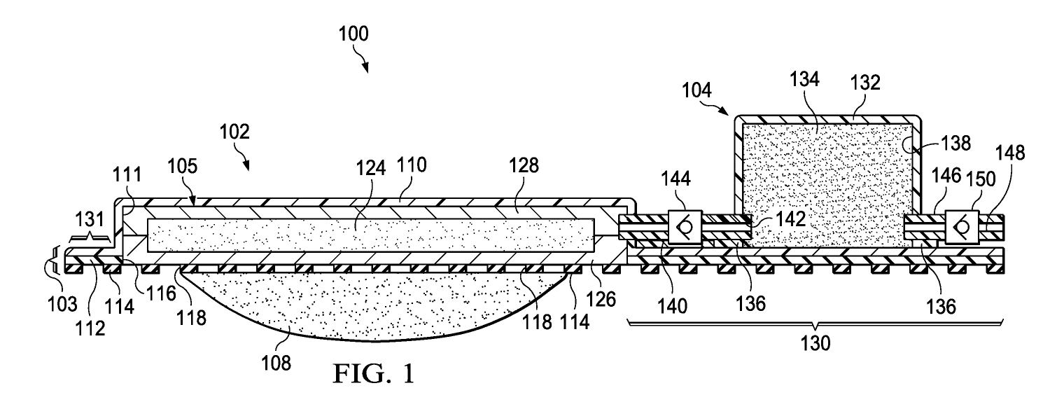

FIG. 1 is a sectional view of an example embodiment of a negative-pressure therapy system 100 that can provide negative-pressure therapy in accordance with this specification. The negative-pressure therapy system 100 may include a dressing assembly and a tissue interface. For example, a tissue interface 108 may be placed in a tissue site and a dressing assembly 102 may be placed over the tissue site and the tissue interface 108. The dressing assembly 102 may include a cover 103 and a pouch 105 which may be fluidly coupled to a negative-pressure source 104.

In general, components of the negative-pressure therapy system 100 may be coupled directly or indirectly. For example, the negative-pressure source 104 may be directly coupled to the pouch 105 and indirectly coupled to the tissue site through the pouch 105. Components may be fluidly coupled to each other to provide a path for transferring fluids (i.e., liquid and/or gas) between the components.

In some embodiments, components may be fluidly coupled through a tube, such as a tube 140 or a tube 146. A “tube,” as used herein, broadly refers to a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may additionally or alternatively be coupled by virtue of physical proximity, being integral to a single structure, or being formed from the same piece of material. Coupling may also include mechanical, thermal, electrical, or chemical coupling (such as a chemical bond) in some contexts.

In operation, the tissue interface 108 may be placed within, over, on, or otherwise proximate to a tissue site. The cover 103 may be placed over the tissue interface 108 and sealed to tissue near the tissue site. For example, the cover 103 may be sealed to undamaged epidermis peripheral to a tissue site. Thus, the dressing assembly 102 can provide a sealed therapeutic environment proximate to a tissue site, substantially isolated from the external environment, and the negative-pressure source 104 can reduce the pressure in the sealed therapeutic environment. The sealed therapeutic environment may be formed in the space occupied by the tissue interface 108 and the pouch 105. If the tissue interface 108 is not used, the sealed therapeutic environment may be formed in the space occupied by the pouch 105 and the tissue site. Negative pressure applied across the tissue site in the sealed therapeutic environment can induce macrostrain and microstrain in the tissue site, as well as remove exudates and other fluids from the tissue site, which can be collected in the pouch 105 and disposed of properly.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a negative-pressure source, and conversely, the term “upstream” implies a position relatively further away from a negative-pressure source. Similarly, it may be convenient to describe certain features in terms of fluid “inlet” or “outlet” in such a frame of reference. This orientation is generally presumed for purposes of describing various features and components of negative-pressure therapy systems herein. However, the fluid path may also be reversed in some applications (such as by substituting a positive-pressure source for a negative-pressure source) and this descriptive convention should not be construed as a limiting convention.

The term “tissue site” in this context broadly refers to a wound or defect located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be used in certain tissue areas to grow additional tissue that may be harvested and transplanted to another tissue location.

“Negative pressure” generally refers to a pressure less than a local ambient pressure, such as the ambient pressure in a local environment external to a sealed therapeutic environment provided by the dressing assembly 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites.

In some embodiments, the tissue interface 108 may be a manifold. A “manifold” in this context generally includes any substance or structure providing a plurality of pathways adapted to collect or distribute fluid across a tissue site under negative pressure. For example, a manifold may be adapted to receive negative pressure from a source and distribute the negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be channels that are interconnected to improve distribution or collection of fluids across a tissue site. For example, cellular foam, open-cell foam, reticulated foam, porous tissue collections, and other porous material such as gauze or felted mat generally include pores, edges, and/or walls adapted to form interconnected fluid pathways. Liquids, gels, and other foams may also include or be cured to include apertures and flow channels. In some illustrative embodiments, a manifold may be a porous foam material having interconnected cells or pores adapted to uniformly (or quasi-uniformly) distribute negative pressure to a tissue site. The foam material may be either hydrophobic or hydrophilic. In one non-limiting example, a manifold may be an open-cell, reticulated polyurethane foam such as V.A.C.® GRANUFOAM™ dressing available from Kinetic Concepts, Inc. of San Antonio, Tex.

In an example in which the tissue interface 108 may be made from a hydrophilic material, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. In some embodiments, the tissue interface 108 may be combined with hemostat material and anti-microbial materials to treat tissue sites that may have a significant depth.

In some embodiments, the cover 103 may be a sealing layer and provide a bacterial barrier and protection from physical trauma. The cover 103 may also be constructed from a material that can reduce evaporative losses and provide a fluid seal between two components or two environments, such as between a therapeutic environment and a local external environment. The cover 103 may be, for example, an elastomeric film or membrane that can provide a seal adequate to maintain a negative pressure at a tissue site for a given negative-pressure source. In some example embodiments, the cover 103 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 103 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 103 may be coated with an acrylic adhesive having a coating weight between 25-65 g.s.m. Thicker adhesives, or combinations of adhesives, may be applied in some embodiments, to improve the seal and reduce leaks. Other example embodiments of an attachment device may include a double-sided tape, paste, hydrocolloid, hydrogel, silicone gel, or organogel.

Typically, patients having low-acuity tissue sites may be mobile and may not require confinement to a care facility during the duration of the treatment of the tissue site. Consequently, a dedicated negative-pressure therapy system that requires a continuous supply of electrical current to provide negative-pressure therapy may not be preferable for use as a treatment device. Ambulatory patients may receive beneficial negative-pressure therapy by using the negative-pressure therapy system 100 described herein, which provides a peel-and-place dressing and negative-pressure source that allows the patient to easily see the status of the negative-pressure therapy and to reapply negative-pressure therapy without the intervention of a clinician.

As shown in FIG. 1, the negative-pressure therapy system 100 can include the tissue interface 108 and the dressing assembly 102 having the cover 103, the pouch 105, and the negative-pressure source 104. The cover 103, the pouch 105, and the negative-pressure source 104 may be coupled to each other and collectively placed over the tissue interface 108 and undamaged epidermis.

The pouch 105 may include an absorbent 124, a first outer layer, such as an upstream layer 126, and a second outer layer, such as a downstream layer 128. The upstream layer 126 and the downstream layer 128 may envelop or enclose the absorbent 124. The absorbent 124 may hold, stabilize, and/or solidify fluids collected from the tissue site. The absorbent 124 may be formed from materials referred to as “hydrogels,” “super-absorbents,” or “hydrocolloids.” If disposed within the dressing assembly 102, the absorbent 124 may be formed into fibers or spheres to manifold negative pressure until the absorbent 124 becomes saturated. Spaces or voids between the fibers or spheres may allow a negative pressure that is supplied to the dressing assembly 102 to be transferred within and through the absorbent 124 to the tissue interface 108 and the tissue site. In some exemplary embodiments, the absorbent 124 may be Texsus FP2325 having a material density of about 800 grams per square meter (gsm). In other exemplary embodiments, the absorbent material may be BASF 402C, Technical Absorbents 2317 available from Technical Absorbents (www.techabsorbents.com), sodium polyacrylate super absorbers, cellulosics (carboxy methyl cellulose and salts such as sodium CMC), or alginates.

In some exemplary embodiments, the absorbent 124 may be formed of granular absorbent components that may be scatter coated onto a paper substrate. Scatter coating involves spreading a granular absorbent powder uniformly onto a textile substrate, such as paper. The substrate, having the granular absorbent powder disposed thereon, may be passed through an oven to cure the powder and cause the powder to adhere to the paper substrate. The cured granular absorbent powder and substrate may be passed through a calender machine to provide a smooth uniform surface to the absorbent material.

In some exemplary embodiments, the upstream layer 126 and the downstream layer 128 have perimeter dimensions that may be larger than the perimeter dimensions of the absorbent 124 so that, if the absorbent 124 is positioned between the upstream layer 126 and the downstream layer 128 and the center portions of the absorbent 124, the upstream layer 126, and the downstream layer 128 are aligned, the upstream layer 126 and the downstream layer 128 may extend beyond the perimeter of the absorbent 124. In some exemplary embodiments, the upstream layer 126 and the downstream layer 128 surround the absorbent 124. Peripheral portions of the upstream layer 126 and the downstream layer 128 may be coupled so that the upstream layer 126 and the downstream layer 128 enclose the absorbent 124. The upstream layer 126 and the downstream layer 128 may be coupled by high frequency welding, ultrasonic welding, heat welding, or impulse welding, for example. In other exemplary embodiments, the upstream layer 126 and the downstream layer 128 may be coupled by bonding or folding, for example.

The upstream layer 126 may be formed of non-woven material in some embodiments. For example, the upstream layer 126 may have a polyester fibrous porous structure. The upstream layer 126 may be porous, but preferably the upstream layer 126 is not perforated. The upstream layer 126 may have a material density between about 80 gsm and about 150 gsm. In other exemplary embodiments, the material density may be lower or greater depending on the particular application of the pouch 105. In some embodiments, the upstream layer 126 may be a plurality of layers of non-woven material. The upstream layer 126 may be formed of Libeltex TDL2, for example. In other embodiments, the upstream layer 126 may also be formed of Libeltex TL4.

The downstream layer 128 may also be formed of a non-woven material in some embodiments. For example, the downstream layer 128 may have a polyester fibrous porous structure. The downstream layer 128 may be porous, but the downstream layer 128 preferably is not perforated. The downstream layer 128 may have a material density between about 80 gsm and about 150 gsm. In other exemplary embodiments, the material density may be lower or greater depending on the particular application of the pouch 105. The material density of the downstream layer 128 may be greater or less than the material density of the upstream layer 126. In some embodiments, a thickness of the downstream layer 128 may be greater than a thickness of the upstream layer 126. In other embodiments, the thickness of the downstream layer 128 may be less than the thickness of the upstream layer 126. In some embodiments, the downstream layer 128 may be a plurality of layers of non-woven material. The downstream layer 128 may be formed of Libeltex TL4. In other exemplary embodiments, the downstream layer 128 may be formed of Libeltex TDL2.

The upstream layer 126 and the downstream layer 128 may be manifolding layers configured to facilitate fluid movement through the pouch 105. In some embodiments, the upstream layer 126 and the downstream layer 128 may each have a hydrophobic side and a hydrophilic side. The hydrophobic side may also be referred to as a wicking side, wicking surface, distribution surface, distribution side, or fluid distribution surface. The hydrophobic side may be a smooth distribution surface configured to move fluid along a grain of the upstream layer 126 and the downstream layer 128, distributing fluid throughout the upstream layer 126 and the downstream layer 128. The hydrophilic side may be configured to acquire bodily fluid from the hydrophobic side to aid in bodily fluid movement into the absorbent 124. The hydrophilic side may also be referred to as a fluid acquisition surface, fluid acquisition side, hydrophilic acquisition surface, or hydrophilic acquisition side. The hydrophilic side may be a fibrous surface and be configured to draw fluid into the upstream layer 126 and the downstream layer 128. In some embodiments, the hydrophilic side of the upstream layer 126 and the downstream layer 128 may be positioned adjacent to the absorbent 124. In other embodiments, the hydrophobic side of the upstream layer 126 and the downstream layer 128 may be positioned adjacent to the absorbent 124. In still other embodiments, the hydrophilic side of one of the upstream layer 126 or the downstream layer 128 may be positioned adjacent to the absorbent 124, and the hydrophobic side of the other of the upstream layer 126 or the downstream layer 128 may be positioned adjacent to the absorbent 124.

In some embodiments, the cover 103 may include or may be a hybrid drape having a barrier layer 110, a bonding adhesive layer 112, and a sealing adhesive layer 114. The barrier layer 110 may be formed from a range of medically approved films ranging in thickness from about 15 microns (m) to about 50 microns (m). The barrier layer 110 may comprise a suitable material or materials, such as the following: hydrophilic polyurethane (PU), cellulosics, hydrophilic polyamides, polyvinyl alcohol, polyvinyl pyrrolidone, hydrophilic acrylics, hydrophilic silicone elastomers, and copolymers of these. In some embodiments, the barrier layer 110 may be formed from a breathable cast matt polyurethane film sold by Transcontinental Advanced Coatings of Wrexham, United Kingdom, under the name INSPIRE 2301.

The barrier layer 110 may have a high moisture vapor transmission rate (MVTR). The MVTR of the barrier layer 110 allows vapor to egress and inhibits liquids from exiting. In some embodiments, the MVTR of the barrier layer 110 may be greater than or equal to 300 g/m²/24 hours. In other embodiments, the MVTR of the barrier layer 110 may be greater than or equal to 1000 g/m²/24 hours. The illustrative INSPIRE 2301 film may have an MVTR (inverted cup technique) of 14400 g/m²/24 hours and may be approximately 30 microns thick. In other embodiments, a drape having a low MVTR or that allows no vapor transfer might be used. The barrier layer 110 can also function as a barrier to liquids and microorganisms.

In some embodiments, the barrier layer 110 may be adapted to form a bulge on a first side of the barrier layer and a cavity 111 on an opposite side of the barrier layer from the bulge. For example, the barrier layer 110 may be placed on a mold and stretched to plastically deform a portion of the barrier layer 110, forming the cavity 111. A periphery of the barrier layer 110 that is not stretched by the formation of the cavity 111 may form a flange surrounding the cavity 111. In some embodiments, the cavity 111 may be positioned so that a portion of the flange may be larger on a first side of the cavity 111 than on a second side of the cavity 111. The disparity in sizes of the flange may form a foundational flange 130 and a sealing flange 131. In some embodiments, the pouch 105 may be disposed in the cavity 111. The cavity 111 may also be a portion of the barrier layer 110 that is free of adhesive. For example, during manufacturing, a portion of the barrier layer 110 may be left without the bonding adhesive layer 112; the area of the barrier layer 110 without the bonding adhesive layer 112 may be equal to a surface area of the pouch 105 to be covered by the barrier layer 110.

The foundational flange 130 may extend away from the cavity 111. In some embodiments, the foundational flange 130 may have a length and a width sufficient to permit other objects to be coupled to the dressing assembly 102. For example, the foundational flange 130 may support the negative-pressure source 104, as illustrated in FIG. 1.

The bonding adhesive layer 112 may be coupled to the barrier layer 110 on a side of the barrier layer 110 having an opening of the cavity 111. In some embodiments, the bonding adhesive layer 112 may include an aperture 116. The aperture 116 may be coextensive with the opening of the cavity 111. For example, the bonding adhesive layer 112 may cover the barrier layer 110 at the foundational flange 130 and the sealing flange 131, leaving the portion of the barrier layer 110 forming the cavity 111 free of the bonding adhesive layer 112.

The bonding adhesive layer 112 may comprise an acrylic adhesive, rubber adhesive, high-tack silicone adhesive, polyurethane, or other substance. In an illustrative example, the bonding adhesive layer 112 comprises an acrylic adhesive with coating weight of 15 grams/m² (gsm) to 70 grams/m² (gsm). The bonding adhesive layer 112 may be a continuous layer of material or may be a layer with apertures (not shown). The apertures may be formed after application of the bonding adhesive layer 112 or may be formed by coating the bonding adhesive layer 112 in patterns on a carrier layer. In some embodiments, the bond strength of the bonding adhesive may have a peel adhesion or resistance to being peeled from a stainless steel material between about 6N/25 mm to about 10N/25 mm on stainless steel substrate at 23° C. at 50% relative humidity based on the American Society for Testing and Materials (“ASTM”) standard ASTM D3330. The bonding adhesive layer 112 may be about 30 microns to about 60 microns in thickness.

The sealing adhesive layer 114 may be coupled to the bonding adhesive layer 112 and the pouch 105. For example, the sealing adhesive layer 114 may cover the sealing flange 131, the pouch 105, and the foundational flange 130. The sealing adhesive layer 114 may be formed with the plurality of apertures 118. The apertures 118 may be numerous shapes, for example, circles, squares, stars, ovals, polygons, slits, complex curves, rectilinear shapes, triangles, or other shapes. Each aperture 118 of the plurality of apertures 118 may have an effective diameter, which is the diameter of a circular area having the same surface area as the aperture 118. The average effective diameter of each aperture 118 may typically be in the range of about 6 mm to about 50 mm. The plurality of apertures 118 may have a uniform pattern or may be randomly distributed in the sealing adhesive layer 114. Generally, the apertures 118 may be disposed across a length and width of the sealing adhesive layer 114.

The sealing adhesive layer 114 may comprise a silicone gel (or soft silicone), hydrocolloid, hydrogel, polyurethane gel, polyolefin gel, hydrogenated styrenic copolymer gels, or foamed gels with compositions as listed, or soft closed cell foams (polyurethanes, polyolefins) coated with an adhesive (e.g., 30 gsm-70 gsm acrylic), polyurethane, polyolefin, or hydrogenated styrenic copolymers. The sealing adhesive layer 114 may have a thickness in the range of about 100 microns (m) to about 1000 microns (m). In some embodiments, the sealing adhesive layer 114 may have stiffness between about 5 Shore 00 and about 80 Shore 00. The sealing adhesive layer 114 may be hydrophobic or hydrophilic. The sealing adhesive of the sealing adhesive layer 114 may be an adhesive having a low to medium tackiness, for example, a silicone polymer, polyurethane, or an additional acrylic adhesive. In some embodiments, the bond strength of the sealing adhesive may have a peel adhesion or resistance to being peeled from a stainless steel material between about 0.5N/25 mm to about 1.5N/25 mm on stainless steel substrate at 23° C. at 50% relative humidity based on ASTM D3330. The sealing adhesive may have a tackiness such that the sealing adhesive may achieve the bond strength above after a contact time of less than about 60 seconds. Tackiness may be considered a bond strength of an adhesive after a very low contact time between the adhesive and a substrate. In some embodiments, the sealing adhesive layer 114 may have a tackiness that may be about 30% to about 50% of the tackiness of the bonding adhesive of the bonding adhesive layer 112.

In the assembled state, the bonding adhesive layer 112 may be coupled to the barrier layer 110. The sealing adhesive layer 114 may be coupled to the bonding adhesive layer 112 at the sealing flange 131 and the foundational flange 130 and to the pouch 105 at the cavity 111. In some embodiments, a scrim layer may be disposed in the sealing adhesive layer 114. The scrim layer may provide additional mechanical support for the sealing adhesive layer 114. In some embodiments, the sealing adhesive layer 114 may be treated on a portion and a side of the sealing adhesive layer 114 adjacent to the pouch 105. The treated portion of the sealing adhesive layer 114 may reduce the tackiness of the sealing adhesive layer 114 so that the sealing adhesive layer 114 may not readily adhere to the pouch 105. The initial tackiness of the sealing adhesive layer 114 is preferably sufficient to initially couple the sealing adhesive layer 114 to the epidermis by forming sealing couplings. Once in the desired location, a force can be applied to the barrier layer 110 of the cover 103. For example, the user may rub the foundational flange 130 and the sealing flange 131. This action can cause at least a portion of the bonding adhesive layer 112 to be forced into the plurality of apertures 118 and into contact with the epidermis to form bonding couplings. The bonding couplings provide secure, releasable mechanical fixation to the epidermis.

The average effective diameter of the plurality of apertures 118 for the sealing adhesive layer 114 may be varied as one control of the tackiness or adhesion strength of the cover 103. In this regard, there is interplay between three main variables for each embodiment: the thickness of the sealing adhesive layer 114, the average effective diameter of the plurality of apertures 118, and the tackiness of the bonding adhesive layer 112. The more bonding adhesive of the bonding adhesive layer 112 that extends through the apertures 118, the stronger the bond of the bonding coupling. The thinner the sealing adhesive layer 114, the more bonding adhesive of the bonding adhesive layer 112 generally extends through the apertures 118 and the greater the bond of the bonding coupling. As an example of the interplay, if a very tacky bonding adhesive layer 112 is used and the thickness of the sealing adhesive layer 114 is small, the average effective diameter of the plurality of apertures 118 may be relatively smaller than if the bonding adhesive layer 112 is less tacky and the sealing adhesive layer 114 is thicker. In some embodiments, the thickness of the sealing adhesive layer 114 may be approximately 200 microns, the thickness of the bonding adhesive layer 112 may be approximately 30 microns with a tackiness of 2000 g/25 cm wide strip, and the average effective diameter of each aperture 118 may be approximately 6 mm.

As illustrated in FIG. 1, the negative-pressure source 104, which may also be referred to as a blister, may be coupled to the barrier layer 110 of the foundational flange 130. The negative-pressure source 104 may include a barrier layer and a biasing member, for example, a film layer 132 and a foam block 134. In some embodiments, the film layer 132 may form a source flange 136 and a source cavity 138. The source cavity 138 may be a portion of the film layer 132 that is plastically deformed, such as by vacuum forming, thermoforming, micro-thermoforming, injection molding, or blow molding, for example. In some embodiments, the source cavity 138 may form walls of the negative-pressure source 104 that may be resilient or flexible. The source flange 136 may be a portion of the film layer 132 adjacent to and surrounding an opening of the source cavity 138. In some embodiments, the foam block 134 may be disposed in the source cavity 138. The source flange 136 may be coupled to the barrier layer 110 of the foundational flange 130 to seal the foam block 134 in the source cavity 138. In some embodiments, the source flange 136 may be coupled to the barrier layer 110 by high frequency welding, ultrasonic welding, heat welding, or impulse welding, for example. In other exemplary embodiments, the source flange 136 may be coupled to the barrier layer 110 by bonding or folding, for example. In some embodiments, if the source flange 136 is coupled to the barrier layer 110 of the foundational flange 130, the source cavity 138 may be fluidly isolated from the ambient environment and the pouch 105.

The film layer 132 may be constructed from a material that can provide a fluid seal between two components or two environments, such as between the source cavity 138 and a local external environment, while allowing for repeated elastic deformation of the film layer 132. The film layer 132 may be, for example, an elastomeric film or membrane that can provide a seal between the source cavity 138 and the ambient environment. In some example embodiments, the film layer 132 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. In an exemplary embodiment, the film layer 132 may be a polyurethane having a thickness between about 50 microns and about 250 microns and preferably about 100 microns.

The foam block 134 may be a foam having a plurality of interconnected flow channels. For example, cellular foam, open-cell foam, reticulated foam, porous tissue collections, and other porous material that generally include pores, edges, and/or walls adapted to form interconnected fluid pathways. Liquids, gels, and other foams may also include or be cured to include apertures and flow channels. In some illustrative embodiments, the foam block 134 may be a porous foam material having interconnected cells or pores adapted to uniformly (or quasi-uniformly) distribute fluid throughout the foam block 134. The foam material may be either hydrophobic or hydrophilic. In one non-limiting example, the foam block 134 may be an open-cell, reticulated polyurethane foam such as V.A.C.® GRANUFOAM™ dressing available from Kinetic Concepts, Inc. of San Antonio, Tex. Another exemplary embodiment of the foam block 134 may be Z48AA foam from FXI®. In some embodiments, the foam block 134 may include an indicator, such as a color change dye. The indicator may change colors if contacted by a liquid. Consequently, if the foam block 134 changes colors, a user may know that the dressing assembly 102 is saturated.

Foam materials may have an elastic modulus, which may also be referred to as a foam modulus. Generally, the elastic modulus of a material may measure the resistance of the material to elastic deformation under a load. The elastic modulus of a material may be defined as the slope of a stress-strain curve in the elastic deformation region of the curve. The elastic deformation region of a stress-strain curve represents that portion of the curve where a deformation of a material due to an applied load is elastic, that is, not permanent. If the load is removed, the material may return to its preloaded state. Stiffer materials may have a higher elastic modulus, and more compliant materials may have a lower elastic modulus. Generally, references to the elastic modulus of a material refers to a material under tension.

For some materials under compression, the elastic modulus can be compared between materials by comparing the compression force deflection (CFD) of the materials. Typically, CFD is determined experimentally by compressing a sample of a material until the sample is reduced to about 25% of its uncompressed size. The load applied to reach the 25% compression of the sample is then divided by the area of the sample over which the load is applied to arrive at the CFD. The CFD can also be measured by compressing a sample of a material to about 50% of the sample's uncompressed size. The CFD of a foam material can be a function of compression level, polymer stiffness, cell structure, foam density, and cell pore size. In some embodiments, the foam block 134 may have a CFD that is greater than a CFD of the tissue interface 108. For example, the tissue interface 108 may have a 25% CFD of about 2 kPa. The tissue interface 108 may compress to about 25% of its uncompressed size if a load of about 2 kPa is applied to the tissue interface 108. The foam block 134 may have a CFD of about 4 kPA. The foam block 134 may compress to about 25% of its uncompressed size if a load of about 4 kPa is applied to the foam block 134. Thus, the foam block 134 is more resistant to deformation than the tissue interface 108.

Furthermore, CFD can represent the tendency of a foam to return to its uncompressed state if a load is applied to compress the foam. For example, a foam having a CFD of about 4 kPa may exert about 4 kPa in reaction to 25% compression. The CFD of the foam block 134 may represent the ability of the foam block 134 to bias the film layer 132 toward an expanded position. For example, if the foam block 134 is compressed to 25% of its original size, the foam block 134 may exert a spring force that opposes the applied force over the area of the foam block 134 to which the force is applied. The reactive force may be proportional to the amount the foam block 134 is compressed.

The foam block 134 may have a free volume. The free volume of the foam block 134 may be the volume of free space of the foam block 134, for example, the volume of the plurality of channels of the foam block 134. In some embodiments, the free volume of the foam block 134 may be greater than the free volume of the sealed therapeutic environment. For example, the free volume of the foam block 134 may be greater than the free volume of the pouch 105. If the tissue interface 108 is used with the dressing assembly 102, the free volume of the foam block 134 may be greater than the combined free volume of the pouch 105 and the tissue interface 108. For example, if the free volume of the pouch 105 is 10 cm³ and the free volume of the tissue interface is 10 cm³, then the free volume of the foam block 134 may be greater than about 20 cm³.

In some embodiments, the negative-pressure source 104 may be fluidly coupled to the cavity 111 through a fluid inlet, such as the tube 140. The tube 140 may be representative of a fluid communication path between the negative-pressure source 104 and the cavity 111. In other embodiments, the tube 140 may be a sealed channel or other fluid pathway. The tube 140 may include a lumen 142 fluidly coupled to the source cavity 138 and the pouch 105. In some embodiments, a valve, such as a check valve 144, may be fluidly coupled to the lumen 142. Exemplary check valves 144 may include ball check valves, diaphragm check valves, swing check valves, stop-check valves, duckbill valves, or pneumatic non-return valves. The check valve 144 may permit fluid communication from the pouch 105 to the source cavity 138 and prevent fluid communication from the source cavity 138 to the pouch 105. For example, if a pressure in the pouch 105 is greater than a pressure in the source cavity 138, the check valve 144 may open, and if the pressure in the source cavity 138 is greater than the pressure in the pouch 105, the check valve 144 may close.

In some embodiments, a filter may be disposed on an end of the tube 140. The filter may be a hydrophobic porous polymer filter having gel blocking properties. In some embodiments, the filter may be a non-gel blocking filter, such as a Gore MMT314 material having a polytetrafluoroethylene (PTFE) layer. The PTFE layer may face the manifolding structure to prevent fluid communication across the PTFE layer. In some embodiments, the filter may be on an end of the tube 140 proximate to the dressing assembly 102. In other embodiments, the filter may be on an end of the tube 140 proximate to the negative-pressure source 104.

The source cavity 138 may also be fluidly coupled to the ambient environment through a fluid outlet, such as the tube 146. For example, the tube 146 having a lumen 148 may fluidly couple the source cavity 138 to the ambient environment. The tube 146 may be representative of a fluid communication path between the ambient environment and the source cavity 138. A valve, such as a check valve 150, may be fluidly coupled to the lumen 148 to control fluid communication through the lumen 148. Exemplary check valves 150 may include ball check valves, diaphragm check valves, swing check valves, stop-check valves, duckbill valves, or pneumatic non-return valves. In some embodiments, the check valve 150 may permit fluid communication from the source cavity 138 to the ambient environment and prevent fluid communication from the ambient environment to the source cavity 138. For example, if a pressure in the source cavity 138 is greater than a pressure in the ambient environment, the check valve 150 may open, and if the pressure in the ambient environment is greater than the pressure in the source cavity 138, the check valve 150 may close.

In some embodiments, a filter may be disposed on an end of the tube 146. The filter may be a hydrophobic porous polymer filter having gel blocking properties. In some embodiments, the filter may be a non-gel blocking filter, such as a Gore MMT314 material having a polytetrafluoroethylene (PTFE) layer. The PTFE layer may face the manifolding structure to prevent fluid communication across the PTFE layer. In some embodiments, the filter may be on an end of the tube 146 proximate to the negative-pressure source 104. In other embodiments, the filter may be on an end of the tube 140 proximate to the ambient environment.

In some embodiments, the tissue interface 108 may be disposed adjacent to a tissue site. If the tissue interface 108 is used, the thickness of the tissue interface 108 may preferably be less than about 10 mm. The dressing assembly 102 may be disposed over the tissue interface 108 to create the sealed therapeutic environment. In some embodiments, the pouch 105 of the dressing assembly 102 may be positioned over the tissue interface 108 and the negative-pressure source 104 may be positioned over undamaged tissue proximate the tissue interface 108. A force, such as hand pressure, may be applied to the sealing flange 131 and the foundational flange 130, urging the bonding adhesive of the bonding adhesive layer 112 through the apertures 118 of the sealing adhesive layer 114 to form bonding couplings and securing the negative-pressure therapy system 100 to the tissue site.

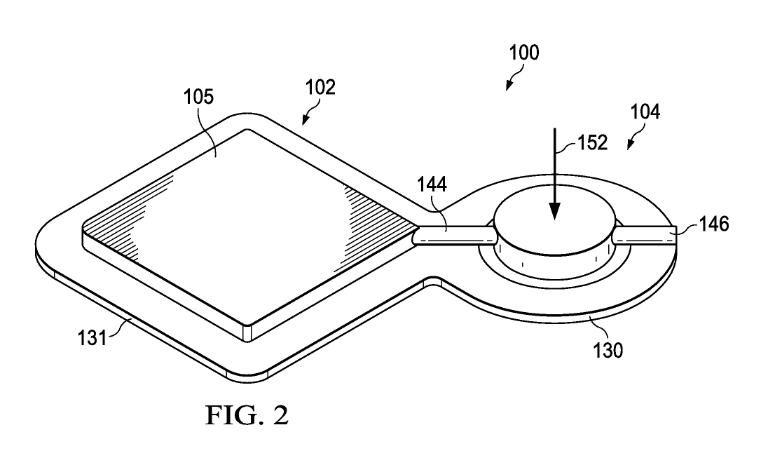

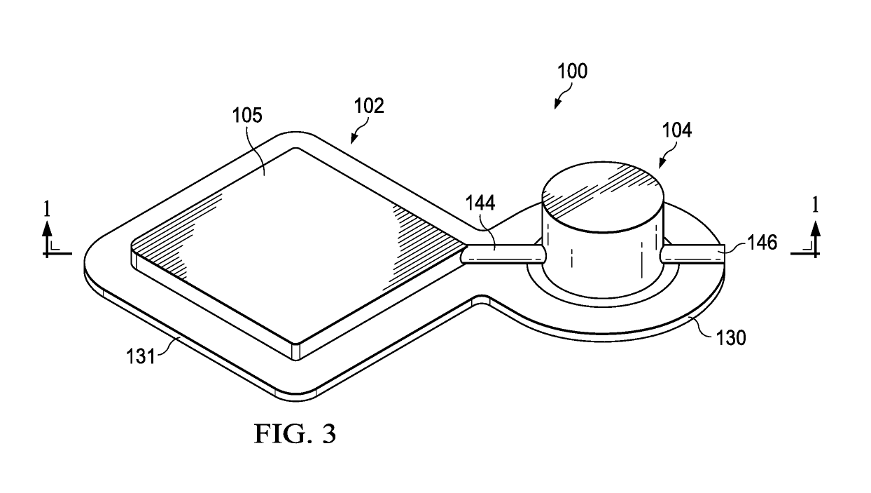

FIG. 2 is a perspective view illustrating additional details of the negative-pressure source 104 in a first position, such as a collapsed position, and FIG. 3 is a perspective view illustrating additional details of the negative-pressure source 104 is a second position, such as an expanded position. Once positioned, the negative-pressure source 104 may be operated to generate a negative pressure in the pouch 105. As shown in FIG. 2, a force 152, such as hand pressure, may be applied to the film layer 132 over the foam block 134 to compress the foam block 134 to the first position and decrease the volume of the source cavity 138. If the foam block 134 and the source cavity 138 are fluidly isolated from the ambient environment, compression of the foam block 134 may increase the pressure in the source cavity 138. An increase of pressure in the source cavity 138 may create a pressure differential across the check valve 144 that urges the check valve 144 to close. Similarly, an increase of pressure in the source cavity 138 may create a pressure differential across the check valve 150 that urges the check valve 150 to open, allowing fluid from the source cavity 138 to flow through the tube 146 to the ambient environment. If the force 152 is removed, the foam block 134 may expand, increasing the volume of the source cavity 138 and decreasing the pressure in the source cavity 138. In response, the decrease in pressure in the source cavity 138 may create a pressure differential across the check valve 150 that urges the check valve 150 to close, preventing fluid from flowing from the ambient environment to the source cavity 138. The decrease in pressure in the source cavity 138 may also create a pressure differential across the check valve 144 that urges the check valve 144 to open, permitting fluid flow from the pouch 105 to the source cavity 138. Fluid may flow from the pouch 105 to the source cavity 138 until the source cavity 138 and the foam block 134 reach their respective uncompressed positions as shown in FIG. 3. In this manner, a portion of the total volume of fluid in the sealed therapeutic environment may be removed. In response to the removal of a portion of the fluid, a smaller volume of fluid occupies the sealed therapeutic environment, decreasing the pressure in the sealed therapeutic environment. Each time the foam block 134 is compressed and allowed to rebound, additional fluid may be removed from the sealed therapeutic environment, further decreasing the pressure.

Decreasing the pressure in the sealed therapeutic environment may create a pressure differential across the dressing assembly 102. If the pressure in the sealed therapeutic environment reaches the therapy pressure for negative-pressure therapy, the CFD of the foam block 134 may be insufficient to cause the foam block 134 to expand following compression of the foam block 134 from the second position of FIG. 3 to the first position of FIG. 2. The therapy pressure may be the pressure at which negative-pressure therapy may be performed. In some embodiments, the therapy pressure provided by the foam block 134 may be about 70 mm Hg of negative pressure. In other embodiments, the therapy pressure provided by the foam block 134 may be between about 50 mm Hg and 150 mm Hg of negative pressure. If the foam block 134 remains compressed as shown in FIG. 2, a patient or clinician may have an indication that the therapy pressure has been reached. The compressed foam block 134 may also act as a pressure reservoir. As negative-pressure therapy is provided, there may be a natural leakage or decline of negative pressure at the tissue site. As the negative pressure decreases in the sealed therapeutic environment, the pressure differential across the dressing assembly 102 may decrease and the foam block 134 may gradually expand, reapplying negative pressure at the tissue site. In some embodiments, the negative-pressure source 104 having the foam block 134 may maintain a therapeutic negative pressure for about 8 hours or more.

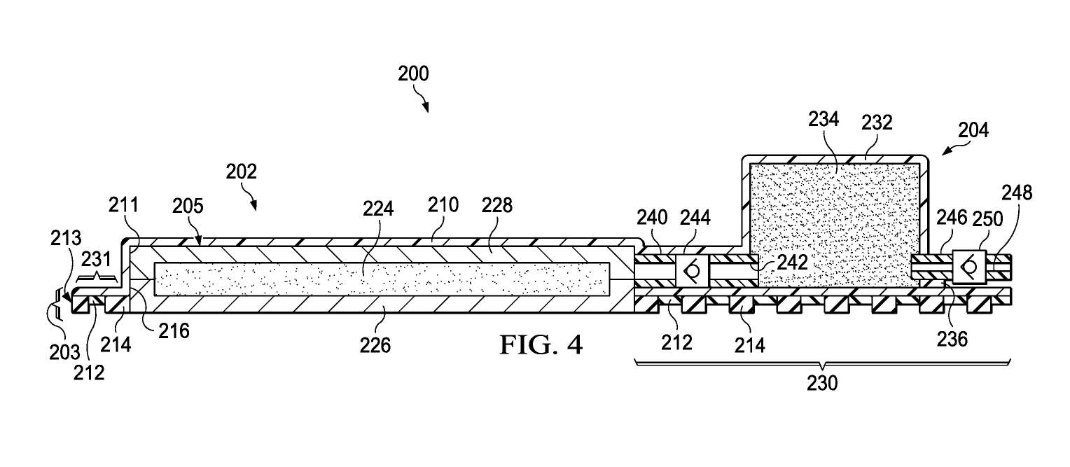

FIG. 4 is a sectional view of an example embodiment of a negative-pressure therapy system 200 that can provide negative-pressure therapy in accordance with this specification. The negative-pressure therapy system 200 may be similar to and operate as described above with respect to the negative-pressure therapy system 100. Similar elements have similar reference numbers indexed to 200. As shown in FIG. 4, the negative-pressure therapy system 200 can include a dressing assembly 202 having a cover 203, a pouch 205, and a negative-pressure source 204. The cover 203, the pouch 205, and the negative-pressure source 204 may be coupled to each other. In some embodiments, the negative-pressure therapy system 200 can also include the tissue interface 108.

The pouch 205 may include an absorbent 224, a first outer layer, such as an upstream layer 226, and a second outer layer, such as a downstream layer 228. The upstream layer 226 and the downstream layer 228 may envelop or enclose the absorbent 224. The absorbent 224 may hold, stabilize, and/or solidify fluids that may be collected from the tissue site. The absorbent 224 may be of the type referred to as “hydrogels,” “super-absorbents,” or “hydrocolloids.” If disposed within the dressing assembly 202, the absorbent 224 may be formed into fibers or spheres to manifold negative pressure until the absorbent 224 becomes saturated. Spaces or voids between the fibers or spheres may allow a negative pressure that is supplied to the dressing assembly 202 to be transferred within and through the absorbent 224 to the tissue site. In some exemplary embodiments, the absorbent 224 may be Texsus FP2325 having a material density of about 800 grams per square meter (gsm). In other exemplary embodiments, the absorbent material may be BASF 402C, Technical Absorbents 2317 available from Technical Absorbents (www.techabsorbents.com), sodium polyacrylate super absorbers, cellulosics (carboxy methyl cellulose and salts such as sodium CMC), or alginates.

In some exemplary embodiments, the absorbent 224 may be formed of granular absorbent components that may be scatter coated onto a paper substrate. Scatter coating involves spreading a granular absorbent powder uniformly onto a textile substrate, such as paper. The substrate, having the granular absorbent powder disposed thereon, may be passed through an oven to cure the powder and cause the powder to adhere to the paper substrate. The cured granular absorbent powder and substrate may be passed through a calender machine to provide a smooth uniform surface to the absorbent material.

In some exemplary embodiments, the upstream layer 226 and the downstream layer 228 have perimeter dimensions that may be larger than the perimeter dimensions of the absorbent 224 so that, if the absorbent 224 is positioned between the upstream layer 226 and the downstream layer 228 and the center portions of the absorbent 224, the upstream layer 226, and the downstream layer 228 are aligned, the upstream layer 226 and the downstream layer 228 may extend beyond the perimeter of the absorbent 224. In some exemplary embodiments, the upstream layer 226 and the downstream layer 228 surround the absorbent 224. Peripheral portions of the upstream layer 226 and the downstream layer 228 may be coupled so that the upstream layer 226 and the downstream layer 228 enclose the absorbent 224. The upstream layer 226 and the downstream layer 228 may be coupled by high frequency welding, ultrasonic welding, heat welding, or impulse welding, for example. In other exemplary embodiments, the upstream layer 226 and the downstream layer 228 may be coupled by bonding or folding, for example.

The upstream layer 226 may be formed of non-woven material in some embodiments. For example, the upstream layer 226 may have a polyester fibrous porous structure. The upstream layer 226 may be porous, but preferably the upstream layer 226 is not perforated. The upstream layer 226 may have a material density between about 80 gsm and about 150 gsm. In other exemplary embodiments, the material density may be lower or greater depending on the particular application of the pouch 205. In some embodiments, the upstream layer 226 may a plurality of layers of, for example, non-woven material. The upstream layer 226 may be formed of Libeltex TDL2, for example. In other embodiments, the upstream layer 226 may also be formed of Libeltex TL4. The upstream layer 226 may have a hydrophilic side and a hydrophobic side.

The downstream layer 228 may also be formed of a non-woven material in some embodiments. For example, the downstream layer 228 may have a polyester fibrous porous structure. The downstream layer 228 may be porous, but the downstream layer 228 preferably is not perforated. The downstream layer 228 may have a material density between about 80 gsm and about 150 gsm. In other exemplary embodiments, the material density may be lower or greater depending on the particular application of the pouch 205. The material density of the downstream layer 228 may be greater or less than the material density of the upstream layer 226. In some embodiments, a thickness of the downstream layer 228 may be greater than a thickness of the upstream layer 226. In other embodiments, the thickness of the downstream layer 228 may be less than the thickness of the upstream layer 226. In some embodiments, the downstream layer 228 may a plurality of layers of, for example, non-woven material. The downstream layer 228 may be formed of Libeltex TL4. In other exemplary embodiments, the downstream layer 228 may be formed of Libeltex TDL2.

The upstream layer 226 and the downstream layer 228 may be manifolding layers configured to facilitate fluid movement through the pouch 205. In some embodiments, the upstream layer 226 and the downstream layer 228 may each have a hydrophobic side and a hydrophilic side. The hydrophobic side may also be referred to as a wicking side, wicking surface, distribution surface, distribution side, or fluid distribution surface. The hydrophobic side may be a smooth distribution surface configured to move fluid along a grain of the upstream layer 226 and the downstream layer 228, distributing fluid throughout the upstream layer 226 and the downstream layer 228. The hydrophilic side may be configured to acquire bodily fluid from the hydrophobic side to aid in bodily fluid movement into the absorbent 224. The hydrophilic side may also be referred to as a fluid acquisition surface, fluid acquisition side, hydrophilic acquisition surface, or hydrophilic acquisition side. The hydrophilic side may be a fibrous surface and be configured to draw fluid into the upstream layer 226 and the downstream layer 228. In some embodiments, the hydrophilic side of the upstream layer 226 and the downstream layer 228 may be positioned adjacent to the absorbent 224. In other embodiments, the hydrophobic side of the upstream layer 226 and the downstream layer 228 may be positioned adjacent to the absorbent 224. In still other embodiments, the hydrophilic side of one of the upstream layer 226 or the downstream layer 228 may be positioned adjacent to the absorbent 224, and the hydrophobic side of the other of the upstream layer 226 or the downstream layer 228 may be positioned adjacent to the absorbent 224.

In some embodiments, the cover 203 may include a barrier layer 210 and an adhesive layer 213 having a bonding adhesive 212 and a sealing adhesive 214. The barrier layer 210 may be formed from a range of medically approved films ranging in thickness from about 15 microns (m) to about 50 microns (m). The barrier layer 210 may comprise a suitable material or materials, such as the following: hydrophilic polyurethane (PU), cellulosics, hydrophilic polyamides, polyvinyl alcohol, polyvinyl pyrrolidone, hydrophilic acrylics, hydrophilic silicone elastomers, and copolymers of these. In some embodiments, the barrier layer 210 may be formed from a breathable cast matt polyurethane film sold by Transcontinental Advanced Coatings of Wrexham, United Kingdom, under the name INSPIRE 2301.

The barrier layer 210 may have a high moisture vapor transmission rate (MVTR). The MVTR of the barrier layer 210 allows vapor to egress and inhibits liquids from exiting. In some embodiments, the MVTR of the barrier layer 210 may be greater than or equal to 300 g/m²/24 hours. In other embodiments, the MVTR of the barrier layer 210 may be greater than or equal to 1000 g/m²/24 hours. The illustrative INSPIRE 2301 film may have an MVTR (inverted cup technique) of 14400 g/m²/24 hours and may be approximately 30 microns thick. In other embodiments, a drape having a low MVTR or that allows no vapor transfer might be used. The barrier layer 210 can also function as a barrier to liquids and microorganisms.

In some embodiments, the barrier layer 210 may be adapted to form a cavity 211. For example, the barrier layer 210 may be placed on a mold and stretched to plastically deform a portion of the barrier layer 210, forming the cavity 211. A periphery of the barrier layer 210 that is not stretched by the formation of the cavity 211 may form a flange surrounding the cavity 211. In some embodiments, the cavity 211 may be positioned so that a portion of the flange may be larger on a first side of the cavity 211 than on a second side of the cavity 211. The disparity in sizes of the flange may form a foundational flange 230 and a sealing flange 231. In some embodiments, the pouch 205 may be disposed in the cavity 211. The cavity 211 may also be a portion of the barrier layer 210 that is free of the adhesive layer 213. For example, during manufacturing, a portion of the barrier layer 210 may be left without the adhesive layer 213; the area of the barrier layer 210 without the adhesive layer 213 may be equal to a surface area of the pouch 205 to be covered by the barrier layer 210.

The foundational flange 230 may extend away from the cavity 211. In some embodiments, the foundational flange 230 may have a length sufficient to permit other objects to be coupled to the dressing assembly 202. In some embodiments, the foundational flange 230 may support the negative-pressure source 204, as illustrated in FIG. 4.

The adhesive layer 213 may be coupled to the barrier layer 210 on a side of the barrier layer 210 having an opening of the cavity 211. In some embodiments, the adhesive layer 213 may include an aperture 216. The aperture 216 may be coextensive with the opening of the cavity 211. For example, the adhesive layer 213 may cover the barrier layer 210 at the foundational flange 230 and the sealing flange 231, leaving the portion of the barrier layer 210 forming the cavity 211 free of the adhesive layer 213.

In some embodiments, the bonding adhesive 212 may be deposited onto the barrier layer 210 in a pattern. For example, the bonding adhesive 212 may be applied to the barrier layer 210 on a side of the barrier layer 210 having the opening of the cavity 211 so that the bonding adhesive 212 forms a checkerboard pattern. The barrier layer 210 may have portions having the bonding adhesive 212 deposited thereon and portions that may be free of the bonding adhesive 212.

The sealing adhesive 214 may also be deposited onto the barrier layer 210 in a pattern. For example, the sealing adhesive 214 may be applied to the barrier layer 210 on the side of the barrier layer 210 having the opening of the cavity 211 so that the sealing adhesive 214 forms a checkerboard pattern. The barrier layer 210 may have portions having the sealing adhesive 214 deposited thereon and portions that may be free of the sealing adhesive 214.

The pattern of the bonding adhesive 212 and the pattern of the sealing adhesive 214 may be registered. Registration of the bonding adhesive 212 and the sealing adhesive 214 generally refers to the alignment of the two adhesives relative to one another. In particular, registration of the bonding adhesive 212 and the sealing adhesive 214 may refer to the coordination of adhesive placement on the barrier layer 210 to achieve a desired effect. For example, a certain percentage of overlap of one adhesive over the other adhesive, minimal overlap of one adhesive over the other adhesive so that the adhesives are offset from one another, or complete overlap of one adhesive over the other adhesive are all adhesive placements that may be considered registered. For example, the bonding adhesive 212 and the sealing adhesive 214 may be registered by being disposed on the barrier layer 210 so that the bonding adhesive 212 and the sealing adhesive 214 each substantially couple to the barrier layer 210. In addition, the bonding adhesive 212 and the sealing adhesive 214 of the example may be aligned relative to one another to have minimal overlap of one adhesive over the other adhesive. In another example, the sealing adhesive 214 may be offset from the bonding adhesive 212, with both adhesives being coupled to the barrier layer 210. Registering the bonding adhesive 212 and the sealing adhesive 214 provides for easier manufacturing and use of the cover 203. Registering of the bonding adhesive 212 and the sealing adhesive 214 may also enhance desired properties of the cover 203.

The bonding adhesive 212 may comprise an acrylic adhesive, rubber adhesive, high-tack silicone adhesive, polyurethane, or other substance. In an illustrative example, the bonding adhesive 212 comprises an acrylic adhesive with coating weight of 15 grams/m² (gsm) to 70 grams/m² (gsm). In some embodiments, the bond strength of the bonding adhesive may have a peel adhesion or resistance to being peeled from a stainless steel material between about 6N/25 mm to about 10N/25 mm on stainless steel substrate at 23° C. at 50% relative humidity based on the American Society for Testing and Materials (“ASTM”) standard ASTM D3330. The bonding adhesive 212 may be about 30 microns to about 60 microns in thickness.

The sealing adhesive 214 may comprise a silicone gel (or soft silicone), hydrocolloid, hydrogel, polyurethane gel, polyolefin gel, hydrogenated styrenic copolymer gels, or foamed gels with compositions as listed, or soft closed cell foams (polyurethanes, polyolefins) coated with an adhesive (e.g., 30 gsm-70 gsm acrylic), polyurethane, polyolefin, or hydrogenated styrenic copolymers. The sealing adhesive 214 may have a thickness in the range of about 100 microns (m) to about 1000 microns (m). In some embodiments, the sealing adhesive 214 may have stiffness between about 5 Shore 00 and about 80 Shore OO. The sealing adhesive 214 may be hydrophobic or hydrophilic. The sealing adhesive 214 may be an adhesive having a low to medium tackiness, for example, a silicone polymer, polyurethane, or an additional acrylic adhesive. In some embodiments, the bond strength of the sealing adhesive may have a peel adhesion or resistance to being peeled from a stainless steel material between about 0.5N/25 mm to about 1.5N/25 mm on stainless steel substrate at 23° C. at 50% relative humidity based on ASTM D3330. The sealing adhesive 214 may have a tackiness such that the sealing adhesive 214 may achieve the bond strength above after a contact time of less than 60 seconds. Tackiness may be considered a bond strength of an adhesive after a very low contact time between the adhesive and a substrate. In some embodiments, the sealing adhesive 214 may have a tackiness that may be about 30% to about 50% of the tackiness of the bonding adhesive of the bonding adhesive 212.

In the assembled state, the adhesive layer 213 may be coupled to the sealing flange 231 and the foundational flange 230. In some embodiments, the thickness of the bonding adhesive 212 may be less than the thickness of the sealing adhesive 214 so that the adhesive layer 213 may have a varying thickness. If the adhesive layer 213 is placed proximate to or in contact with the epidermis of the patient, the sealing adhesive 214 may be in contact with the epidermis to form sealing couplings. In some embodiments, the thickness of the bonding adhesive 212 may be less than the thickness of the sealing adhesive 214, forming a gap between the bonding adhesive 212 and the epidermis.

The initial tackiness of the sealing adhesive 214 is preferably sufficient to initially couple the sealing adhesive 214 to the epidermis by forming sealing couplings. Once in the desired location, a force can be applied to the barrier layer 210 of the cover 203. For example, the user may rub the foundational flange 230 and the sealing flange 231. This action can cause at least a portion of the bonding adhesive 212 to be forced into the plurality of apertures 218 and into contact with the epidermis to form bonding couplings. The bonding couplings provide secure, releasable mechanical fixation to the epidermis.

As illustrated in FIG. 4, the negative-pressure source 204, which may also be referred to as a blister, may be coupled to the barrier layer 210 of the foundational flange 230. The negative-pressure source 204 may be an enclosure formed by a film layer 232 and having a foam block 234 disposed therein. In some embodiments, the film layer 232 may form a source flange 236 and a source cavity 238. The source cavity 238 may be a portion of the film layer 232 this is plastically stretched, such as by vacuum forming, thermoforming, micro-thermoforming, injection molding, or blow molding, for example. In some embodiments, the source cavity 238 may form walls of the negative-pressure source 204 that may be resilient or flexible. The source flange 236 may be a portion of the film layer 232 adjacent to and surrounding an opening of the source cavity 238. In some embodiments, the foam block 234 may be disposed in the source cavity 238. The source flange 236 may be coupled to the barrier layer 210 of the foundational flange 230 to seal the foam block 234 in the source cavity 238. In some embodiments, the source flange 236 may be coupled to the barrier layer 210 by high frequency welding, ultrasonic welding, heat welding, or impulse welding, for example. In other exemplary embodiments, the source flange 236 may be coupled to the barrier layer 210 by bonding or folding, for example. In some embodiments, if the source flange 236 is coupled to the barrier layer 210 of the foundational flange 230, the source cavity 238 may be fluidly isolated from the ambient environment and the pouch 205.

The film layer 232 may be constructed from a material that can provide a fluid seal between two components or two environments, such as between the source cavity 238 and a local external environment, while allowing for repeated elastic deformation of the film layer 232. The film layer 232 may be, for example, an elastomeric film or membrane that can provide a seal between the source cavity 238 and the ambient environment. In some example embodiments, the film layer 232 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. In an exemplary embodiment, the film layer 232 may be a polyurethane having a thickness between about 50 microns and about 250 microns and preferably about 100 microns.

The foam block 234 may be a foam having a plurality of interconnected flow channels. For example, cellular foam, open-cell foam, reticulated foam, porous tissue collections, and other porous material that generally include pores, edges, and/or walls adapted to form interconnected fluid pathways. Liquids, gels, and other foams may also include or be cured to include apertures and flow channels. In some illustrative embodiments, the foam block 234 may be a porous foam material having interconnected cells or pores adapted to uniformly (or quasi-uniformly) distribute fluid throughout the foam block 234. The foam material may be either hydrophobic or hydrophilic. In one non-limiting example, the foam block 234 may be an open-cell, reticulated polyurethane foam such as V.A.C.® GRANUFOAM™ dressing available from Kinetic Concepts, Inc. of San Antonio, Tex. Another exemplary embodiment of the foam block 234 may be Z48AA foam from FXI®.

Foam materials may have an elastic modulus, which may also be referred to as a foam modulus. Generally, the elastic modulus of a material may measure the resistance of the material to elastic deformation under a load. The elastic modulus of a material may be defined as the slope of a stress-strain curve in the elastic deformation region of the curve. The elastic deformation region of a stress-strain curve represents that portion of the curve where a deformation of a material due to an applied load is elastic, that is, not permanent. If the load is removed, the material may return to its preloaded state. Stiffer materials may have a higher elastic modulus, and more compliant materials may have a lower elastic modulus. Generally, references to the elastic modulus of a material refers to a material under tension.

For some materials under compression, the elastic modulus can be compared between materials by comparing the compression force deflection (CFD) of the materials. Typically, CFD is determined experimentally by compressing a sample of a material until the sample is reduced to about 25% of its uncompressed size. The load applied to reach the 25% compression of the sample is then divided by the area of the sample over which the load is applied to arrive at the CFD. The CFD can also be measured by compressing a sample of a material to about 50% of the sample's uncompressed size. The CFD of a foam material can be a function of compression level, polymer stiffness, cell structure, foam density, and cell pore size. The foam block 234 may have a CFD of about 4 kPA. The foam block 234 may compress to about 25% of its uncompressed size if a load of about 4 kPa is applied to the foam block 234.

Furthermore, CFD can represent the tendency of a foam to return to its uncompressed state if a load is applied to compress the foam. For example, a foam having a CFD of about 4 kPa may exert about 4 kPa in reaction to 25% compression. The CFD of the foam block 234 may represent the ability of the foam block 234 to bias the film layer 232 toward an expanded position. For example, if the foam block 234 is compressed to 25% of its original size, the foam block 234 may exert a spring force that opposes the applied force over the area of the foam block 234 to which the force is applied. The reactive force may be proportional to the amount the foam block 234 is compressed.

The foam block 234 may have a free volume. The free volume of the foam block 234 may be the volume of free space of the foam block 234, for example, the volume of the plurality of channels of the foam block 234. In some embodiments, the free volume of the foam block 234 may be greater than the free volume of the sealed therapeutic environment. For example, the free volume of the foam block 234 may be greater than the free volume of the pouch 205. If the free volume of the pouch 205 is about 10 cm³, then the free volume of the foam block 234 may be greater than about 10 cm³.

In some embodiments, the negative-pressure source 204 may be fluidly coupled to the cavity 211 through a fluid inlet, such as a tube 240. The tube 240 may be representative of a fluid communication path between the negative-pressure source 204 and the cavity 211. In other embodiments, the tube 240 may be a sealed channel or other fluid pathway. The tube 240 may include a lumen 242 fluidly coupled to the source cavity 238 and the pouch 205. In some embodiments, a valve, such as a check valve 244, may be fluidly coupled to the lumen 242. Exemplary check valves 244 may include ball check valves, diaphragm check valves, swing check valves, stop-check valves, duckbill valves, or pneumatic non-return valves. The check valve 244 may permit fluid communication from the pouch 205 to the source cavity 238 and prevent fluid communication from the source cavity 238 to the pouch 205. For example, if a pressure in the pouch 205 is greater than a pressure in the source cavity 238, the check valve 244 may open, and if the pressure in the source cavity 238 is greater than the pressure in the pouch 205, the check valve 244 may close. In some embodiments, a filter may be disposed on an end of the tube 240. The filter may be a hydrophobic porous polymer filter having gel blocking properties.

The source cavity 238 may also be fluidly coupled to the ambient environment through a fluid outlet, such as a tube 246. The tube 246 may be representative of a fluid communication path between the ambient environment and the source cavity 238. For example, the tube 246 having a lumen 248 may fluidly couple the source cavity 238 to the ambient environment. A valve, such as a check valve 250, may be fluidly coupled to the lumen 248 to control fluid communication through the lumen 248. Exemplary check valves 250 may include ball check valves, diaphragm check valves, swing check valves, stop-check valves, duckbill valves, or pneumatic non-return valves. In some embodiments, the check valve 250 may permit fluid communication from the source cavity 238 to the ambient environment and prevent fluid communication from the ambient environment to the source cavity 238. For example, if a pressure in the source cavity 238 is greater than a pressure in the ambient environment, the check valve 250 may open, and if the pressure in the ambient environment is greater than the pressure in the source cavity 238, the check valve 250 may close. In some embodiments, a filter may be disposed on an end of the tube 246. The filter may be a hydrophobic porous polymer filter having gel blocking properties.

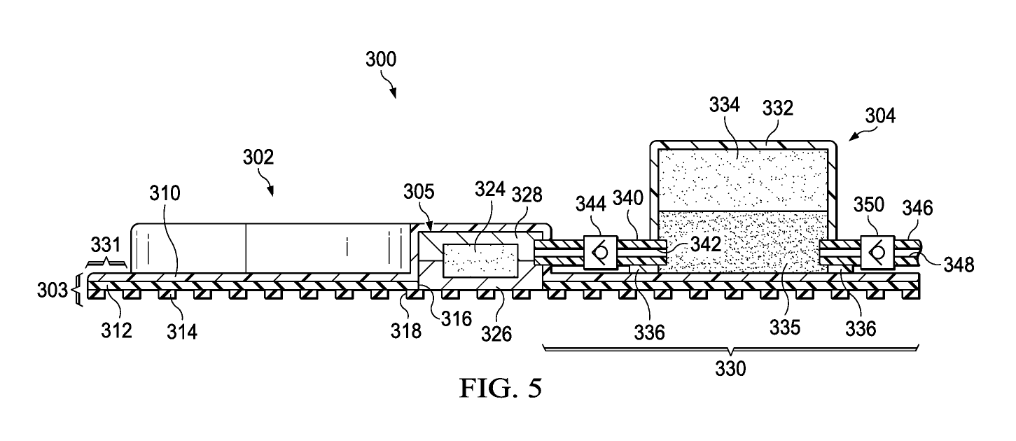

FIG. 5 is a sectional view of an example embodiment of a negative-pressure therapy system 300 that can provide negative-pressure therapy in accordance with this specification. The negative-pressure therapy system 300 may be similar to and operate as described above with respect to the negative-pressure therapy system 100. Similar elements have similar reference numbers indexed to 300. As shown in FIG. 5, the negative-pressure therapy system 300 can include a dressing assembly 302 having a cover 303, a pouch 305, and a negative-pressure source 304. The cover 303, the pouch 305, and the negative-pressure source 304 may be coupled to each other. In some embodiments, the negative-pressure therapy system 300 can also include the tissue interface 108.

The pouch 305 may include an absorbent 324, a first outer layer, such as an upstream layer 326, and a second outer layer, such as a downstream layer 328. The upstream layer 326 and the downstream layer 328 may envelop or enclose the absorbent 324. The absorbent 324 may hold, stabilize, and/or solidify fluids that may be collected from the tissue site. The absorbent 324 may be formed from materials referred to as “hydrogels,” “super-absorbents,” or “hydrocolloids.” If disposed within the dressing assembly 302, the absorbent 324 may be formed into fibers or spheres to manifold negative pressure until the absorbent 324 becomes saturated. Spaces or voids between the fibers or spheres may allow a negative pressure that is supplied to the dressing assembly 302 to be transferred within and through the absorbent 324 to the tissue site. In some exemplary embodiments, the absorbent 324 may be Texsus FP2325 having a material density of about 800 grams per square meter (gsm). In other exemplary embodiments, the absorbent material may be BASF 402C, Technical Absorbents 2317 available from Technical Absorbents (www.techabsorbents.com), sodium polyacrylate super absorbers, cellulosics (carboxy methyl cellulose and salts such as sodium CMC), or alginates.

In some exemplary embodiments, the absorbent 324 may be formed of granular absorbent components that may be scatter coated onto a paper substrate. Scatter coating involves spreading a granular absorbent powder uniformly onto a textile substrate, such as paper. The substrate, having the granular absorbent powder disposed thereon, may be passed through an oven to cure the powder and cause the powder to adhere to the paper substrate. The cured granular absorbent powder and substrate may be passed through a calender machine to provide a smooth uniform surface to the absorbent material.

In some exemplary embodiments, the upstream layer 326 and the downstream layer 328 have perimeter dimensions that may be larger than the perimeter dimensions of the absorbent 324 so that, if the absorbent 324 is positioned between the upstream layer 326 and the downstream layer 328 and the center portions of the absorbent 324, the upstream layer 326, and the downstream layer 328 are aligned, the upstream layer 326 and the downstream layer 328 may extend beyond the perimeter of the absorbent 324. In some exemplary embodiments, the upstream layer 326 and the downstream layer 328 surround the absorbent 324. Peripheral portions of the upstream layer 326 and the downstream layer 328 may be coupled so that the upstream layer 326 and the downstream layer 328 enclose the absorbent 324. The upstream layer 326 and the downstream layer 328 may be coupled by high frequency welding, ultrasonic welding, heat welding, or impulse welding, for example. In other exemplary embodiments, the upstream layer 326 and the downstream layer 328 may be coupled by bonding or folding, for example.

The upstream layer 326 may be formed of non-woven material in some embodiments. For example, the upstream layer 326 may have a polyester fibrous porous structure. The upstream layer 326 may be porous, but preferably the upstream layer 326 is not perforated. The upstream layer 326 may have a material density between about 80 gsm and about 150 gsm. In other exemplary embodiments, the material density may be lower or greater depending on the particular application of the pouch 305. In some embodiments, the upstream layer 326 may a plurality of layers of, for example, non-woven material. The upstream layer 326 may be formed of Libeltex TDL2, for example. In other embodiments, the upstream layer 326 may also be formed of Libeltex TL4. The upstream layer 326 may have a hydrophilic side and a hydrophobic side.

The downstream layer 328 may also be formed of a non-woven material in some embodiments. For example, the downstream layer 328 may have a polyester fibrous porous structure. The downstream layer 328 may be porous, but the downstream layer 328 preferably is not perforated. The downstream layer 328 may have a material density between about 80 gsm and about 150 gsm. In other exemplary embodiments, the material density may be lower or greater depending on the particular application of the pouch 305. The material density of the downstream layer 328 may be greater or less than the material density of the upstream layer 326. In some embodiments, a thickness of the downstream layer 328 may be greater than a thickness of the upstream layer 326. In other embodiments, the thickness of the downstream layer 328 may be less than the thickness of the upstream layer 326. In some embodiments, the downstream layer 328 may a plurality of layers of, for example, non-woven material. The downstream layer 328 may be formed of Libeltex TL4. In other exemplary embodiments, the downstream layer 328 may be formed of Libeltex TDL2.

The upstream layer 326 and the downstream layer 328 may be manifolding layers configured to facilitate fluid movement through the pouch 305. In some embodiments, the upstream layer 326 and the downstream layer 328 may each have a hydrophobic side and a hydrophilic side. The hydrophobic side may also be referred to as a wicking side, wicking surface, distribution surface, distribution side, or fluid distribution surface. The hydrophobic side may be a smooth distribution surface configured to move fluid along a grain of the upstream layer 326 and the downstream layer 328, distributing fluid throughout the upstream layer 326 and the downstream layer 328. The hydrophilic side may be configured to acquire bodily fluid from the hydrophobic side to aid in bodily fluid movement into the absorbent 324. The hydrophilic side may also be referred to as a fluid acquisition surface, fluid acquisition side, hydrophilic acquisition surface, or hydrophilic acquisition side. The hydrophilic side may be a fibrous surface and be configured to draw fluid into the upstream layer 326 and the downstream layer 328. In some embodiments, the hydrophilic side of the upstream layer 326 and the downstream layer 328 may be positioned adjacent to the absorbent 324. In other embodiments, the hydrophobic side of the upstream layer 326 and the downstream layer 328 may be positioned adjacent to the absorbent 324. In still other embodiments, the hydrophilic side of one of the upstream layer 326 or the downstream layer 328 may be positioned adjacent to the absorbent 324, and the hydrophobic side of the other of the upstream layer 326 or the downstream layer 328 may be positioned adjacent to the absorbent 324.

In some embodiments, the cover 303 may include or may be a hybrid drape that includes a barrier layer 310, a bonding adhesive layer 312, and a sealing adhesive layer 314. The barrier layer 310 may be formed from a range of medically approved films ranging in thickness from about 15 microns (m) to about 50 microns (m). The barrier layer 310 may comprise a suitable material or materials, such as the following: hydrophilic polyurethane (PU), cellulosics, hydrophilic polyamides, polyvinyl alcohol, polyvinyl pyrrolidone, hydrophilic acrylics, hydrophilic silicone elastomers, and copolymers of these. In some embodiments, the barrier layer 310 may be formed from a breathable cast matt polyurethane film sold by Transcontinental Advanced Coatings of Wrexham, United Kingdom, under the name INSPIRE 2301.

The barrier layer 310 may have a high moisture vapor transmission rate (MVTR). The MVTR of the barrier layer 310 allows vapor to egress and inhibits liquids from exiting. In some embodiments, the MVTR of the barrier layer 310 may be greater than or equal to 300 g/m²/24 hours. In other embodiments, the MVTR of the barrier layer 310 may be greater than or equal to 1000 g/m²/24 hours. The illustrative INSPIRE 2301 film may have an MVTR (inverted cup technique) of 14400 g/m²/24 hours and may be approximately 30 microns thick. In other embodiments, a drape having a low MVTR or that allows no vapor transfer might be used. The barrier layer 310 can also function as a barrier to liquids and microorganisms.

In some embodiments, the barrier layer 310 may be adapted to form a bulge on a first side of the barrier layer 310 and a cavity 311 on an opposite side of the barrier layer 310. For example, the barrier layer 310 may be placed on a mold and stretched to plastically deform a portion of the barrier layer 310, forming the cavity 311. A periphery of the barrier layer 310 that is not stretched by the formation of the cavity 311 may form a flange surrounding the cavity 311. In some embodiments, the cavity 311 may be positioned so that a portion of the flange may be larger on a first side of the cavity 311 than on a second side of the cavity 311. The disparity in sizes of the flange may form a foundational flange 330 and a sealing flange 331. In some embodiments, the pouch 305 may be disposed in the cavity 311. The cavity 311 may also be a portion of the barrier layer 310 that is free of the bonding adhesive layer 312. For example, during manufacturing, a portion of the barrier layer 310 may be left without the bonding adhesive layer 312; the area of the barrier layer 310 without the bonding adhesive layer 312 may be equal to a surface area of the pouch 305 to be covered by the barrier layer 310.

The foundational flange 330 may extend away from the cavity 311. In some embodiments, the foundational flange 330 may have a length and a width sufficient to permit other objects to be coupled to the dressing assembly 302. In some embodiments, the foundational flange 330 may support the negative-pressure source 304, as illustrated in FIG. 5.

The bonding adhesive layer 312 may be coupled to the barrier layer 310 on a side of the barrier layer 310 having an opening of the cavity 311. In some embodiments, the bonding adhesive layer 312 may include an aperture 316. The aperture 316 may be coextensive with the opening of the cavity 311. For example, the bonding adhesive layer 312 may cover the barrier layer 310 at the foundational flange 330 and the sealing flange 331, leaving the portion of the barrier layer 310 forming the cavity 311 free of bonding adhesive.

The bonding adhesive layer 312 may comprise an acrylic adhesive, rubber adhesive, high-tack silicone adhesive, polyurethane, or other substance. In an illustrative example, the bonding adhesive layer 312 comprises an acrylic adhesive with coating weight of 15 grams/m² (gsm) to 70 grams/m² (gsm). The bonding adhesive layer 312 may be a continuous layer of material or may be a layer with apertures (not shown). The apertures may be formed after application of the bonding adhesive layer 312 or may be formed by coating the bonding adhesive layer 312 in patterns on a carrier layer. In some embodiments, the bond strength of the bonding adhesive may have a peel adhesion or resistance to being peeled from a stainless steel material between about 6N/25 mm to about 10N/25 mm on stainless steel substrate at 23° C. at 50% relative humidity based on the American Society for Testing and Materials (“ASTM”) standard ASTM D3330. The bonding adhesive layer 312 may be about 30 microns to about 60 microns in thickness.

The sealing adhesive layer 314 may be coupled to the bonding adhesive layer 312 and the pouch 305. For example, the sealing adhesive layer 314 may cover the sealing flange 331, the pouch 305, and the foundational flange 330. The sealing adhesive layer 314 may be formed with the plurality of apertures 318. The apertures 318 may be numerous shapes, for example, circles, squares, stars, ovals, polygons, slits, complex curves, rectilinear shapes, triangles, or other shapes. Each aperture 318 of the plurality of apertures 318 may have an effective diameter, which is the diameter of a circular area having the same surface area as the aperture 318. The average effective diameter of each aperture 318 may typically be in the range of about 6 mm to about 50 mm. The plurality of apertures 318 may have a uniform pattern or may be randomly distributed in the sealing adhesive layer 314. Generally, the apertures 318 may be disposed across a length and width of the sealing adhesive layer 314.

The sealing adhesive layer 314 may comprise a silicone gel (or soft silicone), hydrocolloid, hydrogel, polyurethane gel, polyolefin gel, hydrogenated styrenic copolymer gels, or foamed gels with compositions as listed, or soft closed cell foams (polyurethanes, polyolefins) coated with an adhesive (e.g., 30 gsm-70 gsm acrylic), polyurethane, polyolefin, or hydrogenated styrenic copolymers. The sealing adhesive layer 314 may have a thickness in the range of about 100 microns (μm) to about 1000 microns (μm). In some embodiments, the sealing adhesive layer 314 may have stiffness between about 5 Shore 00 and about 80 Shore OO. The sealing adhesive layer 314 may be hydrophobic or hydrophilic. The sealing adhesive of the sealing adhesive layer 314 may be an adhesive having a low to medium tackiness, for example, a silicone polymer, polyurethane, or an additional acrylic adhesive. In some embodiments, the bond strength of the sealing adhesive may have a peel adhesion or resistance to being peeled from a stainless steel material between about 0.5N/25 mm to about 1.5N/25 mm on stainless steel substrate at 23° C. at 50% relative humidity based on ASTM D3330. The sealing adhesive of the sealing adhesive layer 314 may have a tackiness such that the sealing adhesive may achieve the bond strength above after a contact time of less than 60 seconds. Tackiness may be considered a bond strength of an adhesive after a very low contact time between the adhesive and a substrate. In some embodiments, the sealing adhesive layer 314 may have a tackiness that may be about 30% to about 50% of the tackiness of the bonding adhesive of the bonding adhesive layer 312.

In the assembled state, the bonding adhesive layer 312 may be coupled to the barrier layer 310. The sealing adhesive layer 314 may be coupled to the bonding adhesive layer 312 at the sealing flange 331 and the foundational flange 330 and to the pouch 305 at the cavity 311. In some embodiments, a scrim layer may be disposed in the sealing adhesive layer 314. The scrim layer may provide additional mechanical support for the sealing adhesive layer 314. In some embodiments, the sealing adhesive layer 314 may be treated on a portion and a side of the sealing adhesive layer 314 adjacent to the pouch 305. The treated portion of the sealing adhesive layer 314 may reduce the tackiness of the sealing adhesive layer 314 so that the sealing adhesive layer 314 may not readily adhere to the pouch 305. The initial tackiness of the sealing adhesive layer 314 is preferably sufficient to initially couple the sealing adhesive layer 314 to the epidermis by forming sealing couplings. Once in the desired location, a force can be applied to the barrier layer 310 of the cover 303. For example, the user may rub the foundational flange 330 and the sealing flange 331. This action can cause at least a portion of the bonding adhesive layer 312 to be forced into the plurality of apertures 318 and into contact with the epidermis to form bonding couplings. The bonding couplings provide secure, releasable mechanical fixation to the epidermis.

The average effective diameter of the plurality of apertures 318 for the sealing adhesive layer 314 may be varied as one control of the tackiness or adhesion strength of the cover 303. In this regard, there is interplay between three main variables for each embodiment: the thickness of the sealing adhesive layer 314, the average effective diameter of the plurality of apertures 318, and the tackiness of the bonding adhesive layer 312. The more bonding adhesive of the bonding adhesive layer 312 that extends through the apertures 318, the stronger the bond of the bonding coupling. The thinner the sealing adhesive layer 314, the more bonding adhesive of the bonding adhesive layer 312 generally extends through the apertures 318 and the greater the bond of the bonding coupling. As an example of the interplay, if a very tacky bonding adhesive layer 312 is used and the thickness of the sealing adhesive layer 314 is small, the average effective diameter of the plurality of apertures 318 may be relatively smaller than apertures 318 in a thicker sealing adhesive layer 314 and less tacky bonding adhesive layer 312. In some embodiments, the thickness of the sealing adhesive layer 314 may be approximately 200 microns, the thickness of the bonding adhesive layer 312 is approximately 30 microns with a tackiness of 2000 g/25 cm wide strip, and the average effective diameter of each aperture 318 is approximately about 6 mm.

As illustrated in FIG. 5, the negative-pressure source 304, which may also be referred to as a blister, may be coupled to the barrier layer 310 of the foundational flange 330. The negative-pressure source 304 may include a barrier layer and a biasing member, for example, a film layer 332, a first foam block 334, and a second foam block 335. In some embodiments, the film layer 332 may form a source flange 336 and a source cavity 338. The source cavity 338 may be a portion of the film layer 332 that is plastically deformed, such as by vacuum forming, thermoforming, micro-thermoforming, injection molding, or blow molding, for example. In some embodiments, the source cavity 338 may form walls of the negative-pressure source 304 that may be resilient or flexible. The source flange 336 may be a portion of the film layer 332 adjacent to and surrounding an opening of the source cavity 338. In some embodiments, the first foam block 334 and the second foam block 335 may be disposed in the source cavity 338. For example, the first foam block 334 and the second foam block 335 may be stacked over one another and positioned within the source cavity 338. The source flange 336 may be coupled to the barrier layer 310 of the foundational flange 330 to seal the first foam block 334 and the second foam block 335 in the source cavity 338. In some embodiments, the source flange 336 may be coupled to the barrier layer 310 by high frequency welding, ultrasonic welding, heat welding, or impulse welding, for example. In other exemplary embodiments, the source flange 336 may be coupled to the barrier layer 310 by bonding or folding, for example. In some embodiments, if the source flange 336 is coupled to the barrier layer 310 of the foundational flange 330, the source cavity 338 may be fluidly isolated from the ambient environment and the pouch 305.

The film layer 332 may be constructed from a material that can provide a fluid seal between two components or two environments, such as between the source cavity 238 and a local external environment, while allowing for repeated elastic deformation of the film layer 332. The film layer 332 may be, for example, an elastomeric film or membrane that can provide a seal between the source cavity 338 and the ambient environment. In some example embodiments, the film layer 332 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. In an exemplary embodiment, the film layer 332 may be a polyurethane having a thickness between about 50 microns and about 250 microns and preferably about 100 microns.

The first foam block 334 and the second foam block 335 may have similar dimensions. For example, if the first foam block 334 and the second foam block 335 are cylindrical, the first foam block 334 and the second foam block 335 may have similar diameters. The first foam block 334 and the second foam block 335 may be a foam having a plurality of interconnected flow channels. For example, cellular foam, open-cell foam, reticulated foam, porous tissue collections, and other porous material that generally include pores, edges, and/or walls adapted to form interconnected fluid pathways. Liquids, gels, and other foams may also include or be cured to include apertures and flow channels. In some illustrative embodiments, the first foam block 334 and the second foam block 335 may be a porous foam material having interconnected cells or pores adapted to uniformly (or quasi-uniformly) distribute fluid throughout the first foam block 334 and the second foam block 335. The foam material may be either hydrophobic or hydrophilic. In one non-limiting example, the first foam block 334 and the second foam block 335 may be an open-cell, reticulated polyurethane foam such as V.A.C.® GRANUFOAM™ dressing available from Kinetic Concepts, Inc. of San Antonio, Tex. Another exemplary embodiment of the first foam block 334 and the second foam block 335 may be Z48AA foam from FXI.

Foam materials may have an elastic modulus, which may also be referred to as a foam modulus. Generally, the elastic modulus of a material may measure the resistance of the material to elastic deformation under a load. The elastic modulus of a material may be defined as the slope of a stress-strain curve in the elastic deformation region of the curve. The elastic deformation region of a stress-strain curve represents that portion of the curve where the deformation of the material due to the applied load is elastic, that is, not permanent. If the load is removed, the material may return to its preloaded state. Stiffer materials may have a higher elastic modulus, and more compliant materials may have a lower elastic modulus. Generally, references to the elastic modulus of a material refers to a material under tension.

For foam materials under compression, the elastic modulus can compared between materials by comparing the compression force deflection (CFD) of the materials. Typically, CFD is determined experimentally by compressing a sample of a material until the sample is reduced to about 25% of its uncompressed size. The load applied to reach the 25% compression of the sample is then divided by the area of the sample over which the load is applied to arrive at the CFD. The CFD can also be measured by compressing a sample of a material to about 50% of the sample's uncompressed size. The CFD of a foam material can be a function of compression level, polymer stiffness, cell structure, foam density, and cell pore size. In some embodiments, the first foam block 334 and the second foam block 335 may have a CFD that is greater than a CFD of the tissue interface 108. For example, the tissue interface 108 may have a 25% CFD of about 2 kPa. The tissue interface 108 may compress to about 25% of its uncompressed size if a load of about 2 kPa is applied to the tissue interface 108. The first foam block 334 and the second foam block 335 may have a CFD of about 4 kPA. The first foam block 334 and the second foam block 335 may compress to about 25% of its uncompressed size if a load of about 4 kPa is applied to the first foam block 334 and the second foam block 335. Thus, the first foam block 334 and the second foam block 335 is more resistant to deformation than the tissue interface 108.

Furthermore, CFD can represent the tendency of a foam to return to its uncompressed state if a load is applied to compress the foam. For example, a foam having a CFD of about 4 kPa may exert about 4 kPa in reaction to 25% compression. The CFD of the first foam block 334 and the second foam block 335 may represent the ability of the first foam block 334 and the second foam block 335 to bias the film layer 332 toward an expanded position. For example, if the first foam block 334 and the second foam block 335 is compressed to 25% of its original size, the first foam block 334 and the second foam block 335 may exert a spring force that opposes the applied force over the area of the first foam block 334 and the second foam block 335 to which the force is applied. The reactive force may be proportional to the amount the first foam block 334 and the second foam block 335 is compressed.

In some embodiments, the first foam block 334 and the second foam block 335 may have different foam moduli. For example, the first foam block 334 may have a first CFD so that the first foam block 334 may exert a first force when in the compressed state that decreases as the first foam block 334 extends to the uncompressed state. Similarly, the second foam block 335 may have a second CFD so that the second foam block 335 may exert a second force when in the compressed state that decreases as the second foam block 335 extends to the uncompressed state. If the first foam block 334 and the second foam block 335 are stacked, the first force and the second force may be combined to reach a total desired spring force. In some embodiments, the CFD of the first foam block 334 and the CFD of the second foam block 335 may be selected so that the total desired spring force for the combined first foam block 334 and the second foam block 335 is generally the same approaching the extended state as in the compressed state. For example, the first foam block 334 and the second foam block 335 may be selected so that the collective blocks exert the same upward force over the entire extension of both the first foam block 334 and the second foam block 335.

The foam material of the first foam block 334 and the second foam block 335 may be selected based on an expected volume of the pouch 305 and the tissue interface 108 (if used). The volume of the pouch 305 may define a volume of fluid to be withdrawn from the pouch 305 to achieve a therapy pressure. For example, if the pouch 305 has a volume of about 50 cubic centimeters, and no tissue interface 108 is used, removing about 10 cubic centimeters of fluid from the pouch 305 may generate a negative pressure of about 125 mm Hg. To generate 125 mm Hg with a single compression of a single foam block having a volume of 10 cm³ the CFD of the single foam block may be around 17 kPa. Similarly, the moduli of the first foam block 334 and the second foam block 335 may be selected to have a combined foam modulus of about 17 kPa. Having the first foam block 334 and the second foam block 335 may allow for selection of two foams having lower than 17 kPa moduli, which may each be more easily compressed than a single foam having the 17 kPa modulus.

The first foam block 334 and the second foam block 335 may have a free volume. The free volume of first foam block 334 and the second foam block 335 may be the volume of free space of the first foam block 334 and the second foam block 335, for example, the volume of the plurality of channels of the first foam block 334 and the second foam block 335. In some embodiments, the free volume of the first foam block 334 and the second foam block 335 may be greater than the free volume of the pouch 305. For example, if the free volume of the pouch 305 is 10 cm³, then the free volume of the first foam block 334 and the second foam block 335 may be greater than about 20 cm³.

In some embodiments, the negative-pressure source 304 may be fluidly coupled to the cavity 311 through a fluid inlet, such as a tube 340. The tube 340 may be representative of a fluid communication path between the negative-pressure source 304 and the cavity 311. In other embodiments, the tube 340 may be a sealed channel or other fluid pathway. The tube 340 may include a lumen 342 fluidly coupled to the source cavity 338 and the pouch 305. In some embodiments, a valve, such as a check valve 344, may be fluidly coupled to the lumen 342. Exemplary check valves 344 may include ball check valves, diaphragm check valves, swing check valves, stop-check valves, duckbill valves, or pneumatic non-return valves. The check valve 344 may permit fluid communication from the pouch 305 to the source cavity 338 and prevent fluid communication from the source cavity 338 to the pouch 305. For example, if a pressure in the pouch 305 is greater than a pressure in the source cavity 338, the check valve 344 may open, and if the pressure in the source cavity 338 is greater than the pressure in the pouch 305, the check valve 344 may close. In some embodiments, a filter may be disposed on an end of the tube 340. The filter may be a hydrophobic porous polymer filter having gel blocking properties.

The source cavity 338 may also be fluidly coupled to the ambient environment through a fluid outlet, such as a tube 346. For example, the tube 346 having a lumen 348 may fluidly couple the source cavity 338 to the ambient environment. The tube 346 may be representative of a fluid communication path between the ambient environment and the source cavity 338. A valve, such as a check valve 350, may be fluidly coupled to the lumen 348 to control fluid communication through the lumen 348. Exemplary check valves 350 may include ball check valves, diaphragm check valves, swing check valves, stop-check valves, duckbill valves, or pneumatic non-return valves. In some embodiments, the check valve 350 may permit fluid communication from the source cavity 338 to the ambient environment and prevent fluid communication from the ambient environment to the source cavity 338. For example, if a pressure in the source cavity 338 is greater than a pressure in the ambient environment, the check valve 350 may open, and if the pressure in the ambient environment is greater than the pressure in the source cavity 338, the check valve 350 may close. In some embodiments, a filter may be disposed on an end of the tube 346. The filter may be a hydrophobic porous polymer filter having gel blocking properties.

The dressing assembly 302 may be disposed over the tissue site to form the sealed therapeutic environment. In some embodiments, the pouch 305 of the dressing assembly 302 may be positioned over the tissue site and the negative-pressure source 304 may be positioned over undamaged tissue proximate the tissue interface 108. A force, such as hand pressure, may be applied to the sealing flange 331 and the foundational flange 330, urging the bonding adhesive of the bonding adhesive layer 312 through the apertures 318 of the sealing adhesive layer 314 to form bonding couplings and securing the negative-pressure therapy system 300 to the tissue site.

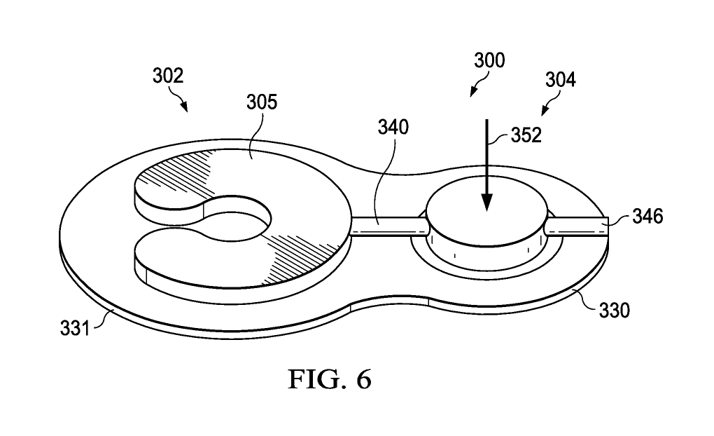

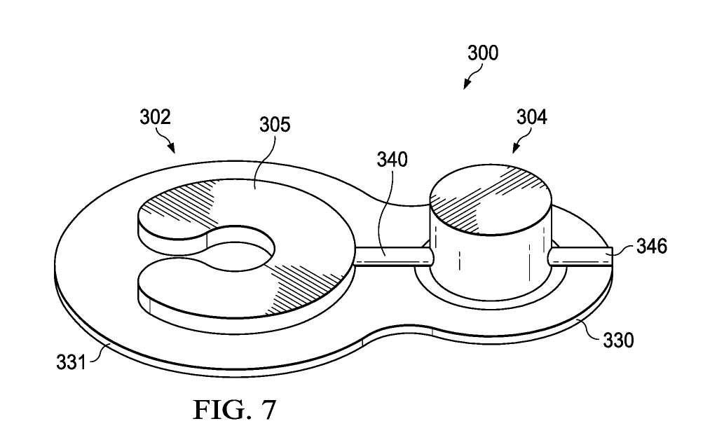

FIG. 6 is a perspective view illustrating additional details of the negative-pressure source 304 in a first position, and FIG. 7 is a perspective view illustrating additional details of the negative-pressure source 304 is a second position. Once positioned, the negative-pressure source 304 may be operated to generate a negative pressure in the pouch 305. As shown in FIG. 6, a force 352, such as hand pressure, may be applied to the film layer 332 over the first foam block 334 to compress the first foam block 334 to the first position and decrease the volume of the source cavity 338. If the first foam block 334 and the source cavity 338 are fluidly isolated from the ambient environment, compression of the first foam block 334 may increase the pressure in the source cavity 338. An increase of pressure in the source cavity 338 may create a pressure differential across the check valve 344 that urges the check valve 344 to close. Similarly, an increase of pressure in the source cavity 338 may create a pressure differential across the check valve 350 that urges the check valve 350 to open, allowing fluid from the source cavity 338 to flow through the tube 346 to the ambient environment. If the force 352 is removed, the first foam block 334 may expand, increasing the volume of the source cavity 338 and decreasing the pressure in the source cavity 338. In response, the decrease in pressure in the source cavity 338 may create a pressure differential across the check valve 350 that urges the check valve 350 to close, preventing fluid from flowing from the ambient environment to the source cavity 338. The decrease in pressure in the source cavity 338 may also create a pressure differential across the check valve 344 that urges the check valve 344 to open, permitting fluid flow from the pouch 305 to the source cavity 338. Fluid may flow from the pouch 305 to the source cavity 338 until the source cavity 338 and the first foam block 334 reach their respective uncompressed positions as shown in FIG. 7. In this manner, a portion of the total volume of fluid in the sealed therapeutic environment may be removed. In response to the removal of a portion of the fluid, a smaller volume of fluid occupies the sealed therapeutic environment, decreasing the pressure. Each time the first foam block 334 is compressed and allowed to rebound, additional fluid may be removed from the sealed therapeutic environment, further decreasing the pressure.

Decreasing the pressure in the source cavity 338, the cavity 311, and the cavity between the pouch 305 and the tissue site may create a pressure differential across the dressing assembly 302. If the pressure in the source cavity 338, the cavity 311, and the cavity between the pouch 305 and the tissue site reaches the therapy pressure for negative-pressure therapy, the CFD of the first foam block 334 may be insufficient to cause the first foam block 334 to expand following compression of the first foam block 334 from the second position of FIG. 7 to the first position of FIG. 6. The therapy pressure may be the pressure at which negative-pressure therapy may be performed. In some embodiments, the therapy pressure provided by the first foam block 334 may be about 70 mm Hg of negative pressure. In other embodiments, the therapy pressure provided by the first foam block 334 may be between about 50 mm Hg and 150 mm Hg of negative pressure. If the first foam block 334 remains compressed as shown in FIG. 6, a patient or clinician may have an indication that the therapy pressure has been reached. The compressed first foam block 334 may also act as a pressure reservoir. As negative-pressure therapy is provided, there may be a natural leakage or decline of negative pressure at the tissue site. As the negative pressure decreases in the cavity 311, the source cavity 338, and the cavity between the pouch 305 and the tissue site, the pressure differential across the dressing assembly 302 may decrease and the first foam block 334 may gradually expand, reapplying negative pressure at the tissue site. In some embodiments, the negative-pressure source 304 having the first foam block 334 may maintain a therapeutic negative pressure for about 8 hours or more.

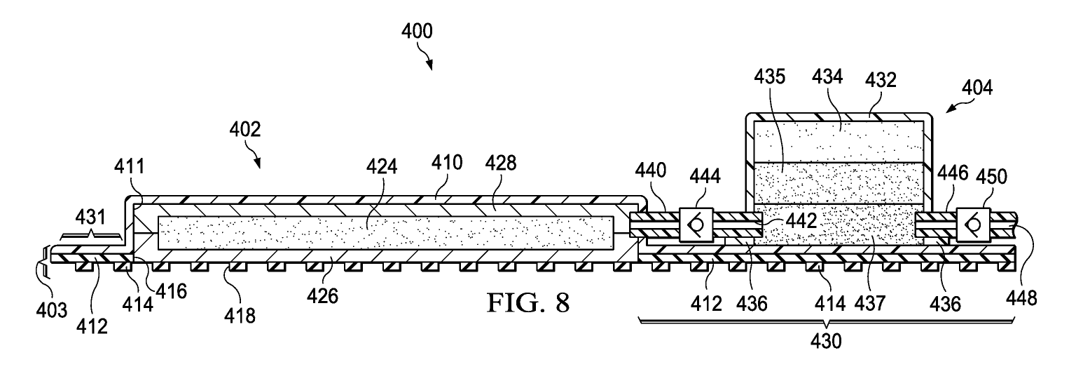

FIG. 8 is a sectional view of an example embodiment of a negative-pressure therapy system 400 that can provide negative-pressure therapy in accordance with this specification. The negative-pressure therapy system 400 may be similar to and operate as described above with respect to the negative-pressure therapy system 100. Similar elements have similar reference numbers indexed to 400. As shown in FIG. 8, the negative-pressure therapy system 400 can include a dressing assembly 402 having a cover 403, a pouch 405, and a negative-pressure source 404. The cover 403, the pouch 405, and the negative-pressure source 404 may be coupled to each other. In some embodiments, the negative-pressure therapy system 400 can also include the tissue interface 108.

The pouch 405 may include an absorbent 424, a first outer layer, such as an upstream layer 426, and a second outer layer, such as a downstream layer 428. The upstream layer 426 and the downstream layer 428 may envelop or enclose the absorbent 424. The absorbent 424 may hold, stabilize, and/or solidify fluids that may be collected from the tissue site. The absorbent 424 may be formed from materials referred to as “hydrogels,” “super-absorbents,” or “hydrocolloids.” If disposed within the dressing assembly 402, the absorbent 424 may be formed into fibers or spheres to manifold negative pressure until the absorbent 424 becomes saturated. Spaces or voids between the fibers or spheres may allow a negative pressure that is supplied to the dressing assembly 402 to be transferred within and through the absorbent 424 to the tissue site. In some exemplary embodiments, the absorbent 424 may be Texsus FP2325 having a material density of about 800 grams per square meter (gsm). In other exemplary embodiments, the absorbent material may be BASF 402C, Technical Absorbents 2317 available from Technical Absorbents (www.techabsorbents.com), sodium polyacrylate super absorbers, cellulosics (carboxy methyl cellulose and salts such as sodium CMC), or alginates.

In some exemplary embodiments, the absorbent 424 may be formed of granular absorbent components that may be scatter coated onto a paper substrate. Scatter coating involves spreading a granular absorbent powder uniformly onto a textile substrate, such as paper. The substrate, having the granular absorbent powder disposed thereon, may be passed through an oven to cure the powder and cause the powder to adhere to the paper substrate. The cured granular absorbent powder and substrate may be passed through a calender machine to provide a smooth uniform surface to the absorbent material.

In some exemplary embodiments, the upstream layer 426 and the downstream layer 428 have perimeter dimensions that may be larger than the perimeter dimensions of the absorbent 424 so that, if the absorbent 424 is positioned between the upstream layer 426 and the downstream layer 428 and the center portions of the absorbent 424, the upstream layer 426, and the downstream layer 428 are aligned, the upstream layer 426 and the downstream layer 428 may extend beyond the perimeter of the absorbent 424. In some exemplary embodiments, the upstream layer 426 and the downstream layer 428 may surround the absorbent 424. Peripheral portions of the upstream layer 426 and the downstream layer 428 may be coupled so that the upstream layer 426 and the downstream layer 428 enclose the absorbent 424. The upstream layer 426 and the downstream layer 428 may be coupled by high frequency welding, ultrasonic welding, heat welding, or impulse welding, for example. In other exemplary embodiments, the upstream layer 426 and the downstream layer 428 may be coupled by bonding or folding, for example.

The upstream layer 426 may be formed of non-woven material in some embodiments. For example, the upstream layer 426 may have a polyester fibrous porous structure. The upstream layer 426 may be porous, but preferably the upstream layer 426 is not perforated. The upstream layer 426 may have a material density between about 80 gsm and about 150 gsm. In other exemplary embodiments, the material density may be lower or greater depending on the particular application of the pouch 405. The upstream layer 426 may be formed of Libeltex TDL2, for example. In other embodiments, the upstream layer 426 may be formed of Libeltex TL4. The upstream layer 426 may have a hydrophilic side and a hydrophobic side.

The downstream layer 428 may also be formed of a non-woven material in some embodiments. For example, the downstream layer 428 may have a polyester fibrous porous structure. The downstream layer 428 may be porous, but the downstream layer 428 preferably is not perforated. The downstream layer 428 may have a material density between about 80 gsm and about 150 gsm. In other exemplary embodiments, the material density may be lower or greater depending on the particular application of the pouch 405. The material density of the downstream layer 428 may be greater or less than the material density of the upstream layer 426. In some embodiments, a thickness of the downstream layer 428 may be greater than a thickness of the upstream layer 426. In other embodiments, the thickness of the downstream layer 428 may be less than the thickness of the upstream layer 426. The downstream layer 428 may be formed of Libeltex TL4. In other exemplary embodiments, the downstream layer 428 may be formed of Libeltex TDL2.

The upstream layer 426 and the downstream layer 428 may be manifolding layers configured to facilitate fluid movement through the pouch 405. In some embodiments, the upstream layer 426 and the downstream layer 428 may each have a hydrophobic side and a hydrophilic side. The hydrophobic side may also be referred to as a wicking side, wicking surface, distribution surface, distribution side, or fluid distribution surface. The hydrophobic side may be a smooth distribution surface configured to move fluid along a grain of the upstream layer 426 and the downstream layer 428, distributing fluid throughout the upstream layer 426 and the downstream layer 428. The hydrophilic side may be configured to acquire bodily fluid from the hydrophobic side to aid in bodily fluid movement into the absorbent 424. The hydrophilic side may also be referred to as a fluid acquisition surface, fluid acquisition side, hydrophilic acquisition surface, or hydrophilic acquisition side. The hydrophilic side may be a fibrous surface and be configured to draw fluid into the upstream layer 426 and the downstream layer 428. In some embodiments, the hydrophilic side of the upstream layer 426 and the downstream layer 428 may be positioned adjacent to the absorbent 424. In other embodiments, the hydrophobic side of the upstream layer 426 and the downstream layer 428 may be positioned adjacent to the absorbent 424. In still other embodiments, the hydrophilic side of one of the upstream layer 426 or the downstream layer 428 may be positioned adjacent to the absorbent 424, and the hydrophobic side of the other of the upstream layer 426 or the downstream layer 428 may be positioned adjacent to the absorbent 424.

In some embodiments, the cover 403 may include or may be a hybrid drape that includes a barrier layer 410, a bonding adhesive layer 412, and a sealing adhesive layer 414. The barrier layer 410 may be formed from a range of medically approved films ranging in thickness from about 15 microns (μm) to about 50 microns (μm). The barrier layer 410 may comprise a suitable material or materials, such as the following: hydrophilic polyurethane (PU), cellulosics, hydrophilic polyamides, polyvinyl alcohol, polyvinyl pyrrolidone, hydrophilic acrylics, hydrophilic silicone elastomers, and copolymers of these. In some embodiments, the barrier layer 410 may be formed from a breathable cast matt polyurethane film sold by Transcontinental Advanced Coatings of Wrexham, United Kingdom, under the name INSPIRE 2301.

The barrier layer 410 may have a high moisture vapor transmission rate (MVTR). The MVTR of the barrier layer 410 allows vapor to egress and inhibits liquids from exiting. In some embodiments, the MVTR of the barrier layer 410 may be greater than or equal to 300 g/m²/24 hours. In other embodiments, the MVTR of the barrier layer 410 may be greater than or equal to 1000 g/m²/24 hours. The illustrative INSPIRE 2301 film may have an MVTR (inverted cup technique) of 14400 g/m²/24 hours and may be approximately 30 microns thick. In other embodiments, a drape having a low MVTR or that allows no vapor transfer might be used. The barrier layer 410 can also function as a barrier to liquids and microorganisms.

In some embodiments, the barrier layer 410 may be adapted to form a bulge on a first side of the barrier layer 410 and a cavity 411 on an opposite side of the barrier layer 410. For example, the barrier layer 410 may be placed on a mold and stretched to plastically deform a portion of the barrier layer 410, forming the cavity 411. A periphery of the barrier layer 410 that is not stretched by the formation of the cavity 411 may form a flange surrounding the cavity 411. In some embodiments, the cavity 411 may be positioned so that a portion of the flange may be larger on a first side of the cavity 411 than on a second side of the cavity 411. The disparity in sizes of the flange may form a foundational flange 430 and a sealing flange 431. In some embodiments, the pouch 405 may be disposed in the cavity 411. The cavity 411 may also be a portion of the barrier layer 410 that is free of the bonding adhesive layer 412. For example, during manufacturing, a portion of the barrier layer 410 may be left without the bonding adhesive layer 412; the area of the barrier layer 410 without the bonding adhesive layer 412 may be equal to a surface area of the pouch 405 to be covered by the barrier layer 410.

The foundational flange 430 may extend away from the cavity 411. In some embodiments, the foundational flange 430 may have a length and a width sufficient to permit other objects to be coupled to the dressing assembly 402. In some embodiments, the foundational flange 430 may support the negative-pressure source 404, as illustrated in FIG. 8.

The bonding adhesive layer 412 may be coupled to the barrier layer 410 on a side of the barrier layer 410 having an opening of the cavity 411. In some embodiments, the bonding adhesive layer 412 may include an aperture 416. The aperture 416 may be coextensive with the opening of the cavity 411. For example, the bonding adhesive layer 412 may cover the barrier layer 410 at the foundational flange 430 and the sealing flange 431, leaving the portion of the barrier layer 410 forming the cavity 411 free of the bonding adhesive layer 412.

The bonding adhesive layer 412 may comprise an acrylic adhesive, rubber adhesive, high-tack silicone adhesive, polyurethane, or other substance. In an illustrative example, the bonding adhesive layer 412 comprises an acrylic adhesive with coating weight of 15 grams/m² (gsm) to 70 grams/m² (gsm). The bonding adhesive layer 412 may be a continuous layer of material or may be a layer with apertures (not shown). The apertures may be formed after application of the bonding adhesive layer 412 or may be formed by coating the bonding adhesive layer 412 in patterns on a carrier layer. In some embodiments, the bond strength of the bonding adhesive may have a peel adhesion or resistance to being peeled from a stainless steel material between about 6N/25 mm to about 40N/25 mm on stainless steel substrate at 23° C. at 50% relative humidity based on the American Society for Testing and Materials (“ASTM”) standard ASTM D3330. The bonding adhesive layer 412 may be about 30 microns to about 60 microns in thickness.

The sealing adhesive layer 414 may be coupled to the bonding adhesive layer 412 and the pouch 405. For example, the sealing adhesive layer 414 may cover the sealing flange 431, the pouch 405, and the foundational flange 430. The sealing adhesive layer 414 may be formed with the plurality of apertures 418. The apertures 418 may be numerous shapes, for example, circles, squares, stars, ovals, polygons, slits, complex curves, rectilinear shapes, triangles, or other shapes. Each aperture 418 of the plurality of apertures 418 may have an effective diameter, which is the diameter of a circular area having the same surface area as the aperture 418. The average effective diameter of each aperture 418 may typically be in the range of about 6 mm to about 50 mm. The plurality of apertures 418 may have a uniform pattern or may be randomly distributed in the sealing adhesive layer 414. Generally, the apertures 418 may be disposed across a length and width of the sealing adhesive layer 414.

The sealing adhesive layer 414 may comprise a silicone gel (or soft silicone), hydrocolloid, hydrogel, polyurethane gel, polyolefin gel, hydrogenated styrenic copolymer gels, or foamed gels with compositions as listed, or soft closed cell foams (polyurethanes, polyolefins) coated with an adhesive (e.g., 40 gsm-70 gsm acrylic), polyurethane, polyolefin, or hydrogenated styrenic copolymers. The sealing adhesive layer 414 may have a thickness in the range of about 100 microns (μm) to about 1000 microns (μm). In some embodiments, the sealing adhesive layer 414 may have stiffness between about 5 Shore 00 and about 80 Shore 00. The sealing adhesive layer 414 may be hydrophobic or hydrophilic. The sealing adhesive of the sealing adhesive layer 414 may be an adhesive having a low to medium tackiness, for example, a silicone polymer, polyurethane, or an additional acrylic adhesive. In some embodiments, the bond strength of the sealing adhesive may have a peel adhesion or resistance to being peeled from a stainless steel material between about 0.5N/25 mm to about 4.5N/25 mm on stainless steel substrate at 23° C. at 50% relative humidity based on ASTM D3330. The sealing adhesive may have a tackiness such that the sealing adhesive may achieve the bond strength above after a contact time of less than 60 seconds. Tackiness may be considered a bond strength of an adhesive after a very low contact time between the adhesive and a substrate. In some embodiments, the sealing adhesive layer 414 may have a tackiness that may be about 40% to about 50% of the tackiness of the bonding adhesive of the bonding adhesive layer 412.

In the assembled state, the bonding adhesive layer 412 may be coupled to the barrier layer 410. The sealing adhesive layer 414 may be coupled to the bonding adhesive layer 412 at the sealing flange 431 and the foundational flange 430 and to the pouch 405 at the cavity 411. In some embodiments, a scrim layer may be disposed in the sealing adhesive layer 414. The scrim layer may provide additional mechanical support for the sealing adhesive layer 414. In some embodiments, the sealing adhesive layer 414 may be treated on a portion and a side of the sealing adhesive layer 414 adjacent to the pouch 405. The treated portion of the sealing adhesive layer 414 may reduce the tackiness of the sealing adhesive layer 414 so that the sealing adhesive layer 414 may not readily adhere to the pouch 405. The initial tackiness of the sealing adhesive layer 414 is preferably sufficient to initially couple the sealing adhesive layer 414 to the epidermis by forming sealing couplings. Once in the desired location, a force can be applied to the barrier layer 410 of the cover 403. For example, the user may rub the foundational flange 430 and the sealing flange 431. This action can cause at least a portion of the bonding adhesive layer 412 to be forced into the plurality of apertures 418 and into contact with the epidermis to form bonding couplings. The bonding couplings provide secure, releasable mechanical fixation to the epidermis.

The average effective diameter of the plurality of apertures 418 for the sealing adhesive layer 414 may be varied as one control of the tackiness or adhesion strength of the cover 403. In this regard, there is interplay between three main variables for each embodiment: the thickness of the sealing adhesive layer 414, the average effective diameter of the plurality of apertures 418, and the tackiness of the bonding adhesive layer 412. The more bonding adhesive of the bonding adhesive layer 412 that extends through the apertures 418, the stronger the bond of the bonding coupling. The thinner the sealing adhesive layer 414, the more bonding adhesive of the bonding adhesive layer 412 generally extends through the apertures 418 and the greater the bond of the bonding coupling. As an example of the interplay, if a very tacky bonding adhesive layer 412 is used and the thickness of the sealing adhesive layer 414 is small, the average effective diameter of the plurality of apertures 418 may be relatively smaller than apertures 418 in a thicker sealing adhesive layer 414 and a less tacky bonding adhesive layer 412. In some embodiments, the thickness of the sealing adhesive layer 414 may be approximately 200 microns, the thickness of the bonding adhesive layer 412 is approximately 30 microns with a tackiness of 2000 g/25 cm wide strip, and the average effective diameter of each aperture 418 is approximately about 6 mm.

As illustrated in FIG. 8, the negative-pressure source 404, which may also be referred to as a blister, may be coupled to the barrier layer 410 of the foundational flange 430. The negative-pressure source 404 may include a barrier layer and a biasing member, for example, a film layer 432, a first foam block 434, a second foam block 435, and a third foam block 437. In some embodiments, the film layer 432 may form a source flange 436 and a source cavity 438. The source cavity 438 may be a portion of the film layer 432 that is plastically deformed, such as by vacuum forming, thermoforming, micro-thermoforming, injection molding, or blow molding, for example. In some embodiments, the source cavity 438 may form walls of the negative-pressure source 404 that may be resilient or flexible. The source flange 436 may be a portion of the film layer 432 adjacent to and surrounding an opening of the source cavity 438. In some embodiments, the first foam block 434, the second foam block 435, and the third foam block 437 may be disposed in the source cavity 438. For example, the first foam block 434, the second foam block 435, and the third foam block 437 may be stacked over one another and positioned within the source cavity 438. The source flange 436 may be coupled to the barrier layer 410 of the foundational flange 430 to seal the first foam block 434, the second foam block 435, and the third foam block 437 in the source cavity 438. In some embodiments, the source flange 436 may be coupled to the barrier layer 410 by high frequency welding, ultrasonic welding, heat welding, or impulse welding, for example. In other exemplary embodiments, the source flange 436 may be coupled to the barrier layer 410 by bonding or folding, for example. In some embodiments, if the source flange 436 is coupled to the barrier layer 410 of the foundational flange 430, the source cavity 438 may be fluidly isolated from the ambient environment and the pouch 405.

The film layer 432 may be constructed from a material that can provide a fluid seal between two components or two environments, such as between the source cavity 438 and a local external environment, while allowing for repeated elastic deformation of the film layer 432. The film layer 432 may be, for example, an elastomeric film or membrane that can provide a seal between the source cavity 438 and the ambient environment. In some example embodiments, the film layer 432 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. In an exemplary embodiment, the film layer 432 may be a polyurethane having a thickness between about 50 microns and about 250 microns and preferably about 100 microns.

The first foam block 434, the second foam block 435, and the third foam block 437 may have similar dimensions. For example, if the first foam block 434, the second foam block 435, and the third foam block 437 are cylindrical, the first foam block 434, the second foam block 435, and the third foam block 437 may have similar diameters. The first foam block 434, the second foam block 435, and the third foam block 437 may be a foam having a plurality of interconnected flow channels. For example, cellular foam, open-cell foam, reticulated foam, porous tissue collections, and other porous material that generally include pores, edges, and/or walls adapted to form interconnected fluid pathways. Liquids, gels, and other foams may also include or be cured to include apertures and flow channels. In some illustrative embodiments, the first foam block 434, the second foam block 435, and the third foam block 437 may be a porous foam material having interconnected cells or pores adapted to uniformly (or quasi-uniformly) distribute fluid throughout the first foam block 434, the second foam block 435, and the third foam block 437. The foam material may be either hydrophobic or hydrophilic. In one non-limiting example, the first foam block 434, the second foam block 435, and the third foam block 437 may be an open-cell, reticulated polyurethane foam such as V.A.C.® GRANUFOAM™ dressing available from Kinetic Concepts, Inc. of San Antonio, Tex. Another exemplary embodiment of the first foam block 434, the second foam block 435, and the third foam block 437 may be Z48AA foam from FXI.

Foam materials may have an elastic modulus, which may also be referred to as a foam modulus. Generally, the elastic modulus of a material may measure the resistance of the material to elastic deformation under a load. The elastic modulus of a material may be defined as the slope of a stress-strain curve in the elastic deformation region of the curve. The elastic deformation region of a stress-strain curve represents that portion of the curve where the deformation of the material due to the applied load is elastic, that is, not permanent. If the load is removed, the material may return to its preloaded state. Stiffer materials may have a higher elastic modulus, and more compliant materials may have a lower elastic modulus. Generally, references to the elastic modulus of a material refers to a material under tension.

For foam materials under compression, the elastic modulus can compared between materials by comparing the compression force deflection (CFD) of the materials. Typically, CFD is determined experimentally by compressing a sample of a material until the sample is reduced to about 25% of its uncompressed size. The load applied to reach the 25% compression of the sample is then divided by the area of the sample over which the load is applied to arrive at the CFD. The CFD can also be measured by compressing a sample of a material to about 50% of the sample's uncompressed size. The CFD of a foam material can be a function of compression level, polymer stiffness, cell structure, foam density, and cell pore size. The first foam block 434, the second foam block 435, and the third foam block 437 may collectively have a CFD of about 4 kPA. The first foam block 434, the second foam block 435, and the third foam block 437 may compress to about 25% of its uncompressed size if a load of about 4 kPa is applied to the first foam block 434, the second foam block 435, and the third foam block 437. Thus, the first foam block 434, the second foam block 435, and the third foam block 437 is more resistant to deformation than the tissue interface 108.

Furthermore, CFD can represent the tendency of a foam to return to its uncompressed state if a load is applied to compress the foam. For example, a foam having a CFD of about 4 kPa may exert about 4 kPa in reaction to 25% compression. The collective CFD of the first foam block 434, the second foam block 435, and the third foam block 437 may represent the ability of the first foam block 434, the second foam block 435, and the third foam block 437 to bias the film layer 432 toward an expanded position. For example, if the first foam block 434, the second foam block 435, and the third foam block 437 is compressed to 25% of its original size, the first foam block 434, the second foam block 435, and the third foam block 437 may collectively exert a spring force that opposes the applied force over the area of the first foam block 434, the second foam block 435, and the third foam block 437 to which the force is applied. The reactive force may be proportional to the amount the first foam block 434, the second foam block 435, and the third foam block 437 are compressed.

In some embodiments, the first foam block 434, the second foam block 435, and the third foam block 437 may have different foam moduli. For example, the first foam block 434 may have a first CFD so that the first foam block 434 may exert a first force when in the compressed state that decreases as the first foam block 434 extends to the uncompressed state. Similarly, the second foam block 435 may have a second CFD so that the second foam block 435 may exert a second force when in the compressed state that decreases as the second foam block 435 extends to the uncompressed state. The third foam block 437 may have a third CFD so that the third foam block 437 may exert a third force when in the compressed state that decreases as the third foam block 437 extends to the uncompressed state. If the first foam block 434, the second foam block 435, and the third foam block 437 are stacked, the first force, the second force, and the third may be combined to reach a total desired spring force. In some embodiments, the CFD of the first foam block 434, the CFD of the second foam block 435, and the CFD of the third foam block 437 may be selected so that the total desired spring force for the combined first foam block 434, the second foam block 435, and the third foam block 437 is generally the same approaching the extended state as in the compressed state. For example, the first foam block 434, the second foam block 435, and the third foam block 437 may be selected so that the collective blocks exert the same upward force over the entire extension of both the first foam block 434, the second foam block 435, and the third foam block 437.

The foam material of the first foam block 434, the second foam block 435, and the third foam block 437 may be selected based on an expected volume of the pouch 405 and the tissue interface 108 (if used). The volume of the pouch 405 may define a volume of fluid to be withdrawn from the pouch 405 to achieve a therapy pressure. For example, if the pouch 405 has a volume of about 50 cubic centimeters, and no tissue interface 108 is used, removing about 10 cubic centimeters of fluid from the pouch 405 may generate a negative pressure of about 125 mm Hg. To generate 125 mm Hg with a single compression of a single foam block having a volume of 10 cm³ the CFD of the single foam block may be around 17 kPa. Similarly, the moduli of the first foam block 434, the second foam block 435, and the third foam block 437 may be selected to have a combined foam modulus of about 17 kPa. Having the first foam block 434, the second foam block 435, and the third foam block 437 may allow for selection of two foams having lower than 17 kPa moduli, which may each be more easily compressed than a single foam having the 17 kPa modulus.

The first foam block 434, the second foam block 435, and the third foam block 437 may have a free volume. The free volume of first foam block 434, the second foam block 435, and the third foam block 437 may be the volume of free space of the first foam block 434, the second foam block 435, and the third foam block 437, for example, the volume of the plurality of channels of the first foam block 434, the second foam block 435, and the third foam block 437. In some embodiments, the free volume of the first foam block 434, the second foam block 435, and the third foam block 437 may be greater than the free volume of the pouch 405. For example, if the free volume of the pouch 405 is 10 cm³, then the free volume of the first foam block 434, the second foam block 435, and the third foam block 437 may be greater than about 20 cm³.

In some embodiments, the negative-pressure source 404 may be fluidly coupled to the cavity 411 through a fluid inlet, such as a tube 440. The tube 440 may be representative of a fluid communication path between the negative-pressure source 404 and the cavity 411. In other embodiments, the tube 440 may be a sealed channel or other fluid pathway. The tube 440 may include a lumen 442 fluidly coupled to the source cavity 438 and the pouch 405. In some embodiments, a valve, such as a check valve 444, may be fluidly coupled to the lumen 442. Exemplary check valves 444 may include ball check valves, diaphragm check valves, swing check valves, stop-check valves, duckbill valves, or pneumatic non-return valves. The check valve 444 may permit fluid communication from the pouch 405 to the source cavity 438 and prevent fluid communication from the source cavity 438 to the pouch 405. For example, if a pressure in the pouch 405 is greater than a pressure in the source cavity 438, the check valve 444 may open, and if the pressure in the source cavity 438 is greater than the pressure in the pouch 405, the check valve 444 may close. In some embodiments, a filter may be disposed on an end of the tube 440. The filter may be a hydrophobic porous polymer filter having gel blocking properties.

The source cavity 438 may also be fluidly coupled to the ambient environment through a fluid outlet, such as a tube 446. For example, the tube 446 having a lumen 448 may fluidly couple the source cavity 438 to the ambient environment. The tube 446 may be representative of a fluid communication path between the ambient environment and the source cavity 438. A valve, such as a check valve 450, may be fluidly coupled to the lumen 448 to control fluid communication through the lumen 448. Exemplary check valves 450 may include ball check valves, diaphragm check valves, swing check valves, stop-check valves, duckbill valves, or pneumatic non-return valves. In some embodiments, the check valve 450 may permit fluid communication from the source cavity 438 to the ambient environment and prevent fluid communication from the ambient environment to the source cavity 438. For example, if a pressure in the source cavity 438 is greater than a pressure in the ambient environment, the check valve 450 may open, and if the pressure in the ambient environment is greater than the pressure in the source cavity 438, the check valve 450 may close. In some embodiments, a filter may be disposed on an end of the tube 446. The filter may be a hydrophobic porous polymer filter having gel blocking properties.

The dressing assembly 402 may be disposed over the tissue site to form the sealed therapeutic environment. In some embodiments, the pouch 405 of the dressing assembly 402 may be positioned over the tissue site and the negative-pressure source 404 may be positioned over undamaged tissue proximate the tissue site. A force, such as hand pressure, may be applied to the sealing flange 431 and the foundational flange 430, urging the bonding adhesive of the bonding adhesive layer 412 through the apertures 418 of the sealing adhesive layer 414 to form bonding couplings and securing the dressing assembly 402 to the tissue site.

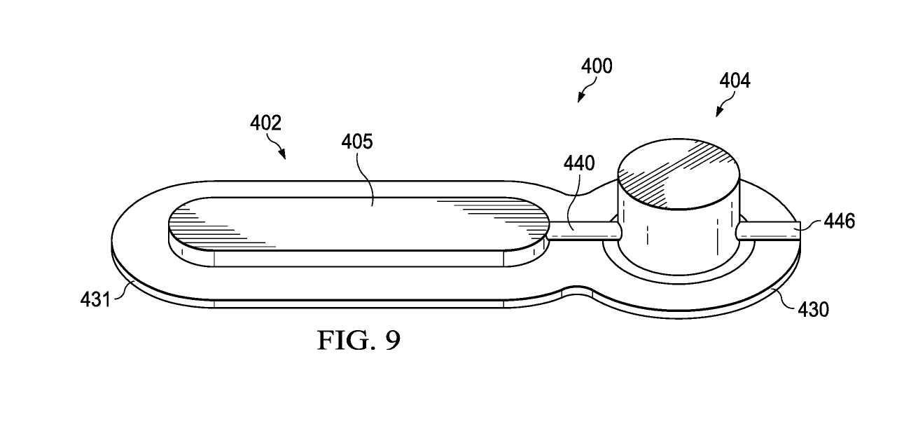

FIG. 9 is a perspective view illustrating additional details of the negative-pressure source 404. Once positioned, the negative-pressure source 404 may be operated to generate a negative pressure in the pouch 405. A force, such as hand pressure, may be applied to the film layer 432 over the first foam block 434, the second foam block 435, and the third foam block 437 to compress the first foam block 434, the second foam block 435, and the third foam block 437 to decrease the volume of the source cavity 438. If the first foam block 434, the second foam block 435, and the third foam block 437 and the source cavity 438 are fluidly isolated from the ambient environment, compression of the first foam block 434, the second foam block 435, and the third foam block 437 may increase the pressure in the source cavity 438. An increase of pressure in the source cavity 438 may create a pressure differential across the check valve 444 that urges the check valve 444 to close. Similarly, an increase of pressure in the source cavity 438 may create a pressure differential across the check valve 450 that urges the check valve 450 to open, allowing fluid from the source cavity 438 to flow through the tube 446 to the ambient environment. If the force is removed, the first foam block 434, the second foam block 435, and the third foam block 437 may expand, increasing the volume of the source cavity 438 and decreasing the pressure in the source cavity 438. In response, the decrease in pressure in the source cavity 438 may create a pressure differential across the check valve 450 that urges the check valve 450 to close, preventing fluid from flowing from the ambient environment to the source cavity 438. The decrease in pressure in the source cavity 438 may also create a pressure differential across the check valve 444 that urges the check valve 444 to open, permitting fluid flow from the pouch 405 to the source cavity 438. Fluid may flow from the pouch 405 to the source cavity 438 until the source cavity 438 and the first foam block 434, the second foam block 435, and the third foam block 437 reach their respective uncompressed positions. In this manner, a portion of the total volume of fluid in the sealed therapeutic environment may be removed. In response to the removal of a portion of the fluid, a smaller volume of fluid occupies the sealed therapeutic environment, decreasing the pressure. Each time the first foam block 434, the second foam block 435, and the third foam block 437 are compressed and allowed to rebound, additional fluid may be removed from the sealed therapeutic environment, further decreasing the pressure.

Decreasing the pressure in the sealed therapeutic environment may create a pressure differential across the dressing assembly 402. If the pressure in the sealed therapeutic environment reaches the therapy pressure for negative-pressure therapy, the CFD of the first foam block 434, the second foam block 435, and the third foam block 437 may be insufficient to cause the first foam block 434, the second foam block 435, and the third foam block 437 to expand following compression of the first foam block 434, the second foam block 435, and the third foam block 437. The therapy pressure may be the pressure at which negative-pressure therapy may be performed. In some embodiments, the therapy pressure provided by the first foam block 434, the second foam block 435, and the third foam block 437 may be about 70 mm Hg of negative pressure. In other embodiments, the therapy pressure provided by the first foam block 434, the second foam block 435, and the third foam block 437 may be between about 50 mm Hg and 150 mm Hg of negative pressure. If the first foam block 434, the second foam block 435, and the third foam block 437 remains compressed, a patient or clinician may have an indication that the therapy pressure has been reached. The compressed first foam block 434, the second foam block 435, and the third foam block 437 may also act as a pressure reservoir. As negative-pressure therapy is provided, there may be a natural leakage or decline of negative pressure at the tissue site. As the negative pressure decreases in the sealed therapeutic environment, the pressure differential across the dressing assembly 402 may decrease and the first foam block 434, the second foam block 435, and the third foam block 437 may gradually expand, reapplying negative pressure at the tissue site. In some embodiments, the negative-pressure source 404 having the first foam block 434, the second foam block 435, and the third foam block 437 may maintain a therapeutic negative pressure for about 8 hours or more.

In some embodiments, the fluid container and dressing assembly may be shaped to accommodate differently shaped tissue sites. For example, the pouch 105 and the dressing assembly 102 of FIG. 1-3 and the pouch 205 and the dressing assembly 202 of FIG. 4 may have a square shape and a large area to accommodate a tissue site having a large area. The pouch 305 and the dressing assembly 302 of FIG. 5, FIG. 6, and FIG. 7 may have a curved shape to accommodate wounds having a significant curvature or that may be located on or near an articulating joint. The pouch 405 and the dressing assembly 402 of FIG. 8 and FIG. 9 may have a rectangular shape to accommodate a tissue site, such as a linear wound, that has a high length to width ratio.

In some embodiments, the foam block 134, 234, 334, 335, 434, 435, 437 may be replaced with other types of elastic elements, such as a polymer coil spring formed of polyurethane or acrylonitrile butadiene styrene (ABS). In some embodiments, the negative-pressure source 104, 204, 304, and 404 may comprise or may be a blow-molded bellows that is coupled to the foundational flange 130, 230, 330, or 430.

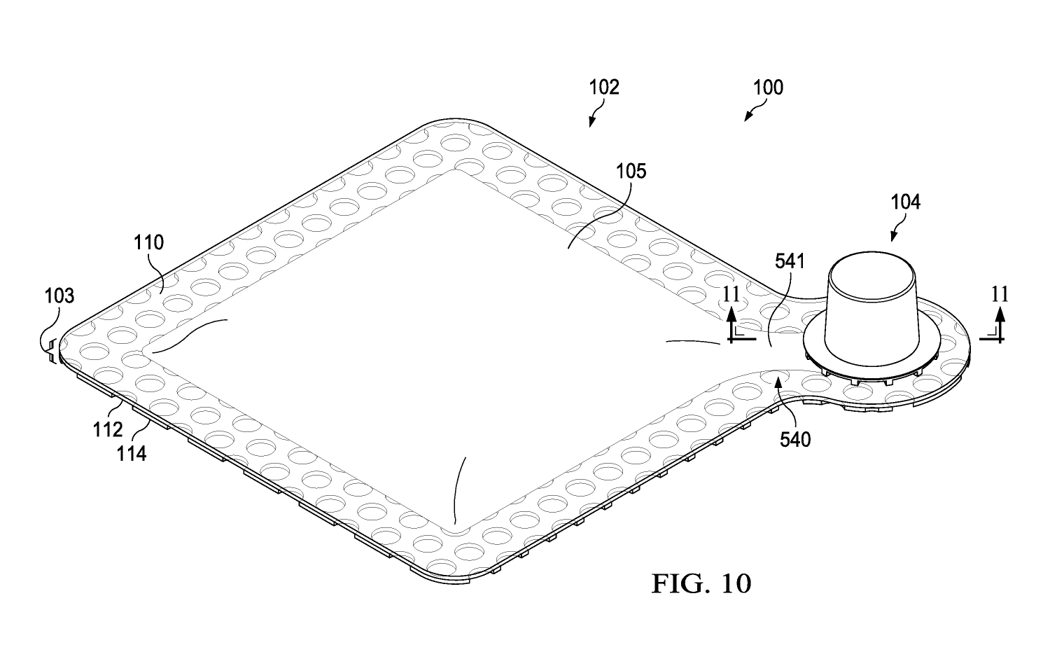

FIG. 10 is a top perspective view illustrating additional details of an alternative embodiment of the negative-pressure therapy system 100. The negative-pressure therapy system 100 can include the dressing assembly 102, the cover 103, the negative-pressure source 104, the pouch 105, and a conduit 540. The conduit 540 may be similar to and operate as described above with respect to the tube 140 of FIG. 1. In some embodiments, the conduit 540 may be a portion of the cover 103. For example, the cover 103 may have a square shape having four corners. The cover 103 can include a projection 541. The projection 541 may extend from a corner of the cover 103. In some embodiments, the projection 541 can extend from the pouch 105 to the negative-pressure source 104. The projection 541 may be an integral component of the cover 103 having a similar thickness and being formed from a similar material. The conduit 540 can have an open cross-sectional area through which fluid can flow between about 20 mm² and about 22 mm². In some embodiments, the cross-sectional area of the conduit 540 can be about 21.87 mm².

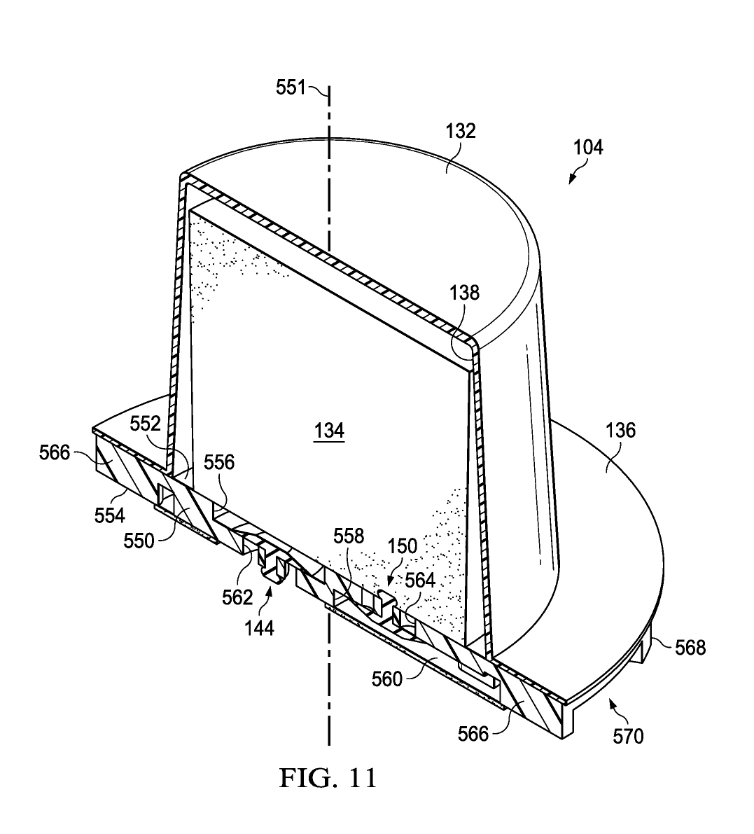

FIG. 11 is a sectional view taken along line 11-11 of FIG. 10 illustrating an alternative negative-pressure source 104 that can be used with the negative-pressure therapy system 100. The negative-pressure source 104 can include the film layer 132, the foam block 134, the source flange 136, and the source cavity 138. In some embodiments, the foam block 134 can be a cylinder formed from a reticulated polyurethane foam having approximately 45 pores per inch (“ppi”). In other embodiments, the foam block 134 can be formed from a reticulated polyurethane foam having approximately 80 ppi, a felted foam having a firmness factor of 5, a modified felted foam, a modified closed cell foam, or a thermoplastic honeycomb cellular matrix. The foam block 134 can be disposed in the source cavity 138.

A felted foam is a foam that undergoes a thermoforming process to permanently compress the foam to increase the density of the foam. A felted foam may also be compared to other felted foams or compressed foams by comparing a firmness factor of the felted foam to the firmness factor of other compressed or uncompressed foams. Generally a compressed or felted foam may have a firmness factor greater than 1. A firmness factor (FF) is defined as a ratio of the density of a foam in a compressed state to the density of the same foam in an uncompressed state. For example, a firmness factor (FF) of 5 may refer to a compressed foam having a density that is five times greater than a density of the same foam in an uncompressed state. Mechanically or chemically compressing a foam may reduce a thickness of the foam at ambient pressure when compared to the same foam that has not been compressed. Reducing a thickness of a foam by mechanical or chemical compression may increase a density of the foam, which may increase the firmness factor (FF) of the foam. Increasing the firmness factor (FF) of a foam may increase a stiffness of the foam in a direction that is parallel to a thickness of the foam. A thermoplastic honeycomb cellular matrix may have an open area or void space percentage of 90%. In some embodiments, the thermoplastic honeycomb cellular matrix can be a fusion bonded matrix produced by Supracor Inc. of San Jose, Calif.

The negative-pressure source 104 can include a base 550. The base 550 may be a disc-shaped body having an axis 551, an upper surface or first surface 552, and a lower surface or second surface 554. In some embodiments, the base 550 can have a peripheral ring 566. The first surface 552 can be flush across the peripheral ring 566 and the base 550. In some embodiments, the peripheral ring 566 can be thicker than the base 550 so that a second surface of the peripheral ring 566 that is proximate to the second surface 554 may have a different elevation than the second surface 554 of the base 550. In some embodiments, the peripheral ring 566 may be integral to the base 550. In other embodiments, the peripheral ring 566 may be a separate component coupled to the base 550 by, for example, welding, adhering, fusing, or otherwise securing the peripheral ring 566 to the base 550. The base 550 can be formed from an elastomeric material, skinned foam, or closed cell foam.

The base 550 can have an inlet channel or a first cavity 556 and an exhaust channel or a second cavity 558. The first cavity 556 can be disposed in the first surface 552, and the second cavity 558 can be disposed in the second surface 554. In some embodiments, the first cavity 556 and the second cavity 558 can be circumferentially disposed about the axis 551. The first cavity 556 can include an inlet recess such as a first bore 562, and the second cavity 558 can include an exhaust recess such as a second bore 564. The first bore 562 can be disposed proximate a center of the first cavity 556 and extend from the first cavity 556 through the second surface 554. The first bore 562 can permit fluid communication across the base 550. Similarly, the second bore 564 can be disposed proximate a center of the second cavity 558 and extend from the second cavity 558 through the first surface 552. The second bore 564 can permit fluid communication across the base 550. The second cavity 558 may include at least one channel 560 extending from the second cavity 558 to the periphery of the base 550. In some embodiments, the channel 560 can permit fluid communication between the second cavity 558 and the ambient environment. In some embodiments, the first cavity 556 and the second cavity 558 can have a cross sectional area between about 20 mm² and about 22 mm². For example, the first cavity 556 and the second cavity 558 can have a cross sectional area of about 21.87 mm².

The source flange 136 can be coupled to the base 550 to enclose the source cavity 138 between the film layer 132 and the base 550. In some embodiments, the source flange 136 can be coupled to the first surface 552 over the peripheral ring 566 to enclose the source cavity 138 between the base 550 and the film layer 132. In some embodiments, the film layer 132 can form a flexible side wall of the negative-pressure source 104. A periphery of the source flange 136 can be coincident with a periphery of the peripheral ring 566 so that adjacent edges of the source flange 136 and the first surface 552 are flush.

The check valve 144 can be disposed in the first cavity 556 and be oriented to permit fluid communication from the second surface 554 of the base 550 into the source cavity 138. The check valve 150 can be disposed in the second cavity 558 and be oriented to permit fluid communication from the source cavity 138 to the second surface 554 of the base 550. In some embodiments, the check valve 144 and the check valve 150 can be umbrella valves, flap valves, duckbill valves, diaphragm valves, or sprung loaded ball valves. The base 550 can provide shielding for the check valve 144 and the check valve 150 to prevent the foam block 134 from interfering with the check valve 144 and the check valve 150. In some embodiments, the check valve 144 and the check valve 150 can be umbrella valves, such as a VL2501-102 formed from silicone having a mean cracking pressure of 9.1 mbar produced by Vernay Flow Control Solutions.

Shielding can refer to the protection of the operation of the check valve 144 and the check valve 150 during compression of the foam block 134. For example, the base 550 can have a wall or lip around the check valve 144 and the check valve 150 to stop the foam from interfering with the valve operation. For example, by forming the first cavity 556 and the second cavity 558 so that the check valve 144 and the check valve 150 are recessed from the first surface 552 and the second surface 554. In other embodiments, the check valve 144 and the check valve 150 can be shielded by positioning a polyester material over the check valve 144 and the check valve 150. In some embodiments, the polyester material can be clear and have a thickness of about 0.05 mm.

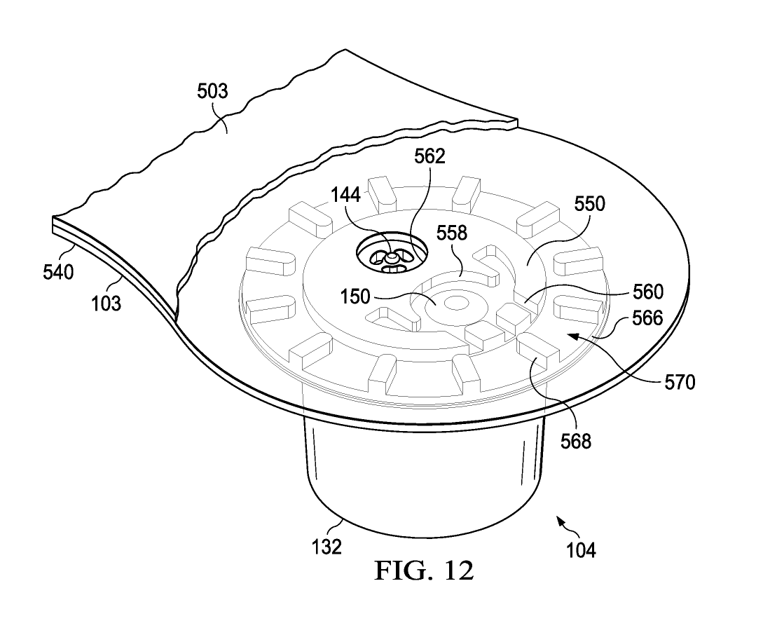

FIG. 12 is a bottom perspective view illustrating additional details that may be associated with the negative-pressure source 104 of FIG. 10. In some embodiments, the peripheral ring 566 can include a plurality of channels 570 separated by a plurality of standoffs 568. The plurality of channels 570 and the plurality of standoffs 568 can be circumferentially disposed around the peripheral ring 566. In some embodiments, the conduit 540 may further comprise an enclosing layer 503. The enclosing layer 503 can be coupled at its periphery to the projection 541 of the cover 103 to enclose a free volume. The free volume may be fluidly coupled to the pouch 105 and the negative-pressure source 104. In some embodiments, the enclosing layer 503 can be coupled to the base 550 so that the first cavity 556 is fluidly coupled to the free volume of the conduit 540. Preferably, the enclosing layer 503 can seal the conduit 540 from the ambient environment, permitting a pressure other than ambient pressure to be maintained in the conduit 540 and communicated between the pouch 105 and the negative-pressure source 104 through the first bore 562.

In operation, the foam block 134 can be compressed. In response, the source cavity 138 is decreased in volume and fluid within the source cavity 138 can be exhausted to the ambient environment through the check valve 150 disposed in the second cavity 558, through the channel 560 and the plurality of channels 570. If the compressive force is removed, the foam block 134 can expand, increasing the volume of the source cavity 138. In response, the check valve 144 may be opened in response to the differential pressure between the source cavity 138 and the pouch 105. Fluid can flow from the pouch 105 through the conduit 540, the check valve 144, the first cavity 556, and into the source cavity 138, generating a negative pressure in the pouch 105. Subsequent compression of the foam block 134 can draw additional fluid from the pouch 105, increasing the negative pressure within the pouch 105 until the reactive force of the foam block 134 acting to inflate the source cavity 138 is less than the negative pressure within the pouch 105. In some embodiments, the negative-pressure source 104 can generate around 100 mm Hg of negative pressure within the pouch 105.

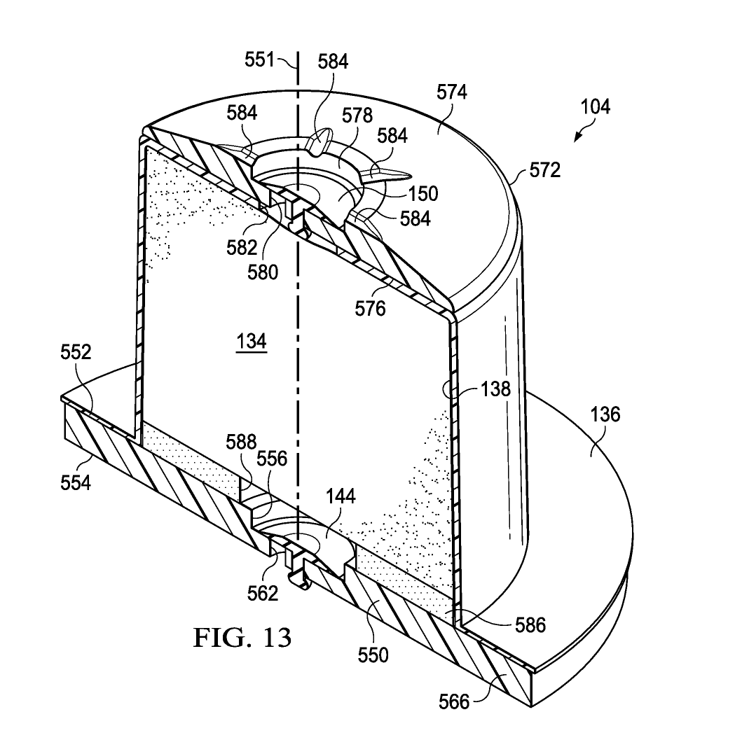

FIG. 13 is a sectional view illustrating additional details of another embodiment of the negative-pressure source 104. The negative-pressure source 104 of FIG. 13 may be similar to the negative-pressure source 104 of FIG. 11 and FIG. 12. In alternative embodiments, the base 550 can have the first cavity 556 without the second cavity 558. The first cavity 556 can be disposed in the first surface 552. The first cavity 556 can be concentric with the axis 551. The first cavity 556 can include the first bore 562. The first bore 562 can be disposed proximate a center of the first cavity 556 and extend from the first cavity 556 through the second surface 554. The first bore 562 can permit fluid communication across the base 550. The check valve 144 can be disposed in the first cavity 556 and be oriented to permit fluid communication from the second surface 554 of the base 550 into the source cavity 138.

As shown in FIG. 13, the negative-pressure source 104 can include a cap 572. The cap 572 can be positioned opposite the base 550 and disposed over the foam block 134. The cap 572 can have a first surface 574 and a second surface 576 that is opposite the first surface 574. The second surface 576 can be adjacent to the film layer 132. In other embodiments, the second surface 576 can be adjacent to the foam block 134 and the first surface 574 can be adjacent to the film layer 132. In some embodiments, the cap 572 can have a cavity 578 depending into the cap 572 from the first surface 574 toward the second surface 576. The cavity 578 can depend into the cap 572 about half the thickness of the cap 572. In some embodiments, the cavity 578 can be disposed proximate to a center of the cap 572. The cap 572 can have a first thickness adjacent to the cavity 578 and a second thickness at a periphery of the cap 572. In some embodiments, the first surface 574 may taper from the first thickness to the second thickness. For example, the first thickness may be greater than the second thickness and the first surface 574 may taper from the first thickness to the second thickness. In some embodiments, the cap 572 can have a plurality of notches 584. The plurality of notches 584 can be circumferentially disposed around the cavity 578. In some embodiments, each notch 584 can have a primary dimension orienting the notch 584 from the cavity 578 toward the periphery of the cap 572. In some embodiments, the notches 584 can provide uninterrupted or relatively uninterrupted air flow if the cap 572 is covered, for example, by a hand. In some embodiment, the collective free cross sectional area formed by the notches 584 can be between about 20 mm² and about 22 mm². For example, the notches can form a free a cross sectional area of about 21.87 mm².

A bore 580 can be disposed in the cap 572. In some embodiments, the bore 580 can be positioned in the cavity 578 and extend from the first surface 574 to the second surface 576, permitting fluid communication across the cap 572 through the bore 580 and the cavity 578. In some embodiments, the film layer 132 can have an aperture 582. The aperture 582 may have an average effective diameter that is less than an average effective diameter of the cap 572. The aperture 582 can permit fluid communication with the source cavity 138 across the film layer 132. The cap 572 can be coupled to the film layer 132 adjacent to the aperture 582, for example, by welding, adhering, bonding, or otherwise securing the cap 572 to the film layer 132. In some embodiments, the bore 580 can be disposed over and in fluid communication with the aperture 582.

The check valve 150 can be disposed in the bore 580 of the cavity 578. In some embodiments, the check valve 150 can be an umbrella valve, a flap valve, a duckbill valve, a diaphragm valve, or a sprung loaded ball valve. In some embodiments, the check valve 150 permits fluid communication from the source cavity 138 to the ambient environment and prevents fluid communication from the ambient environment into the source cavity 138. In some embodiments, the cavity 578 can shield the check valve 150 by separating the check valve 150 from an exterior surface of the cap 572.

In some embodiments, a spacer 586 can be disposed in the source cavity 138. The spacer 586 may be positioned between the foam block 134 and the base 550. The spacer 586 can be formed from an elastomeric or other similar material. In some embodiments, the spacer 586 can include an opening 588. The opening 588 can have an average effective diameter greater than the average effective diameter of the first cavity 556. In other embodiments, the base 550 can have an increased thickness to form a boss substantially filling a lower portion of the source cavity 138.

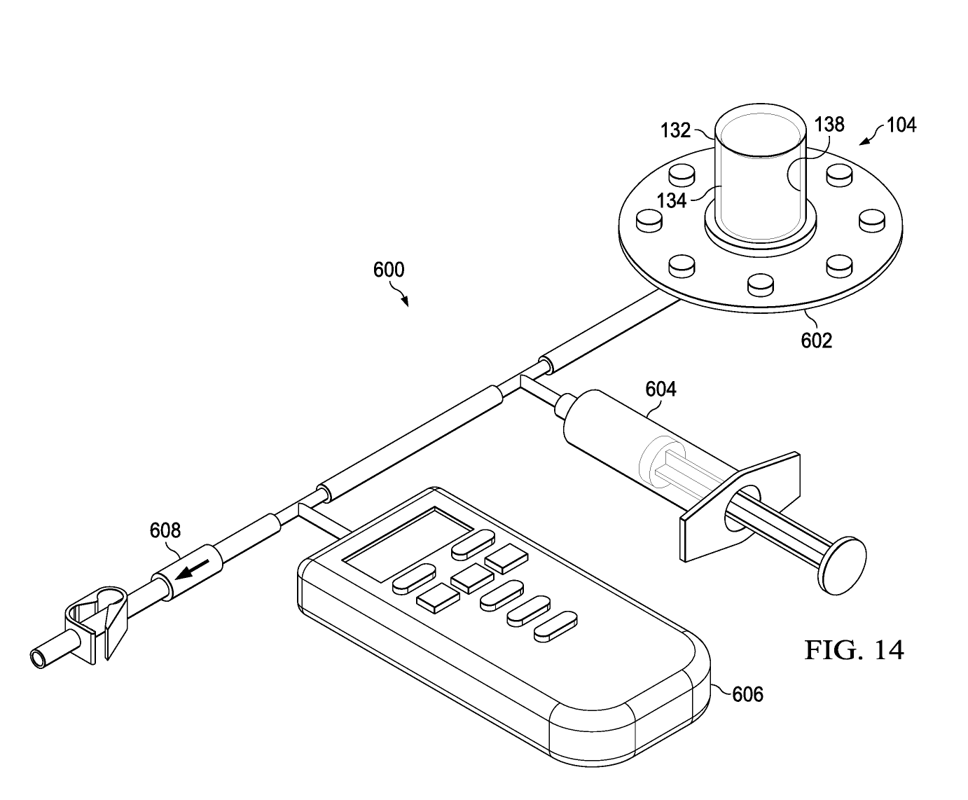

FIG. 14 is a perspective view illustrating additional details of a testing apparatus 600 that may be associated with some embodiments. The testing apparatus 600 can include a receiver 602, a plenum 604, a pressure tester 606, and a valve 608. Each of the components can be fluidly coupled by one or more conduits or other fluid connectors. The receiver 602 can be a device configured to provide a re-sealable source chamber for a biasing member to be tested. For example, the receiver 602 can be a pair of separable plates configured to receive the film layer 132 and the foam block 134 and seal around the source flange 136 to form the source cavity 138. The receiver 602 can position the foam block 134 and the film layer 132 so that the foam block 134 and the film layer 132 can be compressed by hand. The receiver 602 provides a mechanism to fluidly couple the source cavity 138 to other components. The plenum 604 may be a reservoir of fluid, for example, about 40 milliliters (mL). In some embodiments, the plenum 604 may be a syringe. In alternative embodiments, the plenum 604 may be omitted. The pressure tester 606 can be a 2022 Pressure Tester rated for 0 to 1500 mm Hg produced by Sifam Instruments capable of measuring pressure in a system.

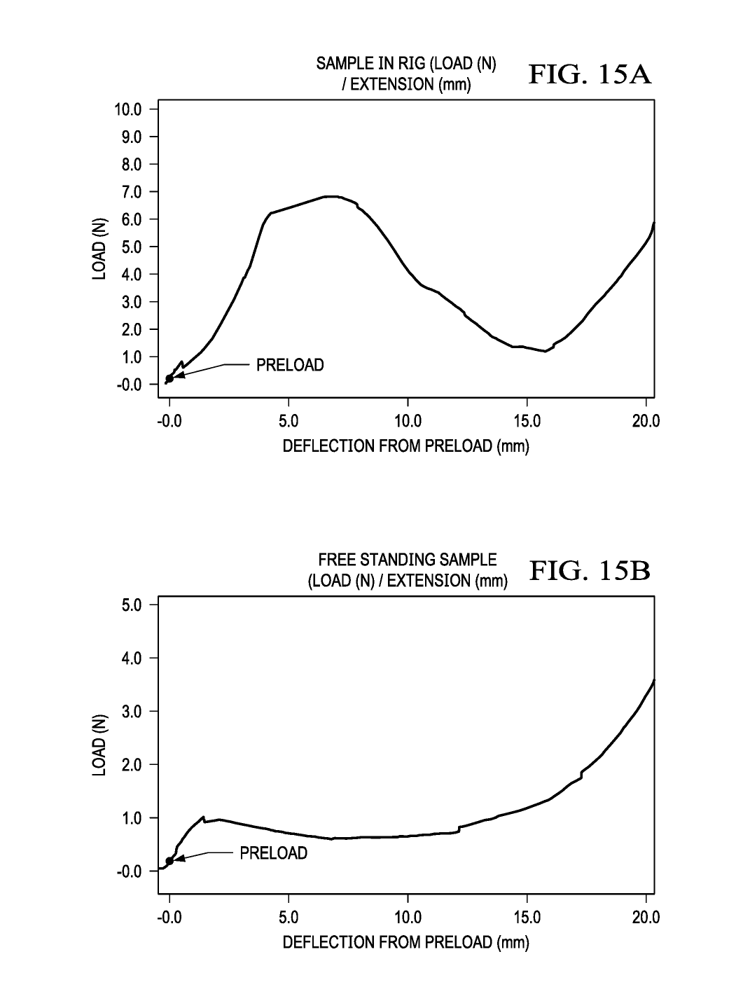

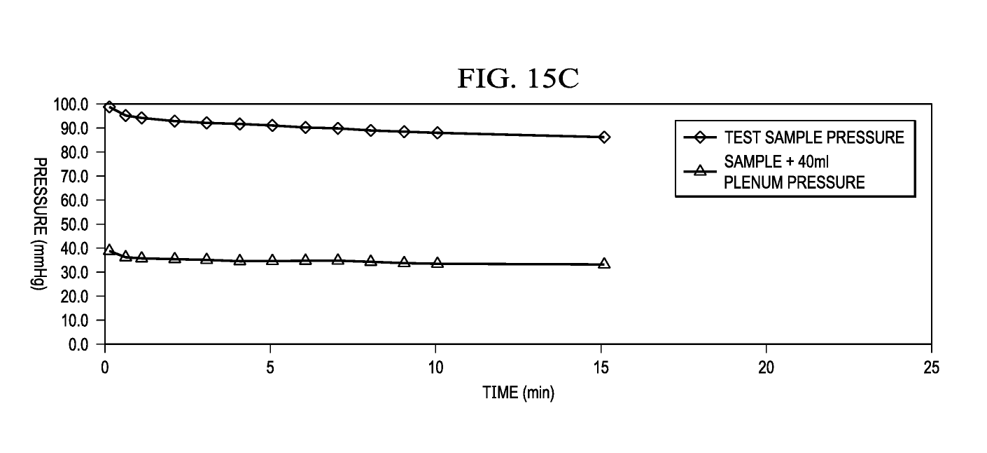

FIG. 15A is a line graph illustrating a load in Newtons (N) versus a deflection from preload in millimeters (mm) for a biasing member of the negative-pressure therapy system of FIG. 10. In particular, FIG. 15A illustrates a load in Newtons applied to the foam block 134 having a 0.3N preload to produce the corresponding deflection of the foam block 134 from its preloaded position where the testing is conducted in the testing apparatus 600 of FIG. 14. FIG. 15B is a line graph illustrating a load in Newtons (N) versus a deflection from preload in millimeters (mm) for a free standing embodiment of the biasing member of FIG. 15A. In FIG. 15A and FIG. 15B the foam block 134 was formed from a V.A.C.® GRANUFOAM™ dressing. The foam block 134 was cylindrical in shape having a diameter of about 40 mm, a height of about 30 mm, and a volume of about 37.7 cubic centimeters (“cc”). The foam block 134 was formed from a single layer. Table 01 illustrates the applied force to achieve the distance deflection.

TABLE 01

| Distance (mm) | Test Rig | Free Standing |
|---|---|---|
| 5 | 6.40 | 0.72 |
| 10 | 3.99 | 0.67 |
| 15 | 1.29 | 1.20 |
| 20 | 5.01 | 3.37 |
| 25 | — | 19.85 |
| 30 | — | — |
| Distance @ 20N: | 24.50 | 25.20 |
| Average (N): | 4.17 | 5.16 |

As illustrated in Table 01, less load was used to compress the free standing sample to the same deflection as the sample in the rig. The free standing sample can provide a baseline for the material average force of the sample.

FIG. 15C is a line graph illustrating a pressure in millimeters mercury (mm Hg) versus time in minutes for the biasing members of FIG. 15A and FIG. 15B. The biasing member of FIG. 15A and FIG. 15B was tested in a first scenario without the plenum 604 and in a second scenario with the plenum 604. Table 02 illustrates the change in pressure over time.

TABLE 02
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 98.6 | | 38.6 | |
| 0.5 | 95.0 | 3.6 | 36.0 | 2.6 |
| 1 | 93.9 | 1.1 | 35.7 | 0.3 |
| 2 | 92.8 | 1.1 | 35.2 | 0.5 |
| 3 | 92.0 | 0.8 | 35.1 | 0.1 |
| 4 | 91.7 | 0.3 | 34.5 | 0.6 |
| 5 | 91.1 | 0.6 | 34.4 | 0.1 |
| 6 | 90.3 | 0.8 | 34.7 | −0.3 |
| 7 | 90.0 | 0.3 | 34.7 | 0 |
| 8 | 89.4 | 0.6 | 34.5 | 0.2 |
| 9 | 88.9 | 0.5 | 34.2 | 0.3 |
| 10 | 88.5 | 0.4 | 33.9 | 0.3 |
| 15 | 87.4 | 1.1 | 33.9 | 0 |
| 20 | | N/A | | N/A |
| 25 | | N/A | | N/A |

| | | |
|---|---|---|
| Avg. change | 0.93 | 0.39 |
| (mmHg) | | |

For each biasing member, the starting pressure is 0 mm Hg. As illustrated in Table 02, the biasing member was able to generate a higher negative pressure without the 40 mL plenum. The biasing member tested with the plenum and the biasing member testing without the plenum were able to maintain the negative pressure within about 10% of the initially developed pressure.

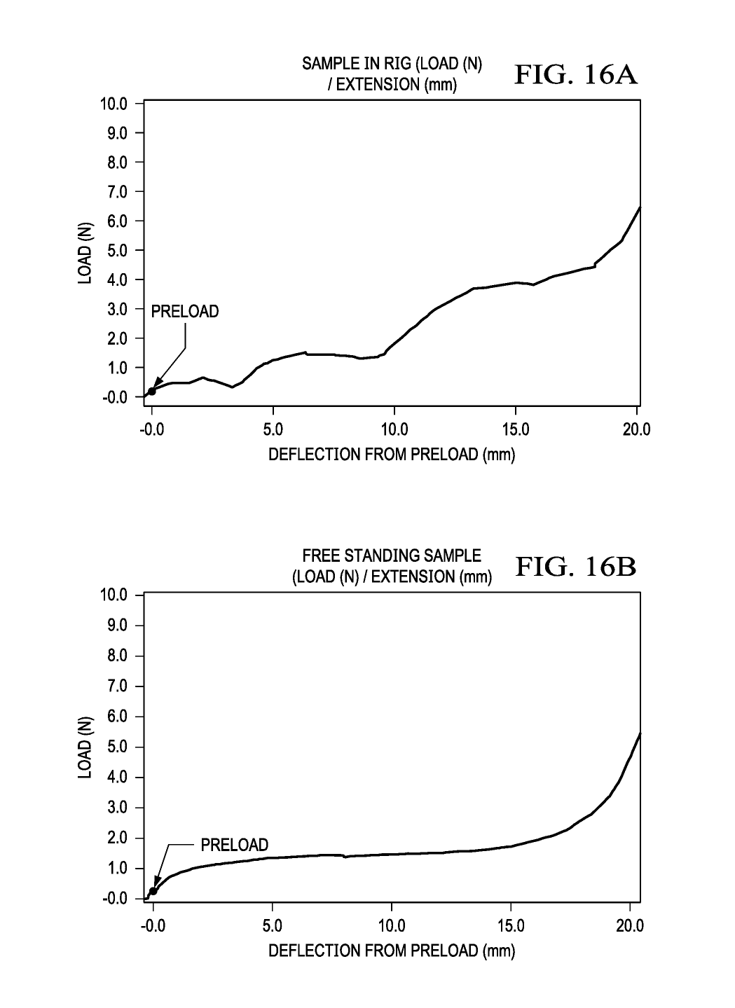

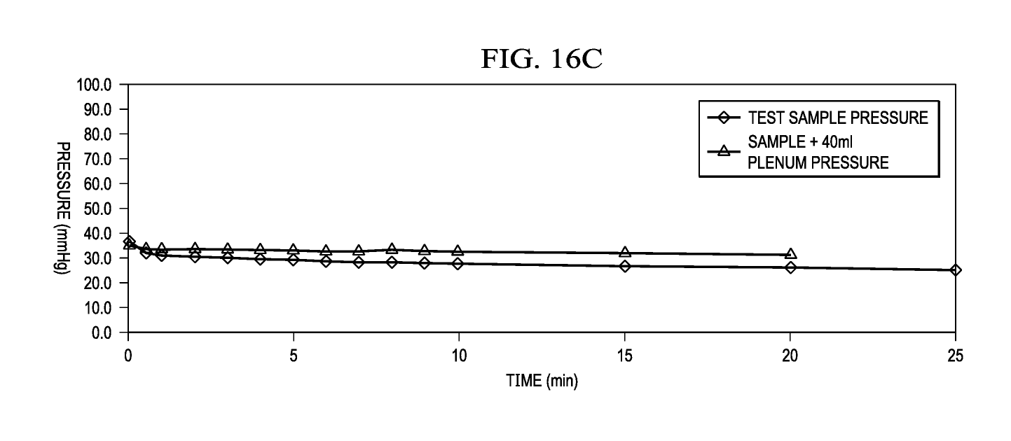

FIG. 16A is a line graph illustrating a load in Newtons (N) versus a deflection from preload in millimeters (mm) for a biasing member of the negative-pressure therapy system of FIG. 10. In particular, FIG. 16A illustrates a load in Newtons applied to the foam block 134 to produce the corresponding deflection of the foam block 134 from its preloaded position where the testing is conducted in the testing apparatus 600 of FIG. 14. FIG. 16B is a line graph illustrating a load in Newtons (N) versus a deflection from preload in millimeters (mm) for a free standing embodiment of the biasing member of FIG. 16A. In FIG. 16A and FIG. 16B, the foam block 134 was formed from a reticulated foam having 80 pores per inch (“ppi”). The foam block 134 was cylindrical in shape having a diameter of about 40 mm, a height of about 26 mm, and a volume of about 32.7 cubic centimeters (“cc”). The foam block 134 was formed from two layers each having a height of about 13 mm. The preload was about 0.3 N. Table 03 illustrates the applied force to achieve the distance deflection.

TABLE 03

| Distance (mm) | Test Rig | Free Standing |
|---|---|---|
| 5 | 1.28 | 1.34 |
| 10 | 1.88 | 1.49 |
| 15 | 3.95 | 1.82 |
| 20 | 6.27 | 4.96 |
| 25 | — | — |
| 30 | — | — |
| Distance @ 20N: | 22.60 | 23.20 |
| Average (N): | 3.35 | 2.40 |

As illustrated in Table 03, less load was used to compress the free standing sample to the same deflection as the sample in the rig.

FIG. 16C is a line graph illustrating a pressure in millimeters mercury (mm Hg) versus time in minutes for the biasing members of FIG. 16A and FIG. 16B. The biasing member of FIG. 16A and FIG. 16B was tested in a first scenario without the plenum 604 and in a second scenario with the plenum 604. Table 04 illustrates the change in pressure over time.

TABLE 04
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 36.3 | | 35.9 | |
| 0.5 | 31.8 | 4.5 | 34.2 | 1.7 |
| 1 | 31.0 | 0.8 | 34.2 | 0 |
| 2 | 30.3 | 0.7 | 34.0 | 0.2 |
| 3 | 30.0 | 0.3 | 34.1 | −0.1 |
| 4 | 29.7 | 0.3 | 34.0 | 0.1 |
| 5 | 29.6 | 0.1 | 34.2 | −0.2 |
| 6 | 29.1 | 0.5 | 33.9 | 0.3 |
| 7 | 28.8 | 0.3 | 34.0 | −0.1 |
| 8 | 28.8 | 0 | 34.2 | −0.2 |
| 9 | 28.6 | 0.2 | 34.2 | 0 |
| 10 | 28.4 | 0.2 | 33.9 | 0.3 |
| 15 | 28.1 | 0.3 | 33.8 | 0.1 |
| 20 | 28.0 | 0.1 | 33.8 | 0 |
| 25 | 27.4 | 0.6 | | |

| | | |
|---|---|---|
| Avg. change | 0.64 | 0.16 |
| (mmHg) | | |

As illustrated in Table 04, the biasing member was able to generate a higher negative pressure without the 40 mL plenum. The biasing member tested without the plenum maintained the negative pressure within about 24% of the initially developed pressure. The biasing member testing with the plenum were able to maintain the negative pressure within about 6% of the initially developed pressure.

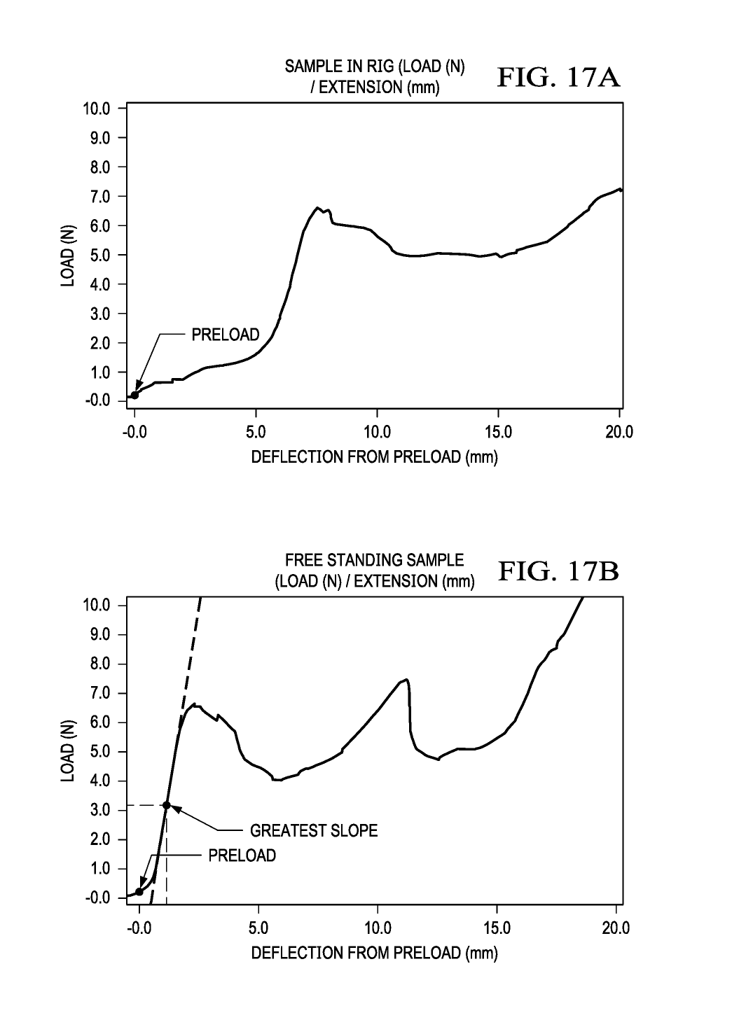

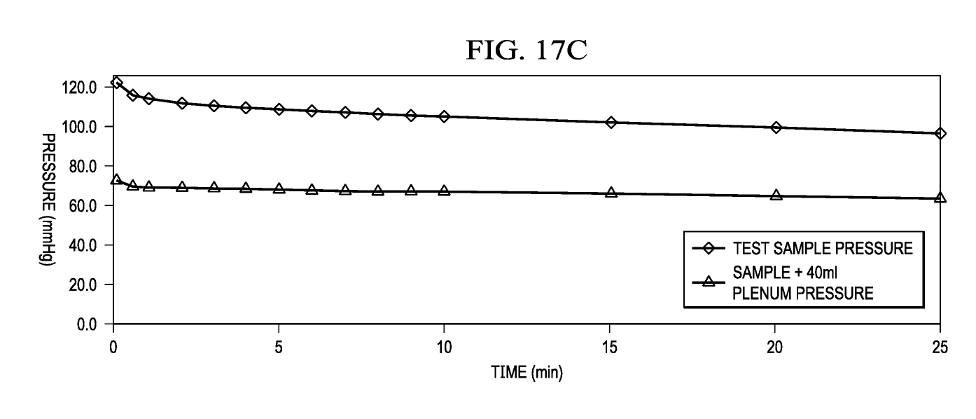

FIG. 17A is a line graph illustrating a load in Newtons (N) versus a deflection from preload in millimeters (mm) for a biasing member of the negative-pressure therapy system of FIG. 10. In particular, FIG. 17A illustrates a load in Newtons applied to the foam block 134 to produce the corresponding deflection of the foam block 134 from its preloaded position where the testing is conducted in the testing apparatus 600 of FIG. 14. FIG. 17B is a line graph illustrating a load in Newtons (N) versus a deflection from preload in millimeters (mm) for a free standing embodiment of the biasing member of FIG. 17A. In FIG. 17A and FIG. 17B, the foam block 134 was formed from a blue honeycomb, for example, a thermoplastic (TPE) fusion bonded honeycomb cellular matrix having 90% open or void space produced by Supracor, Inc. of San Jose, Calif. The foam block 134 was cylindrical in shape having a diameter of about 40 mm, a height of about 28 mm, and a volume of about 30.1 cubic centimeters (“cc”). The foam block 134 was formed from two layers each having a height of about 14 mm. The preload was about 0.3 N. Table 05 illustrates the applied force to achieve the distance deflection.

TABLE 05

| Distance (mm) | Test Rig | Free Standing |
|---|---|---|
| 5 | 1.56 | 4.52 |
| 10 | 5.67 | 6.45 |
| 15 | 4.98 | 5.55 |
| 20 | 7.34 | 13.09 |
| 25 | 13.34 | — |
| 30 | — | — |
| Distance @ 20 N: | 27.10 | 22.10 |
| Average (N): | 6.58 | 7.40 |

As illustrated in Table 05, less load was used to compress the free standing sample to the same deflection as the sample in the rig.

FIG. 17C is a line graph illustrating a pressure in millimeters mercury (mm Hg) versus time in minutes for the biasing members of FIG. 17A and FIG. 17B. The biasing member of FIG. 17A and FIG. 17B was tested in a first scenario without the plenum 604 and in a second scenario with the plenum 604. Table 06 illustrates the change in pressure over time.

TABLE 06
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 122.4 | | 73.1 | |
| 0.5 | 116.1 | 6.3 | 70.1 | 3 |
| 1 | 114.3 | 1.8 | 69.6 | 0.5 |
| 2 | 112.1 | 2.2 | 69.4 | 0.2 |
| 3 | 111.0 | 1.1 | 69.5 | −0.1 |
| 4 | 109.9 | 1.1 | 69.2 | 0.3 |
| 5 | 109.4 | 0.5 | 68.9 | 0.3 |
| 6 | 108.6 | 0.8 | 68.6 | 0.3 |
| 7 | 107.9 | 0.7 | 68.1 | 0.5 |
| 8 | 107.2 | 0.7 | 68.2 | −0.1 |
| 9 | 106.7 | 0.5 | 68.4 | −0.2 |
| 10 | 106.2 | 0.5 | 68.4 | 0 |
| 15 | 104.0 | 2.2 | 68.0 | 0.4 |
| 20 | 101.9 | 2.1 | 67.4 | 0.6 |
| 25 | 100.1 | 1.8 | 66.9 | 0.5 |

| | | |
|---|---|---|
| Avg. change | 1.59 | 0.44 |
| (mmHg) | | |

As illustrated in Table 06, the biasing member was able to generate a higher negative pressure without the 40 mL plenum. The biasing member tested without the plenum maintained the negative pressure within about 18% of the initially developed pressure. The biasing member testing with the plenum were able to maintain the negative pressure within about 8% of the initially developed pressure.

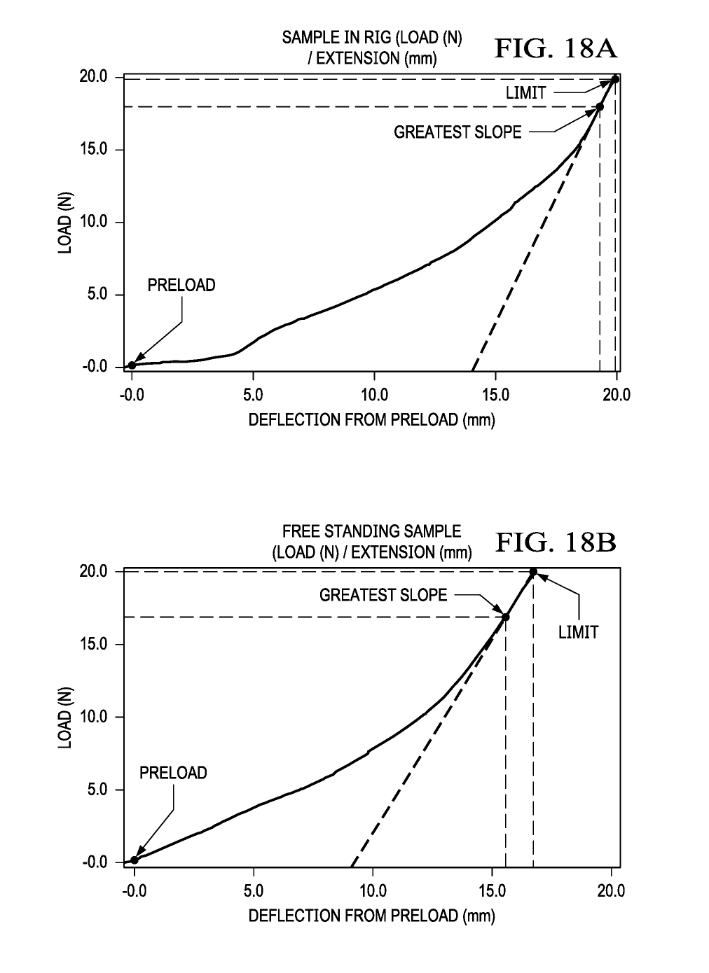

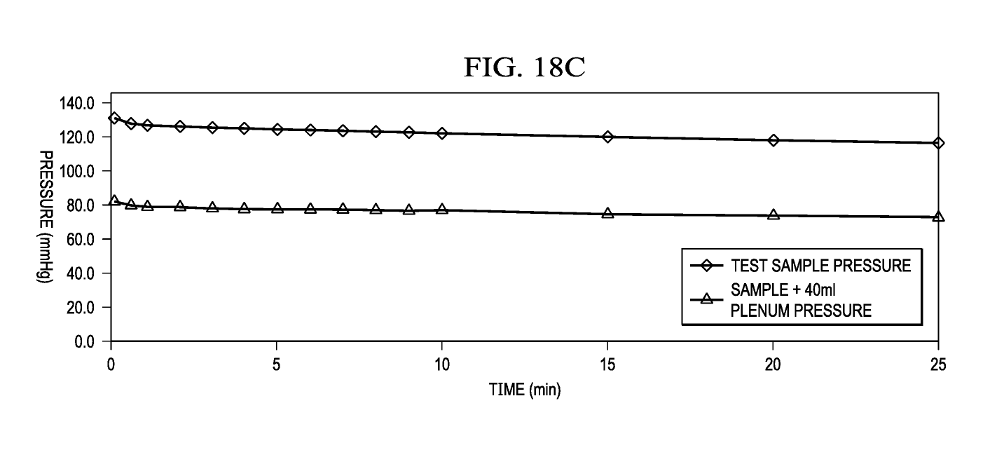

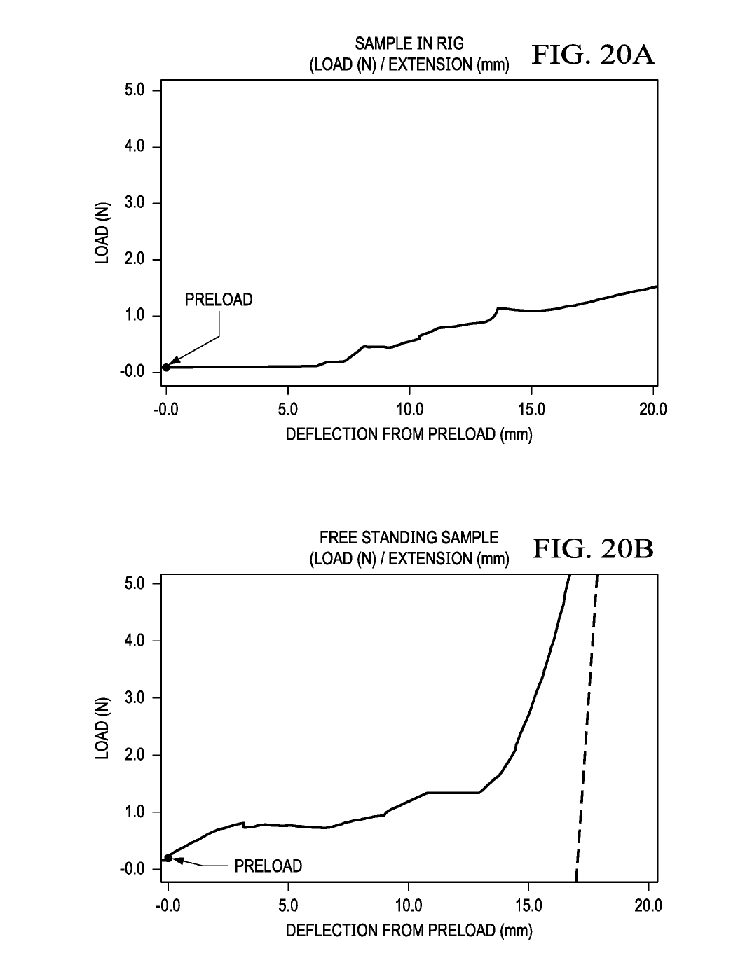

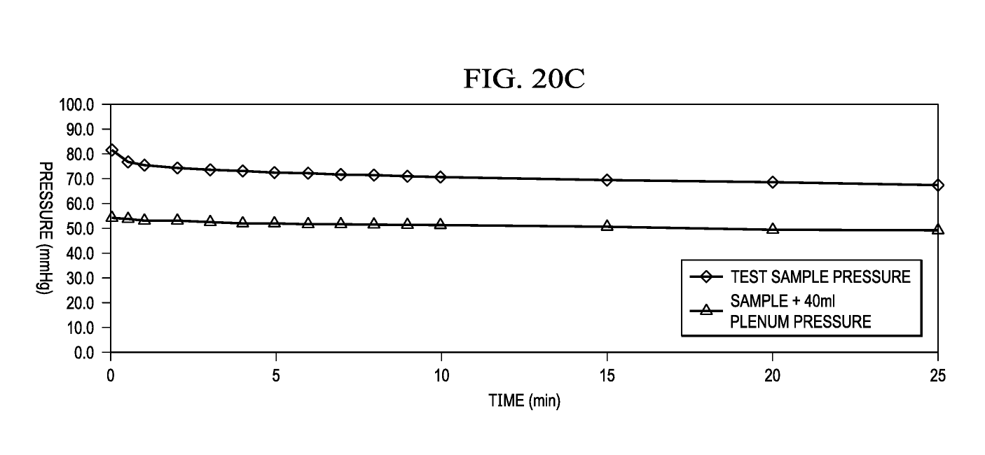

FIG. 18A is a line graph illustrating a load in Newtons (N) versus a deflection from preload in millimeters (mm) for the biasing member of FIG. 20A of the negative-pressure therapy system of FIG. 10. In particular, FIG. 18A illustrates a load in Newtons applied to the foam block 134 to produce the corresponding deflection of the foam block 134 from its preloaded position where the testing is conducted in the testing apparatus 600 of FIG. 14. FIG. 18B is a line graph illustrating a load in Newtons (N) versus a deflection from preload in millimeters (mm) for a free standing embodiment of the biasing member of FIG. 18A. In FIG. 18A and FIG. 18B, the foam block 134 was formed from a felted V.A.C.® GRANUFOAM™ dressing having a firmness factor of 5. The foam block 134 was cylindrical in shape having a diameter of about 40 mm, a height of about 30 mm, and a volume of about 37.7 cubic centimeters (“cc”). The foam block 134 was formed from a single layer. The preload was about 0.3 N. Table 07 illustrates the applied force to achieve the distance deflection.

TABLE 07

| Distance (mm) | Test Rig | Free Standing |
|---|---|---|
| 5 | 1.95 | 3.48 |
| 10 | 5.48 | 7.88 |
| 15 | 10.38 | 16.17 |
| 20 | — | — |
| 25 | — | — |
| 30 | — | — |
| Distance @ 20 N: | 19.90 | 16.40 |
| Average (N): | 5.94 | 9.18 |

As illustrated in Table 07, less load was used to compress the free standing sample to the same deflection as the sample in the rig.

FIG. 18C is a line graph illustrating a pressure in millimeters mercury (mm Hg) versus time in minutes for the biasing members of FIG. 18A and FIG. 18B. The biasing member of FIG. 18A and FIG. 18B was tested in a first scenario without the plenum 604 and in a second scenario with the plenum 604. Table 08 illustrates the change in pressure over time.

TABLE 08
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 131.0 | | 83.3 | |
| 0.5 | 127.8 | 3.2 | 80.3 | 3 |
| 1 | 127.1 | 0.7 | 79.9 | 0.4 |
| 2 | 126.3 | 0.8 | 79.5 | 0.4 |
| 3 | 125.7 | 0.6 | 79.2 | 0.3 |
| 4 | 125.2 | 0.5 | 78.9 | 0.3 |
| 5 | 124.9 | 0.3 | 78.7 | 0.2 |
| 6 | 124.4 | 0.5 | 78.5 | 0.2 |
| 7 | 124.0 | 0.4 | 78.4 | 0.1 |
| 8 | 123.8 | 0.2 | 78.2 | 0.2 |
| 9 | 123.5 | 0.3 | 78.0 | 0.2 |
| 10 | 123.1 | 0.4 | 78.0 | 0 |
| 15 | 121.8 | 1.3 | 77.4 | 0.6 |
| 20 | 120.6 | 1.2 | 76.7 | 0.7 |
| 25 | 119.4 | 1.2 | 76.0 | 0.7 |

| | | |
|---|---|---|
| Avg. change | 0.83 | 0.52 |
| (mmHg) | | |

As illustrated in Table 08, the biasing member was able to generate a higher negative pressure without the 40 mL plenum. The biasing member tested without the plenum maintained the negative pressure within about 9% of the initially developed pressure. The biasing member testing with the plenum were able to maintain the negative pressure within about 9% of the initially developed pressure.

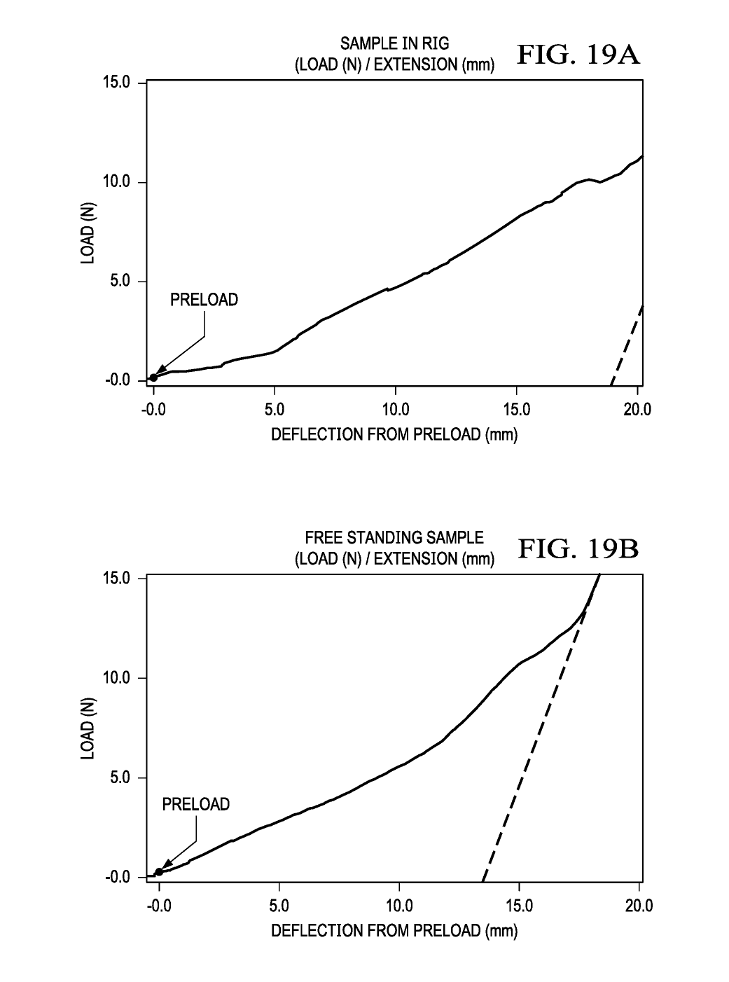

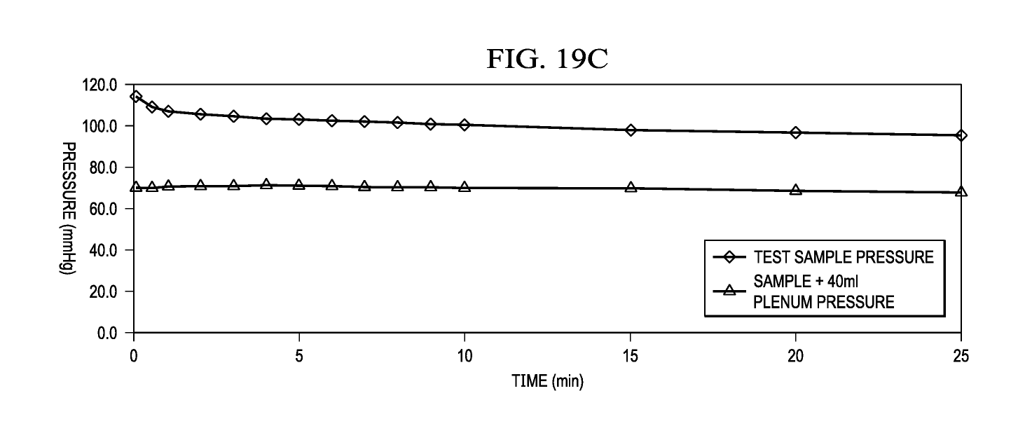

FIG. 19A is a line graph illustrating a load in Newtons (N) versus a deflection from preload in millimeters (mm) for the biasing member of FIG. 20B of the negative-pressure therapy system of FIG. 10. In particular, FIG. 19A illustrates a load in Newtons applied to the foam block 134 to produce the corresponding deflection of the foam block 134 from its preloaded position where the testing is conducted in the testing apparatus 600 of FIG. 14. FIG. 19B is a line graph illustrating a load in Newtons (N) versus a deflection from preload in millimeters (mm) for a free standing embodiment of the biasing member of FIG. 19A. In FIG. 19A and FIG. 19B, the foam block 134 was formed from a felted V.A.C.® GRANUFOAM™ dressing having a firmness factor of 5. The foam block 134 was cylindrical in shape having a diameter of about 40 mm, a height of about 30 mm, and a volume of about 37.7 cubic centimeters (“cc”). The foam block 134 was formed from a single layer. The foam block 134 included the plurality of holes having an average effective diameter of about 3 mm. The preload was about 0.3 N. Table 09 illustrates the applied force to achieve the distance deflection.

TABLE 09

| Distance (mm) | Test Rig | Free Standing |
|---|---|---|
| 5 | 1.48 | 2.90 |
| 10 | 4.78 | 5.56 |
| 15 | 8.35 | 10.79 |
| 20 | 11.10 | — |
| 25 | 18.62 | — |
| 30 | — | — |
| Distance @ 21 N: | 25.70 | 19.80 |
| Average (N): | 8.87 | 6.42 |

As illustrated in Table 09, less load was used to compress the free standing sample to the same deflection as the sample in the rig.

FIG. 19C is a line graph illustrating a pressure in millimeters mercury (mm Hg) versus time in minutes for the biasing members of FIG. 19A and FIG. 19B. The biasing member of FIG. 19A and FIG. 19B was tested in a first scenario without the plenum 604 and in a second scenario with the plenum 604. Table 10 illustrates the change in pressure over time.

TABLE 10
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 115.2 | | 71.4 | |
| 0.5 | 110.0 | 5.2 | 71.4 | 0 |
| 1 | 108.2 | 1.8 | 71.9 | −0.5 |
| 2 | 106.8 | 1.4 | 72.1 | −0.2 |
| 3 | 105.7 | 1.1 | 72.4 | −0.3 |
| 4 | 104.7 | 1 | 72.5 | −0.1 |
| 5 | 104.4 | 0.3 | 72.6 | −0.1 |
| 6 | 104.0 | 0.4 | 72.4 | 0.2 |
| 7 | 103.6 | 0.4 | 72.4 | 0 |
| 8 | 103.2 | 0.4 | 72.4 | 0 |
| 9 | 102.6 | 0.6 | 72.3 | 0.1 |
| 10 | 102.3 | 0.3 | 72.3 | 0 |
| 15 | 100.5 | 1.8 | 72.0 | 0.3 |
| 20 | 99.6 | 0.9 | 71.8 | 0.2 |
| 25 | 98.9 | 0.7 | 71.5 | 0.3 |

| | | |
|---|---|---|
| Avg. change | 1.16 | −0.01 |
| (mmHg) | | |

As illustrated in Table 10, the biasing member was able to generate a higher negative pressure without the 40 mL plenum. The biasing member tested without the plenum maintained the negative pressure within about 14% of the initially developed pressure. The biasing member testing with the plenum were able to maintain the negative pressure at about the initially developed pressure.

FIG. 20A is a line graph illustrating a load in Newtons (N) versus a deflection from preload in millimeters (mm) for the biasing member of FIG. 20C of the negative-pressure therapy system of FIG. 10. In particular, FIG. 20A illustrates a load in Newtons applied to the foam block 134 to produce the corresponding deflection of the foam block 134 from its preloaded position where the testing is conducted in the testing apparatus 600 of FIG. 14. FIG. 20B is a line graph illustrating a load in Newtons (N) versus a deflection from preload in millimeters (mm) for a free standing embodiment of the biasing member of FIG. 20A. In FIG. 20A and FIG. 20B, the foam block 134 was formed from a felted V.A.C.® GRANUFOAM™ dressing having a firmness factor of 5. The foam block 134 was cylindrical in shape having a diameter of about 40 mm, a height of about 30 mm, and a volume of about 37.7 cubic centimeters (“cc”). The foam block 134 was formed from a single layer. The foam block 134 included the plurality of holes having an average effective diameter of about 5 mm. The preload was about 0.3 N. Table 11 illustrates the applied force to achieve the distance deflection.

TABLE 11

| Distance (mm) | Test Rig | Free Standing |
|---|---|---|
| 5 | 0.10 | 0.75 |
| 10 | 0.57 | 1.23 |
| 15 | 1.07 | 2.74 |
| 20 | 1.42 | 19.13 |
| 25 | 2.30 | — |
| 30 | 7.34 | — |
| Distance @ 22 N: | 34.10 | 20.20 |
| Average (N): | 6.70 | 5.96 |

As illustrated in Table 11, less load was used to compress the free standing sample to the same deflection as the sample in the rig.

FIG. 20C is a line graph illustrating a pressure in millimeters mercury (mm Hg) versus time in minutes for the biasing members of FIG. 20A and FIG. 20B. The biasing member of FIG. 20A and FIG. 20B was tested in a first scenario without the plenum 604 and in a second scenario with the plenum 604. Table 12 illustrates the change in pressure over time.

TABLE 12
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 82.1 | | 55.3 | |
| 0.5 | 77.4 | 4.7 | 54.6 | 0.7 |
| 1 | 76.1 | 1.3 | 54.0 | 0.6 |
| 2 | 74.9 | 1.2 | 53.7 | 0.3 |
| 3 | 74.2 | 0.7 | 53.6 | 0.1 |
| 4 | 73.8 | 0.4 | 53.2 | 0.4 |
| 5 | 73.2 | 0.6 | 53.0 | 0.2 |
| 6 | 72.9 | 0.3 | 53.0 | 0 |
| 7 | 72.5 | 0.4 | 52.9 | 0.1 |
| 8 | 72.3 | 0.2 | 53.0 | −0.1 |
| 9 | 72.0 | 0.3 | 52.9 | 0.1 |
| 10 | 72.0 | 0 | 52.7 | 0.2 |
| 15 | 70.9 | 1.1 | 52.4 | 0.3 |
| 20 | 70.3 | 0.6 | 51.6 | 0.8 |
| 25 | 69.7 | 0.6 | 51.6 | 0 |

| | | |
|---|---|---|
| Avg. change | 0.89 | 0.26 |
| (mmHg) | | |

As illustrated in Table 12, the biasing member was able to generate a higher negative pressure without the 40 mL plenum. The biasing member tested without the plenum maintained the negative pressure within about 15% of the initially developed pressure. The biasing member testing with the plenum were able to maintain the negative pressure within about 7% of the initially developed pressure.

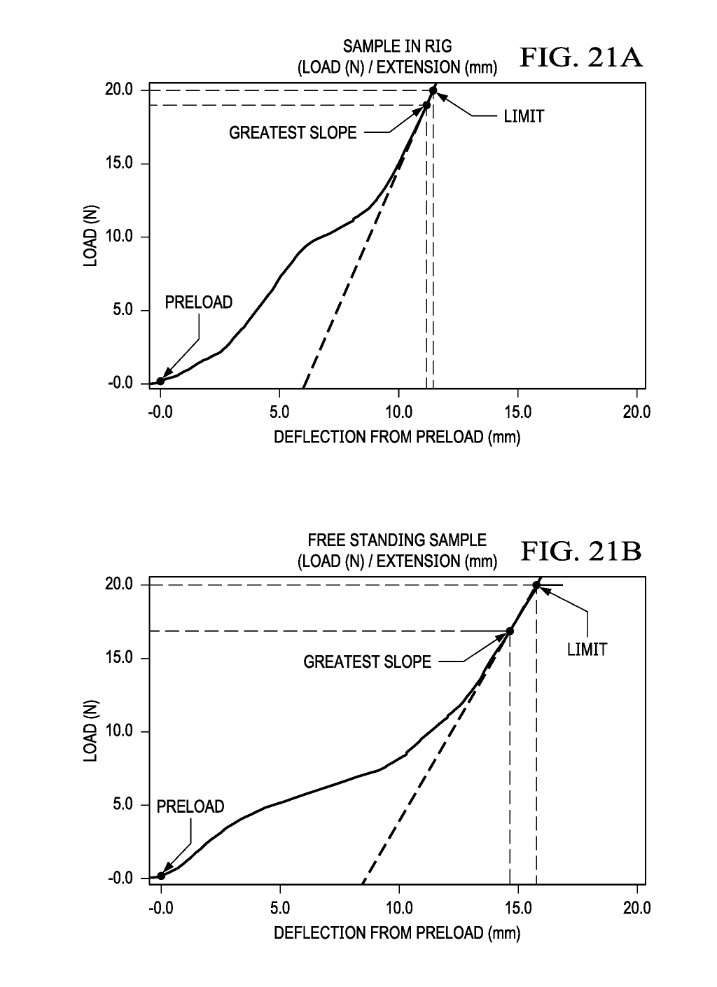

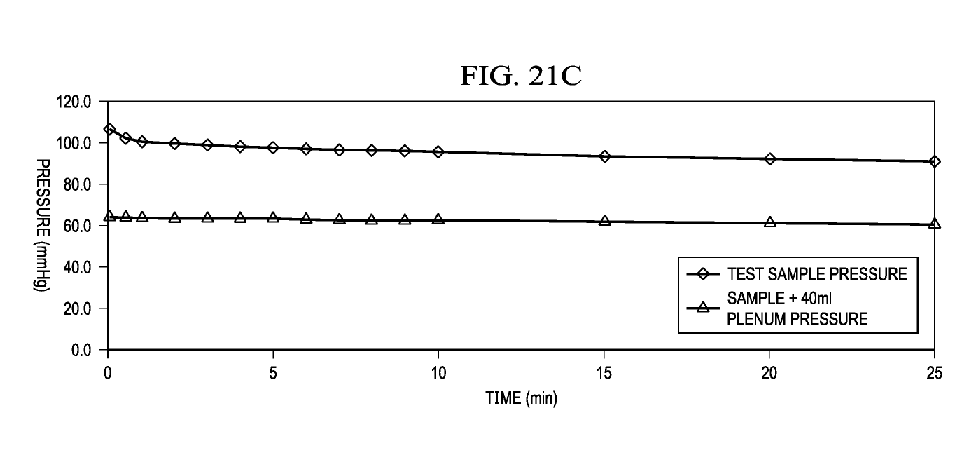

FIG. 21A is a line graph illustrating a load in Newtons (N) versus a deflection from preload in millimeters (mm) for the biasing member of the negative-pressure therapy system of FIG. 10. In particular, FIG. 21A illustrates a load in Newtons applied to the foam block 134 to produce the corresponding deflection of the foam block 134 from its preloaded position where the testing is conducted in the testing apparatus 600 of FIG. 14. FIG. 21B is a line graph illustrating a load in Newtons (N) versus a deflection from preload in millimeters (mm) for a free standing embodiment of the biasing member of FIG. 21A. In FIG. 21A and FIG. 21B, the foam block 134 was formed from a partially felted foam, for example, a rolled felted foam or a polyurethane foam having high density regions and low density regions. In some embodiments, the foam block 134 had about 45 ppi and regions having a firmness factor of 2 adjacent regions having a firmness factor of about 3 and a pitch of about 10 mm. The foam block 134 was cylindrical in shape having a diameter of about 40 mm, a height of about 30 mm, and a volume of about 37.7 cubic centimeters (“cc”). The foam block 134 was formed from a single layer. The preload was about 0.3 N. Table 13 illustrates the applied force to achieve the distance deflection.

TABLE 13

| Distance (mm) | Test Rig | Free Standing |
|---|---|---|
| 5 | 7.25 | 5.20 |
| 10 | 15.12 | 8.20 |
| 15 | — | 18.00 |
| 20 | — | — |
| 25 | — | — |
| 30 | — | — |
| Distance @ 23 N: | 11.40 | 15.70 |
| Average (N): | 11.19 | 10.47 |

As illustrated in Table 13, less load was used to compress the free standing sample to the same deflection as the sample in the rig.

FIG. 21C is a line graph illustrating a pressure in millimeters mercury (mm Hg) versus time in minutes for the biasing members of FIG. 21A and FIG. 21B. The biasing member of FIG. 21A and FIG. 21B was tested in a first scenario without the plenum 604 and in a second scenario with the plenum 604. Table 14 illustrates the change in pressure over time.

TABLE 14
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 108.0 | | 66.0 | |
| 0.5 | 103.7 | 4.3 | 65.4 | 0.6 |
| 1 | 101.9 | 1.8 | 65.1 | 0.3 |
| 2 | 101.1 | 0.8 | 64.9 | 0.2 |
| 3 | 100.2 | 0.9 | 64.9 | 0 |
| 4 | 99.5 | 0.7 | 64.9 | 0 |
| 5 | 99.1 | 0.4 | 65.1 | −0.2 |
| 6 | 98.6 | 0.5 | 65.0 | 0.1 |
| 7 | 98.1 | 0.5 | 64.8 | 0.2 |
| 8 | 98.0 | 0.1 | 64.5 | 0.3 |
| 9 | 97.8 | 0.2 | 64.2 | 0.3 |
| 10 | 97.5 | 0.3 | 64.5 | −0.3 |
| 15 | 95.7 | 1.8 | 64.8 | −0.3 |
| 20 | 94.9 | 0.8 | 64.2 | 0.6 |
| 25 | 94.0 | 0.9 | 63.8 | 0.4 |

| | | |
|---|---|---|
| Avg. change | 1.00 | 0.16 |
| (mmHg) | | |

As illustrated in Table 14, the biasing member was able to generate a higher negative pressure without the 40 mL plenum. The biasing member tested without the plenum maintained the negative pressure within about 13% of the initially developed pressure. The biasing member testing with the plenum were able to maintain the negative pressure within about 3% of the initially developed pressure.

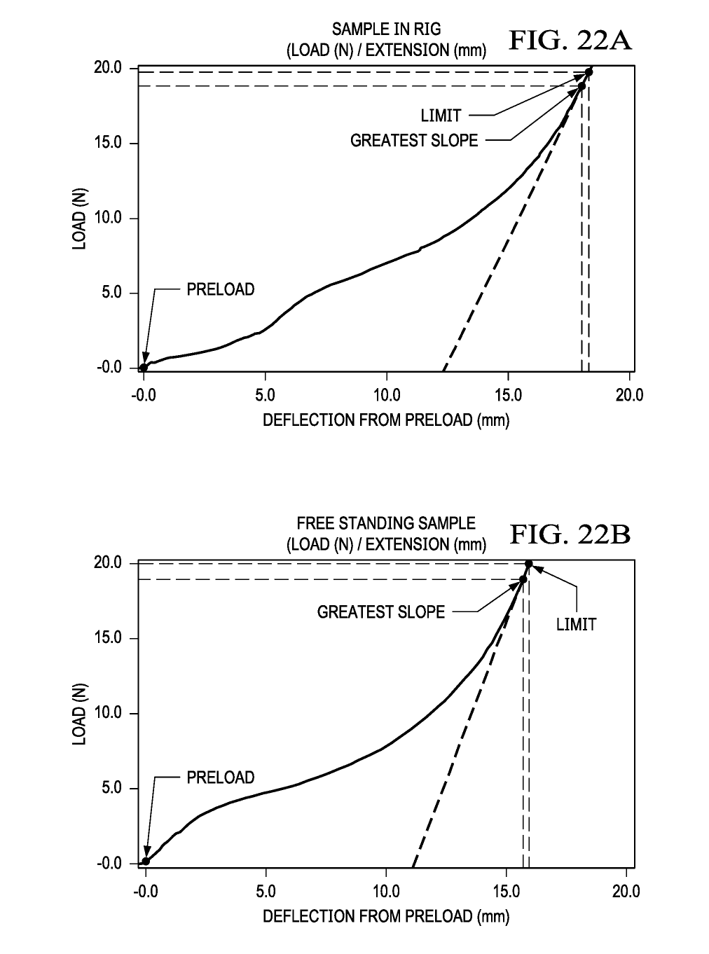

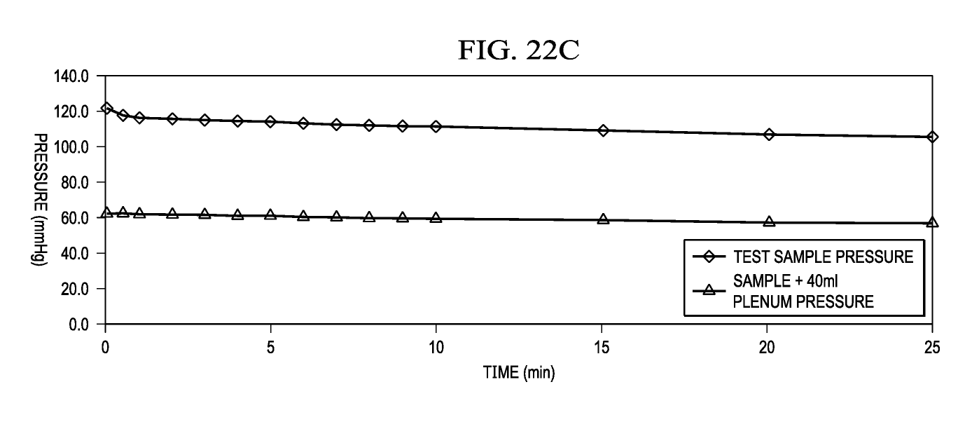

FIG. 22A is a line graph illustrating a load in Newtons (N) versus a deflection from preload in millimeters (mm) for the biasing member of the negative-pressure therapy system of FIG. 10. In particular, FIG. 22A illustrates a load in Newtons applied to the foam block 134 to produce the corresponding deflection of the foam block 134 from its preloaded position where the testing is conducted in the testing apparatus 600 of FIG. 14. FIG. 22B is a line graph illustrating a load in Newtons (N) versus a deflection from preload in millimeters (mm) for a free standing embodiment of the biasing member of FIG. 22A. In FIG. 22A and FIG. 22B, the foam block 134 was formed from a felted V.A.C.® GRANUFOAM™ dressing having a firmness factor of 5. The foam block 134 was cylindrical in shape having a diameter of about 40 mm, a height of about 30 mm, and a volume of about 37.7 cubic centimeters (“cc”). The foam block 134 was formed from a single layer. The foam block 134 may be similar to the foam block of FIG. 20B, the felting of the foam block 134 oriented perpendicular to the direction of deflection and having the plurality of holes 135 having an average effective diameter of about 3 mm. The preload was about 0.3 N. Table 15 illustrates the applied force to achieve the distance deflection.

TABLE 15

| Distance (mm) | Test Rig | Free Standing |
|---|---|---|
| 5 | 2.62 | 4.80 |
| 10 | 7.14 | 7.83 |
| 15 | 12.28 | 16.53 |
| 20 | — | — |
| 25 | — | — |
| 30 | — | — |
| Distance @ 24 N: | 18.20 | 15.58 |
| Average (N): | 7.35 | 9.72 |

As illustrated in Table 15, less load was used to compress the free standing sample to the same deflection as the sample in the rig.

FIG. 22C is a line graph illustrating a pressure in millimeters mercury (mm Hg) versus time in minutes for the biasing members of FIG. 22A and FIG. 22B. The biasing member of FIG. 22A and FIG. 22B was tested in a first scenario without the plenum 604 and in a second scenario with the plenum 604. Table 16 illustrates the change in pressure over time.

TABLE 16
Time (min)/Pressure (mmHg)

| | |
|---|---|
| | Sample + 40 ml |
| Test sample | plenum |

| Time | Pressure | Change | Pressure | Change |
|---|---|---|---|---|
| 0 | 121.8 | | 62.7 | |
| 0.5 | 117.9 | 3.9 | 62.7 | 0 |
| 1 | 116.7 | 1.2 | 62.6 | 0.1 |
| 2 | 115.9 | 0.8 | 62.4 | 0.2 |
| 3 | 115.4 | 0.5 | 62.2 | 0.2 |
| 4 | 114.9 | 0.5 | 61.8 | 0.4 |
| 5 | 114.5 | 0.4 | 61.7 | 0.1 |
| 6 | 113.8 | 0.7 | 61.3 | 0.4 |
| 7 | 113.2 | 0.6 | 61.2 | 0.1 |
| 8 | 112.8 | 0.4 | 60.9 | 0.3 |
| 9 | 112.5 | 0.3 | 60.9 | 0 |
| 10 | 112.4 | 0.1 | 60.8 | 0.1 |
| 15 | 110.8 | 1.6 | 60.7 | 0.1 |
| 20 | 109.1 | 1.7 | 59.9 | 0.8 |
| 25 | 108.2 | 0.9 | 59.4 | 0.5 |

| | | |
|---|---|---|
| Avg. change | 0.97 | 0.24 |
| (mmHg) | | |

As illustrated in Table 16, the biasing member was able to generate a higher negative pressure without the 40 mL plenum. The biasing member tested without the plenum maintained the negative pressure within about 11% of the initially developed pressure. The biasing member testing with the plenum were able to maintain the negative pressure within about 5% of the initially developed pressure.

As described with respect to FIGS. 17-22C and Tables 1-16, various biasing members were tested to determine a range of suitable materials for the foam block 134. Based on the collected data, the tested materials were compared. Each material was tested free standing and in the testing apparatus 600 to determine an average force to compress the material. A lower average force to compress a material a greater distance may be preferred. The pressure generated during testing of each material also can be viewed in light of the average force to compress the material. Materials capable of producing a highest pressure with an application of the least force may be preferred. In view of the testing, an exemplary foam block 134 formed from V.A.C.® GRANUFOAM™ dressing having a firmness factor of 5 and the holes 135 having an average effective diameter of about 3 mm was selected.

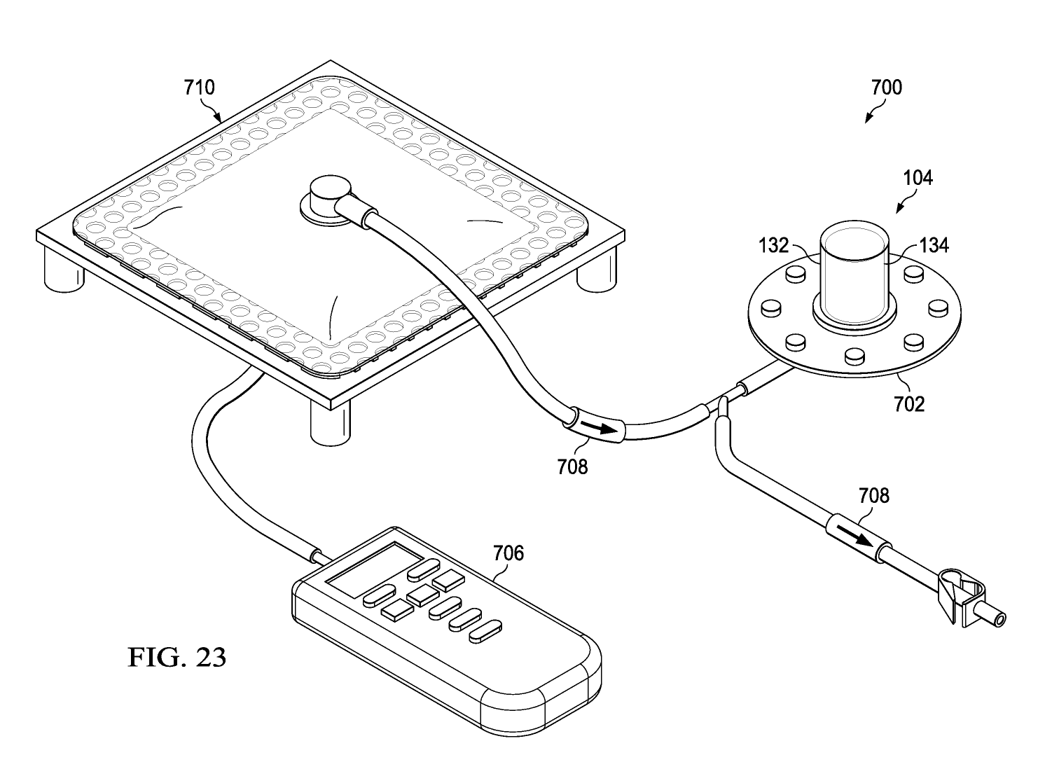

FIG. 23 is a perspective view illustrating additional details of a testing apparatus 700 that may be associated with some embodiments of the negative-pressure therapy system. The testing apparatus 700 can include a receiver 702, a pressure tester 706, a valve 708, and a dressing 710. Each of the components can be fluidly coupled by one or more conduits or other fluid connectors. The receiver 702 can be a device configured to provide a re-sealable source chamber for testing of the biasing member. For example, the receiver 702 can be a pair of separable plates configured to receive the film layer 132 and the foam block 134 and seal around the source flange 136 to form the source cavity 138. The receiver 702 can position the foam block 134 and the film layer 132 so that the foam block 134 and the film layer 132 can be compressed by hand. The receiver 702 can also provide a mechanism to fluidly couple the source cavity 138 to other components. The pressure tester 706 can be a manometer capable of measuring pressure in a system, testing for leaks, and measuring flow within a system. In some embodiments, the pressure tester 706 can be a 2022 Pressure Tester rated for 0 to 1500 mm Hg produced by Sifam Instruments. The dressing 710 can be a dressing for providing reduced pressure and fluid absorption at a tissue site. For example, the dressing 710 may have a pouch, such as the pouch 105, and a cover, such as the cover 103. In some embodiments, the dressing 710 may be a NANOVA™ dressing.

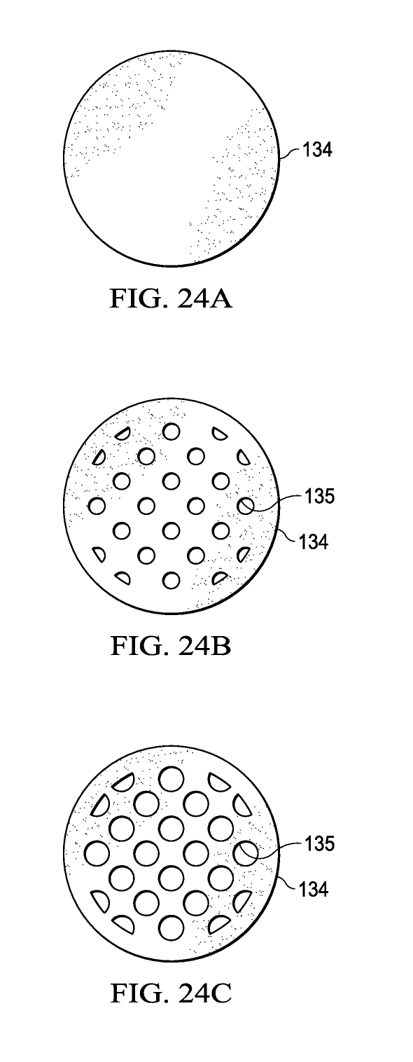

The foam block 134 of the negative-pressure source 104 can be formed from a plurality of materials including felted foam. To determine some characteristics of embodiments of a biasing member, some example embodiments of the foam block 134 were observed in the testing apparatus 700 and data was recorded. In particular, three different variations of a biasing member were tested to determine the load required to compress the biasing member to predetermined levels of deflection. FIG. 24A is a top view illustrating additional details of a first biasing member for which characteristics were observed in the testing apparatus 700. In some embodiments, the foam block 134 can be a solid foam construction having no holes. FIG. 24B is a top view illustrating additional details of a second biasing member for which characteristics were observed in the testing apparatus 700. In some embodiments, the foam block 134 can have a plurality of holes 135. Each of the plurality of holes 135 may extend through the foam block 134 from a first surface to a second surface. In some embodiments, the plurality of holes 135 can each have an average effective diameter of about 3 mm. The plurality of holes 135 can be equidistantly spaced from each other. FIG. 24C is a top view illustrating additional details of a third biasing member for which characteristics were observed in the testing apparatus 700. In some embodiments, the foam block 134 can have the plurality of holes 135. Each of the plurality of holes 135 may extend through the foam block 134 from a first surface to a second surface. In some embodiments, the plurality of holes 135 can each have an average effective diameter of about 5 mm. The plurality of holes 135 can be equidistantly spaced from each other. In the foam block 134 of each of FIG. 24A, FIG. 24B, and FIG. 24C, the foam block 134 may be formed from a felted foam having a firmness factor of 5 and may have a diameter of about 40 mm.

During testing, observations indicated that the foam block 134 having the holes 135 with 3 mm average effective diameter may be easier to compress if pressed from the sides rather than the top of the foam block 134. Compression of the foam block 134 from the sides compresses the foam block 134 perpendicular to the direction of the holes 135; the foam block 134 is compressed horizontally. Compression of the foam block 134 parallel to the direction of the holes 135 can be referred to as vertical compression. It was speculated that due to the construction of the felted foam the elastic modulus could be different for different axes of compression making compression easier if the sample had been cut on its side. In another testing iteration, a foam block 134 having the holes 135 was formed from a 30 mm cube. The iteration was used to determine if having two parts and a drape material effect test results. In all tests using either the 30 mm cube or the 40 mm foam blocks 134, the foam blocks 134 compressed vertically required a lower compression force than the foam blocks 134 compressed horizontally. However, in pressure tests the horizontally compressed foam blocks 134 were able generated higher negative pressures, for example, 121.8 mmHg using horizontal compression versus 115.2 mmHg using vertical compression. In further testing, felted foam blocks 134 having a 3 mm average effective diameter of the holes 135 were determined to provide higher negative pressure while also being easier to compress than felted foam blocks 134 having no holes 135.

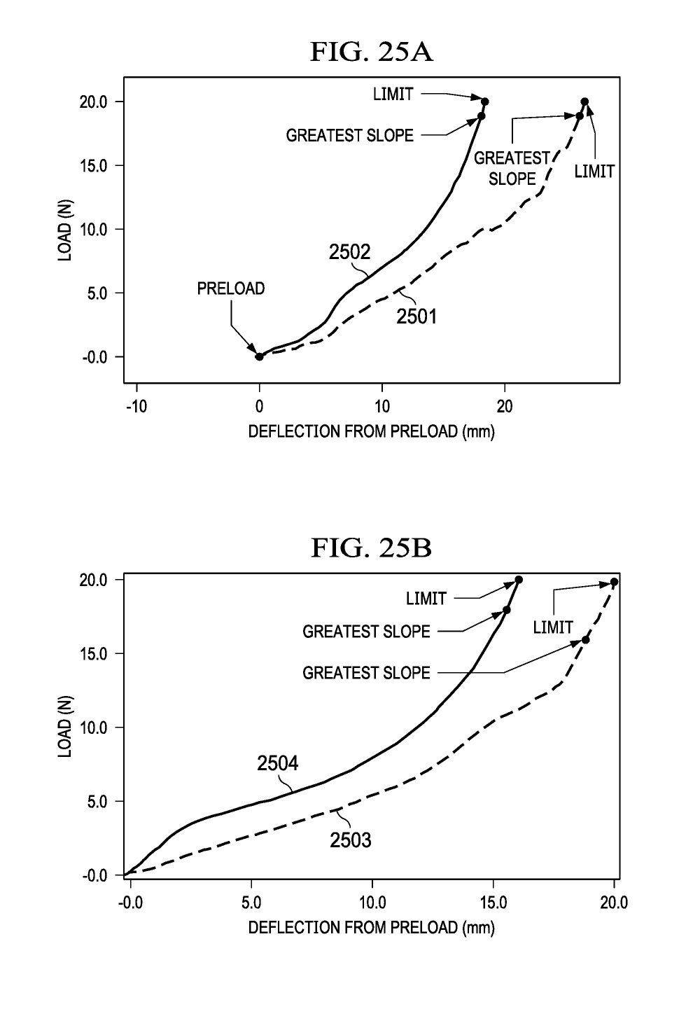

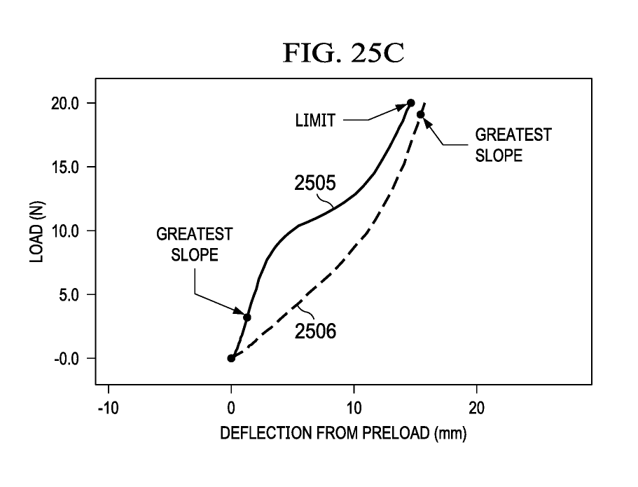

FIG. 25A is a line graph illustrating a load in Newtons (N) versus a deflection from preload in millimeters (mm) for a biasing member of the negative-pressure therapy system of FIG. 10. In particular, FIG. 25A illustrates a load in Newtons applied to the foam block 134 to produce the corresponding deflection of the foam block 134 from its preloaded position where the testing is conducted in the testing apparatus 700 of FIG. 23. The foam block 134 was cylindrical in shape having a diameter of about 40 mm, a height of about 30 mm, and a volume of about 37.7 cubic centimeters (“cc”). The foam block 134 was formed from a single layer of felted foam having 45 ppi and a firmness factor of 5. The foam block 134 included the plurality of holes 135 having an average effective diameter of about 3 mm as illustrated in FIG. 24B. In a testing process, the foam block 134 was oriented in the testing apparatus so that the direction of felting of the foam block 134 was parallel to the direction of application of the load to the foam block 134, vertical compression, and the foam black 134 was enclosed by the film layer 132. Line 2501 illustrates the change in deflection with respect to the increasing application of the load to the foam block 134. In another testing process, the foam block 134 was oriented in the testing apparatus so that the direction of felting of the foam block 134 was perpendicular to the direction of application of the load to the foam block 134, horizontal compression. Line 2502 illustrates the change in deflection with respect to the increasing application of the load to the foam block 134. Table 17 illustrates the applied force to achieve the distance deflection.

TABLE 17
Deflection (mm)/Load (N)

| | |
|---|---|
| Line 2501 | Line 2502 |

| Deflection | Load | | Load | |
|---|---|---|---|---|
| (mm) | (N) | Change | (N) | Change |
| 2.5 | 0.67 | | 1.17 | |
| 5 | 1.53 | 0.86 | 2.62 | 1.45 |
| 7.5 | 3.34 | 1.81 | 5.52 | 2.90 |
| 10 | 4.70 | 1.36 | 7.06 | 1.54 |
| 12.5 | 6.37 | 1.67 | 9.07 | 2.01 |
| 15 | 8.27 | 1.90 | 12.27 | 3.20 |
| 17.5 | 10.03 | 1.76 | 17.48 | 5.21 |
| 20 | 11.15 | 1.12 | — | N/A |

| | | |
|---|---|---|
| Limit (20 N): | 25.8 mm | 18.34 mm |

As illustrated in Table 17, an increase in the application of force increased the amount the biasing member was deflected. Line 2501 and line 2502 illustrate that less load was necessary to deflect the foam block 134 an equal amount where the load was applied vertically.

FIG. 25B is a line graph illustrating a load in Newtons (N) versus a deflection from preload in millimeters (mm) for a free standing embodiment of the biasing member of FIG. 25A. In particular, FIG. 25B illustrates a load in Newtons applied to the foam block 134 to produce the corresponding deflection of the foam block 134 from its preloaded position where the testing is conducted in a tensile test machine independent of the testing apparatus 700 of FIG. 23. The foam block 134 was cylindrical in shape having a diameter of about 40 mm, a height of about 30 mm, and a volume of about 37.7 cubic centimeters (“cc”). The foam block 134 was formed from a single layer of felted foam having 45 ppi and a firmness factor of 5. The foam block 134 included the plurality of holes having an average effective diameter of about 3 mm as illustrated in FIG. 24B. In a testing process, the foam block 134 was oriented in the testing apparatus 700 so that the direction of felting of the foam block 134 was parallel to the direction of application of the load to the foam block 134, vertical compression, and the foam block 134 was not enclosed by the film layer 132. Line 2503 illustrates the change in deflection with respect to the increasing application of the load to the foam block 134. In another testing process, the foam block 134 was oriented in the testing apparatus 700 so that the direction of felting of the foam block 134 was perpendicular to the direction of application of the load to the foam block 134, horizontal compression. Line 2504 illustrates the change in deflection with respect to the increasing application of the load to the foam block 134. Table 18 illustrates the applied force to achieve the distance deflection.

TABLE 18
Deflection (mm)/Load (N)

| | |
|---|---|
| Line 2503 | Line 2504 |

| Deflection | Load | | Load | |
|---|---|---|---|---|
| (mm) | (N) | Change | (N) | Change |
| 2.5 | 1.51 | | 3.48 | |
| 5 | 2.75 | 1.24 | 4.74 | 1.26 |
| 7.5 | 4.07 | 1.32 | 6.12 | 1.38 |
| 10 | 5.57 | 1.50 | 8.12 | 2.00 |
| 12.5 | 7.50 | 1.93 | 11.24 | 3.12 |
| 15 | 10.68 | 1.93 | 16.66 | 5.42 |
| 17.5 | 13.13 | 3.18 | — | N/A |
| 20 | — | N/A | — | N/A |

| | | |
|---|---|---|
| Limit (20 N): | 19.80 mm | 15.92 mm |

As illustrated in Table 18, an increase in the application of force increased the amount the biasing member was deflected. Line 2503 and line 2504 illustrate that less load was necessary to deflect the foam block 134 an equal amount where the load was applied vertically.

FIG. 25C is a line graph illustrating a load in Newtons (N) versus a deflection from preload in millimeters (mm) for a biasing member of the negative-pressure therapy system of FIG. 10. In particular, FIG. 25C illustrates a load in Newtons applied to the foam block 134 to produce the corresponding deflection of the foam block 134 from its preloaded position where the testing is conducted independently of the testing apparatus 700 of FIG. 23. The foam block 134 was an unperforated 30 mm cube formed from a single layer of felted foam having 45 ppi and a firmness factor of 5. In a testing process, the foam block 134 was oriented so that the direction of felting of the foam block 134 was parallel to the direction of application of the load to the foam block 134, vertical compression, and the foam block 134 was enclosed by the film layer 132. Line 2505 illustrates the change in deflection with respect to the increasing application of the load to the foam block 134. In another testing process, the foam block 134 was oriented so that the direction of felting of the foam block 134 was perpendicular to the direction of application of the load to the foam block 134, horizontal compression. Line 2506 illustrates the change in deflection with respect to the increasing application of the load to the foam block 134. Table 19 illustrates the applied force to achieve the distance deflection.

TABLE 19
Deflection (mm)/Load (N)

| | |
|---|---|
| Line 2505 | Line 2506 |

| Deflection | Load | | Load | |
|---|---|---|---|---|
| (mm) | (N) | Change | (N) | Change |
| 2.5 | 2.03 | | 7.05 | |
| 5 | 3.93 | 1.90 | 10.02 | 2.97 |
| 7.5 | 6.10 | 2.17 | 11.14 | 1.12 |
| 10 | 8.65 | 2.55 | 12.81 | 1.67 |
| 12.5 | 12.11 | 3.46 | 15.81 | 3.00 |
| 15 | 17.70 | 5.59 | — | N/A |
| 17.5 | — | N/A | — | N/A |
| 20 | — | N/A | — | N/A |

| | | |
|---|---|---|
| Limit (20 N): | 15.75 mm | 14.61 mm |

As illustrated in Table 19, an increase in the application of force increased the amount the biasing member was deflected. Line 2505 and line 2506 illustrate that less load was necessary to deflect the foam block 134 an equal amount where the load was applied vertically. Based on the data from Table 17, Table 18, and Table 19, a determination was made that the foam block 134 having the holes 135 of about 3 mm each and oriented for vertical compression required less force to compress the foam block 134.

Table 20 illustrates the maximum attainable negative pressure within the dressing 710 of the testing apparatus 700 of FIG. 23. Specifically, various valve arrangements were used to collect data regarding the appropriate valves and number of valves for the system of FIG. 10. The biasing member used in the testing apparatus 700 was the foam block 134 formed from a single layer of felted foam having 45 ppi and a firmness factor of 5. The foam block 134 included the holes 135 having an average effective diameter of about 3 mm. Variations in maximum attainable negative pressure were observed in response to use of different valve arrangements and different valve types. For the test using two one-way valves, a first one-way valve was positioned to exhaust fluid from the receiver 702 to the ambient environment, and a second one-way valve was positioned to exhaust fluid from the dressing 710 to the receiver 702. For the test where the valve 708 is a single one-way valve, the valve 708 was positioned to exhaust fluid from the receiver 702. The exemplary one-way valves were West Group FL check valve model 500 3″ H₂O spring, with a cracking pressure of 6 mbar to about 9 mbar were used. For the testing using a restrictor valve, the restrictor valve was positioned between the receiver 702 and the ambient environment. For example, the restrictor valve could be placed in the location of the valve 608 of FIG. 14. Referring to FIG. 23, tests conducted in the testing apparatus 700 included having one or two non-return valves and a determination of the suitability of a restrictor valve to replace the non-return valves. To determine the pressure in the dressing 710, pressure readings were taken from a port under the dressing 710. In each test, the foam block 134 was positioned in the receiver 702 and subsequently repeatedly compressed to draw fluid from the dressing 710 until compressions appeared to have no effect or were too difficult to perform, that is the CFD of the foam block 134 was unable to overcome the force exerted by the system. Each compression was 5 seconds from a previous compression to permit the foam block 134 to inflate. Each 5 second time period began at the conclusion of the previous compression. In some embodiments, compression became increasingly difficult after 5-7 compressions, and the foam block 134 was unable to overcome the force exerted by the developed negative pressure, although subsequent compressions greatly increased the pressure. Where the non-return valves were replaced with a restrictor valve, inflation time for the foam block 134 increased for each iteration of the foam block 134.

TABLE 20
Maximum attainable pressure within the dressing (mmHg)

| Number | | | | | | | | | | | |
|---|---|---|---|---|---|---|---|---|---|---|---|
| of | Two 1-way valves | | | | | Single 1-way valve | | | Restrictor valve | | |
| pumps | Test 1 | Test 2 | Test 3 | Test 4 | Avg. | Test 1 | Test 2 | Avg. | Test 1 | Test 2 | Avg. |
| Pump 1 | 14.7 | 5.7 | 13.1 | 6.3 | 10.0 | 12.9 | 2.4 | 7.7 | 5.6 | 4.4 | 5.0 |
| Pump 2 | 21.1 | 16.5 | 22.0 | 16.9 | 19.1 | 18.6 | 6.6 | 12.6 | 15.9 | 12.2 | 14.1 |
| Pump 3 | 30.3 | 23.5 | 36.8 | 23.2 | 28.5 | 22.2 | 15.2 | 18.7 | 24.6 | 20.7 | 22.7 |
| Pump 4 | 48.7 | 30.6 | 53.4 | 34.3 | 41.8 | 27.8 | 23.4 | 25.6 | 28.1 | 22.9 | 25.5 |
| Pump 5 | 63.3 | 47.5 | 71.8 | 50.6 | 58.3 | 30.4 | 26.6 | 28.5 | 30.8 | 25.5 | 28.2 |
| Pump 6 | 76.8 | 64.1 | 87.5 | 67.9 | 74.1 | 37.2 | 28.2 | 32.7 | 33.5 | 30.8 | 32.2 |
| Pump 7 | 91.6 | 79.2 | 100.2 | 81.4 | 88.1 | 38.1 | 32.4 | 35.3 | 36.0 | 35.9 | 36.0 |
| Pump 8 | 101.0 | 94.0 | 106.7 | 93.6 | 98.8 | 36.8 | 36.0 | 36.4 | 35.0 | 42.9 | 39.0 |
| Pump 9 | 108.6 | 106.1 | 113.6 | 101.4 | 107.4 | 38.7 | 41.3 | 40.0 | 40.2 | 44.7 | 42.5 |
| Pump 10 | 117.8 | 111.9 | 121.9 | 110.1 | 115.4 | 38.0 | 42.1 | 40.1 | 40.1 | 42.8 | 41.5 |
| Pump 11 | 124.0 | 119.5 | 125.0 | 118.5 | 121.8 | | 40.7 | 40.7 | 41.4 | 42.2 | 41.8 |
| Pump 12 | 126.3 | 128.4 | 130.3 | 125.7 | 127.7 | | 39.9 | 39.9 | 44.1 | 37.2 | 40.7 |
| Pump 13 | | 135.8 | | | 135.8 | | 39.0 | 39.0 | 46.8 | 35.1 | 41.0 |
| Pump 14 | | 134.6 | | | 134.6 | | | | 42.8 | 41.3 | 42.1 |
| Pump 15 | | | | | | | | | 46.3 | 41.5 | 43.9 |
| Pump 16 | | | | | | | | | 48.7 | 35.6 | 42.2 |
| Pump 17 | | | | | | | | | 48.0 | 37.5 | 42.8 |
| Pump 18 | | | | | | | | | 41.7 | 35.2 | 38.5 |
| Pump 19 | | | | | | | | | 42.9 | 33.5 | 38.2 |
| Pump 20 | | | | | | | | | 39.2 | 38.7 | 39.0 |

As illustrated in Table 20, the biasing member was compressed twenty total times, and the resulting pressure under the dressing 710 was measured after each pump. The observed data indicated that using two one-way valves in the testing apparatus 700 permitted the development of higher negative pressures at the dressing 710. The average negative pressures measured at the dressing 710 using the single one-way valve and the restrictor were about the same and less than the negative pressure measured at the dressing 710 using two one-way valves. The testing apparatus 700 permitted the foam block 134 of FIG. 24B to develop a negative-pressure of up to 134.6 mmHg inside the dressing 710. The negative-pressure was maintained above 50 mmHg for at least 40 minutes. Using two non-return valves allowed the testing apparatus 700 to almost triple the maximum internal negative pressure (135 mmHg v. 48.7 mmHg) over iterations where the testing apparatus 700 was configured with a single non-return valve or a restrictor valve. To increase the negative pressure another 50-60 mmHg considerably more effort was required to compress the foam block 134, that is, the CFD of the foam block 134 may be insufficient to cause expansion of the source cavity 138. Each further compression removed a small amount of fluid.

The systems, apparatuses, and methods described herein may provide significant advantages. For example, the dressing assemblies described herein may be soft and pliable having no rigid parts while capable of providing negative-pressure therapy. The dressing assemblies may be low-profile and provide a visual pressure indicator of the status of the dressing assembly. The dressing assemblies allow for the application of negative-pressure therapy to less acute tissue sites, while being low cost, low complexity, and having an improved exudate management. The dressing assemblies can have improved manufacturability and functionality by creating a vertical assembly construction with a reduced number of components that form each assembly. Functionality can be improved by having a reduced footprint for the negative-pressure source, thereby increasing the surface area available for patient therapy, providing improved conformability, and decreasing the risk of blocking an air pathway during operation.

While shown in a few illustrative embodiments, a person having ordinary skill in the art will recognized that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A system for providing negative-pressure therapy to a tissue site, the system comprising:
an absorbent;
a sealing layer configured to cover the absorbent;
a blister fluidly coupled to the absorbent and having a collapsed position and an expanded position, the blister comprising:
a base having a first side and a second side;
a flexible side wall coupled to the base to form an enclosure adjacent the first side;
a biasing member disposed in the enclosure and configured to bias the blister to the expanded position;
a first check valve coupled to the base and fluidly coupled to the absorbent and the enclosure, the first check valve configured to prevent fluid flow from the blister into the absorbent if the blister is moved from the expanded position to the collapsed position; and
a second check valve fluidly coupled to the blister and an ambient environment, the second check valve configured to prevent fluid flow from the ambient environment into the blister if the blister is moved from the collapsed position to the expanded position.

2. The system of claim 1, wherein the base further comprises:
an inlet recess disposed in the first side of the base;
an inlet channel disposed in the second side of the base, the inlet channel fluidly coupled to the inlet recess and a periphery of the base;
an exhaust recess disposed in the first side of the base, the exhaust recess proximate to the inlet recess;
an exhaust channel disposed in the second side of the base, the exhaust channel fluidly coupled to the exhaust recess and a periphery of the base;
the first check valve is disposed in the inlet recess; and
the second check valve is disposed in the exhaust recess.

3. The system of claim 1, wherein the blister further comprises:
a top coupled to the flexible side wall opposite the base, the top having a first side and a second side;
an exhaust recess disposed in the second side of the top;
wherein the base further comprises:
an inlet recess disposed in the first side of the base;
an inlet channel disposed in the second side of the base, the inlet channel fluidly coupled to the inlet recess and a periphery of the base;
the first check valve is disposed in the inlet recess; and
the second check valve is disposed in the exhaust recess.

4. The system of claim 3, wherein the top is formed from an elastomeric material.

5. The system of claim 1, wherein the base is formed from an elastomeric material.

6. The system of claim 1, wherein the first check valve is an umbrella valve.

7. The system of claim 1, wherein the second check valve is an umbrella valve.

8. The system of claim 1, wherein the biasing member is a foam.

9. The system of claim 8, wherein the foam comprises a cylinder having a first end, a second end, a side wall, and a plurality of holes extending from the first end to the second end.

10. The system of claim 9, wherein each of the plurality of holes has a diameter of about 3 mm.

11. The system of claim 9, wherein each of the plurality of holes has a diameter of about 5 mm.

12. A method of manufacturing a dressing assembly for negative-pressure therapy, the method comprising:
providing a pouch;
disposing a cover over the pouch and coupling the pouch to the cover;
providing a disc having a first side and a second side;
coupling the second side of the disc to the cover;
positioning a foam block adjacent the first side of the disc;
coupling a film to the disc to form a source cavity enclosing the foam block adjacent the first side and fluidly coupling the source cavity to the pouch through the disc;
coupling a first check valve to the disc and fluidly coupling the first check valve to the pouch and the source cavity, the first check valve configured to prevent fluid flow from the source cavity into the pouch; and
fluidly coupling a second check valve to the source cavity and an ambient environment, the second check valve configured to prevent fluid flow from the ambient environment into the source cavity.

13. The method of claim 12, wherein the method further comprises:
forming an inlet recess in the first side of the disc;
forming an inlet channel in the second side of the disc, the inlet channel fluidly coupled to the inlet recess and a periphery of the disc;
forming an exhaust recess in the first side of the disc, the exhaust recess proximate to the inlet recess;
forming an exhaust channel in the second side of the disc, the exhaust channel fluidly coupled to the exhaust recess and a periphery of the disc;
disposing the first check valve in the inlet recess; and
disposing the second check valve in the exhaust recess.

14. The method of claim 12, wherein the method further comprises:
providing a top having a first side and a second side;
forming an exhaust recess in the second side of the top;
coupling the second side of the top to the film opposite the disc;
forming an inlet recess in the first side of the disc;
forming an inlet channel in the second side of the disc, the inlet channel fluidly coupled to the inlet recess and a periphery of the disc;
disposing the first check valve in the inlet recess; and
disposing the second check valve in the exhaust recess.

15. A dressing assembly for providing negative-pressure therapy to a tissue site, the dressing assembly comprising:
a pouch;
a cover configured to cover the pouch;
a negative-pressure source fluidly coupled to the pouch and having a first position and a second position, the negative-pressure source comprising:
a disc having a first side and a second side;
a film coupled to the disc to form a source cavity adjacent the first side;
a foam block disposed in the source cavity and configured to bias the negative-pressure source to the second position;
a first check valve coupled to the disc and fluidly coupled to the pouch and the source cavity, the first check valve configured to prevent fluid flow from the negative-pressure source into the pouch if the negative-pressure source is moved from the second position to the first position; and
a second check valve fluidly coupled to the negative-pressure source and an ambient environment, the second check valve configured to prevent fluid flow from the ambient environment into the negative-pressure source if the negative-pressure source is moved from the first position to the second position.

16. The dressing assembly of claim 15, wherein the disc further comprises:
an inlet recess disposed in the first side of the disc;
an inlet channel disposed in the second side of the disc, the inlet channel fluidly coupled to the inlet recess and a periphery of the disc;
an exhaust recess disposed in the first side of the disc, the exhaust recess proximate to the inlet recess;
an exhaust channel disposed in the second side of the disc, the exhaust channel fluidly coupled to the exhaust recess and a periphery of the disc;
wherein the first check valve is disposed in the inlet recess; and
wherein the second check valve is disposed in the exhaust recess.

17. The dressing assembly of claim 15, wherein the negative-pressure source further comprises:
a top coupled to the film opposite the disc, the top having a first side and a second side;
an exhaust recess disposed in the second side of the top;
wherein the disc further comprises:
an inlet recess disposed in the first side of the disc;
an inlet channel disposed in the second side of the disc, the inlet channel fluidly coupled to the inlet recess and a periphery of the disc;
wherein the first check valve is disposed in the inlet recess; and
wherein the second check valve is disposed in the exhaust recess.

18. The dressing assembly of claim 15, wherein:
the pouch is coupled to the cover, the cover having a periphery extending beyond an edge of the pouch; and
the negative-pressure source is coupled to the cover.

19. The dressing assembly of claim 18, wherein the periphery of the cover further comprises:
a barrier layer;
a bonding adhesive layer coupled to the barrier layer; and
a sealing adhesive layer having a plurality of apertures and coupled to the barrier layer, the bonding adhesive layer is configured to extend at least partially through the plurality of apertures in the sealing adhesive layer in response to force applied to the barrier layer.

20. The dressing assembly of claim 15, wherein the foam block comprises:
a first foam block having a first compressive force deflection; and
a second foam block having a second compressive force deflection.

21. The dressing assembly of claim 15, wherein the pouch further comprises:
an upstream layer;
an absorbent disposed adjacent to the upstream layer; and
a downstream layer disposed adjacent to the absorbent, the upstream layer and the downstream layer having peripheral portions coupled to each other to enclose the absorbent.

22. (canceled)
